(19) United States
(12) Patent Application Publication
(10) Pub. No.: US 2022/0056517 A1
(43) Pub. Date: Feb. 24, 2022

(54) PORE

(71) Applicants: Oxford Nanopore Technologies Limited, Oxford (GB); VIB VZW, Gent (BE); Vrije Universiteit Brussel, Brussels (BE)

(72) Inventors: Han Remaut, Roosbeek (BE); Sander Van Der Verren, Oxford (GB); Nani Van Gerven, Oxford (GB); Lakmal Nishantha Jayasinghe, Oxford (GB); Elizabeth Jayne Wallace, Oxford (GB); Pratik Raj Singh, Oxford (GB); Richard George Hambley, Oxford (GB); Michael Robert Jordan, Oxford (GB); John Joseph Kilgour, Oxford (GB); Andrew John Heron, Oxford (GB)

(73) Assignees: Oxford Nanopore Technologies Limited, Oxford (GB); VIB VZW, Gent (GB); Vrije Universiteit Brussel, Brussels (GB)

(21) Appl. No.: 17/291,656

(22) PCT Filed: Nov. 7, 2019
(86) PCT No.: PCT/GB2019/053153

§ 371 Date: May 6, 2021

(30) Foreign Application Priority Data
Nov. 8, 2018 (GB) 1818216.2
Nov. 22, 2018 (GB) 1819054.6

(51) Int. Cl. C12Q 1/6869

(52) U.S. Cl. CPC C12Q 1/6869

(57) ABSTRACT

A system for characterising a target polynucleotide, the system comprising a membrane and a pore complex; wherein the pore complex comprises: (i) a nanopore located in the membrane, and (ii) an auxiliary protein or peptide attached to the nanopore; wherein the nanopore and the auxiliary protein or peptide together form a continuous channel across the membrane, the channel comprising a first constriction region and a second constriction region; wherein the first constriction region is formed by a portion of the nanopore, and wherein the second constriction region is formed by at least a portion of the auxiliary protein or peptide.

### RELATED APPLICATIONS

This application is a national stage filing under 35 U.S.C. § 371 of international application number PCT/GB2019/053153, filed Nov. 7, 2019, which claims the benefit of United Kingdom application serial numbers 1818216.2, filed Nov. 8, 2018, and 1819054.6, filed Nov. 22, 2018, each of which is herein incorporated by reference in its entirety.

### FIELD

The present invention relates to novel nanopore complexes, systems comprising a membrane and the novel nanopore complexes for characterising polynucleotides, and methods of characterising polynucleotides using the systems.

### BACKGROUND

Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore. Analytes can be organic and inorganic small molecules as well as various biological or synthetic macromolecules and polymers including polynucleotides, polypeptides and polysaccharides. Nanopore sensing can reveal the identity and perform single molecule counting of the sensed analytes, but can also provide information on the analyte composition such as nucleotide, amino acid or glycan sequence, as well as the presence of base, amino acid or glycan modifications such as methylation and acylation, phosphorylation, hydroxylation, oxidation, reduction, glycosylation, decarboxylation, deamination and more. Nanopore sensing has the potential to allow rapid and cheap polynucleotide sequencing, providing single molecule sequence reads of polynucleotides of tens to tens of thousands bases length.

Two of the essential components of polymer characterization using nanopore sensing are (1) the control of polymer movement through the pore and (2) the discrimination of the composing building blocks as the polymer is moved through the pore. During nanopore sensing, the narrowest part of the pore forms the reader head, the most discriminating part of the nanopore with respect to the current signatures as a function of the passing analyte.

For analytes being polynucleotides, nucleotide discrimination is achieved via passage through such a mutant pore, but current signatures have been shown to be sequence dependent, and multiple nucleotides contributed to the observed current, so that the height of the channel constriction and extent of the interaction surface with the analyte affect the relationship between observed current and polynucleotide sequence. While the current range for nucleotide discrimination has been improved through mutation of the CsgG pore, a sequencing system would have higher performance if the current differences between nucleotides could be improved further. Accordingly, there is a need to identify novel ways to improve nanopore sensing features.

### SUMMARY

The disclosure relates to a system for characterising a target polynucleotide. The system comprises a membrane in which a transmembrane pore in present. The pore is a complex of a transmembrane nanopore and an auxiliary protein, or auxiliary peptide. The pore comprises at least two constrictions, which can function as reader heads in polynucleotide characterisation methods, wherein a first constriction is present in the transmembrane nanopore and a second constriction is provided by the auxiliary protein or auxiliary peptide. As the pore has at least two constrictions, which can function as sites capable of discriminating between different nucleotides, the pore displays improved nucleotide recognition. The pore is therefore advantageous for sequencing polynucleotides. The presence in a pore of more than one site that is capable of discriminating between different nucleotides not only allows the length of a nucleic acid sequence to be determined, but also allows the sequence of a polynucleotide to be determined more efficiently.

In particular, the multiple reader head pore complex described herein may provide improved base calling, i.e. sequencing, of homopolymeric stretches of nucleotides. A sharp constriction may serve as a reader head of a pore and be able to discriminate a mixed sequence of A,C,G and T as it passes through the pore. This is because the measured signal contains characteristic current deflections generated as each nucleotide interacts with the constriction, from which the identity of the sequence can be derived. However, in homopolymeric regions of DNA, the measured signal may not show current deflections of sufficient magnitude to allow single base identification; such that an accurate determination of the length of a homopolymer cannot be made from the magnitude of the measured signal alone. Introducing a second constriction using an auxiliary protein or peptide in conjunction with a transmembrane nanopore that interacts with nucleotides spatially separated from the nucleotides that are interacting with the first constriction, results in signal steps being produced that contain information allowing a homopolymeric sequence to be determined more accurately, particularly for longer stretches of homopolymeric sequences, than when the transmembrane pore is used without the auxiliary protein or peptide.

In a first aspect, the invention provides a system for characterising a target polynucleotide, the system comprising a membrane and a pore complex, wherein the pore complex comprises: (i) a nanopore located in the membrane, and (ii) an auxiliary protein or peptide attached to the nanopore, wherein the nanopore and the auxiliary protein or peptide together form a continuous channel across the membrane, the channel comprising a first constriction region formed by a portion of the nanopore and a second constriction region formed by at least a portion of the auxiliary protein or peptide.

In one embodiment, the auxiliary protein is a multimeric protein.

In one embodiment, the auxiliary protein is a transmembrane protein nanopore or a fragment thereof. In certain embodiments, the transmembrane protein nanopore is selected from MspA, α-hemolysin, CsgG, lysenin, InvG, GspD, leukocidin, FraC, aerolysin, NetB, and functional homologues and fragments thereof.

In one embodiment, the auxiliary protein comprises a fragment of a component of a transmembrane protein pore complex.

In one embodiment the auxiliary protein is one that does not naturally form a nanopore in a membrane and/or does not comprise a component, or a fragment thereof, of a transmembrane pore complex that forms naturally in a membrane.

In one embodiment, the auxiliary protein or peptide is ring-shaped. In one embodiment, the auxiliary protein or peptide is a ring-shaped protein or peptide that does not naturally form a nanopore in a membrane and/or does not comprise a component, or a fragment thereof, of a transmembrane pore complex that forms naturally in a membrane. In certain embodiments, the auxiliary protein is selected from GroES, CsgF or a CsgF peptide, pentraxin, SP1, and functional homologues and fragments thereof.

In some embodiments, the auxiliary protein is a transmembrane protein nanopore or a fragment thereof. For example, in certain embodiments, the transmembrane protein pore is selected from MspA, α-hemolysin, CsgG, lysenin, InvG, GspD, leukocidin, FraC, aerolysin, NetB, and functional homologues and fragments thereof. In a particular embodiment, when the nanopore is a CsgG pore, the auxiliary protein is not CsgF, or a homologue, fragment or modified version thereof.

In one embodiment, the nanopore in the complex is a first transmembrane protein nanopore and the auxiliary protein is a second transmembrane protein nanopore, or a fragment thereof. In some embodiments, the first transmembrane protein nanopore and the second transmembrane protein nanopore, or fragment thereof, are of the same transmembrane protein nanopore type. In some more particular embodiments, the first transmembrane protein nanopore and the second transmembrane protein nanopore are the same. In other embodiments, the first transmembrane protein nanopore and the second transmembrane protein nanopore, or fragment thereof, are of different transmembrane protein nanopore types. In a particular embodiment, when the first transmembrane protein nanopore is a CsgG pore, or a homologue, fragment or modified version thereof, the second transmembrane protein nanopore is not a CsgG nanopore, or a homologue, fragment or modified version thereof. Conversely, when the second transmembrane protein nanopore is a CsgG nanopore, or a homologue, fragment or modified version thereof, the first transmembrane protein nanopore is not a CsgG nanopore, or a homologue, fragment or modified version thereof.

In some embodiments, the first transmembrane protein nanopore and/or the second transmembrane protein nanopore, or fragment thereof, are homooligomers. In other embodiments, the first transmembrane protein nanopore and/or the second transmembrane protein nanopore, or fragment thereof, are heterooligomers.

In one embodiment, the nanopore is selected from MspA, CsgG, and functional homologues and fragments thereof, and wherein the auxiliary protein is GroES or a functional homologue or fragment thereof.

In some embodiments, the first and/or second transmembrane protein nanopore comprises at least one amino acid modification compared to the corresponding naturally occurring transmembrane protein nanopore. The modified transmembrane protein nanopore may, for example, comprise: (i) at least one amino acid residue at the interface between the transmembrane protein nanopore and the auxiliary protein, which amino acid residue is not present in the corresponding naturally occurring transmembrane protein nanopore; and/or (ii) at least one amino acid residue that forms part of the first constriction, which amino acid residue is not present in the corresponding naturally occurring transmembrane protein nanopore.

In one embodiment, the membrane comprises a layer of amphipathic molecules and/or the membrane is or comprises a solid state layer. In one embodiment, the nanopore is a solid state nanopore formed in the solid state layer.

In the pore complex, in one embodiment, at least a portion of the auxiliary protein or peptide is located within the lumen of the nanopore. The second constriction may, for example, be formed by at least a portion of the auxiliary protein or peptide, which portion is located within the lumen of the nanopore. In one embodiment, the auxiliary protein or peptide is located entirely within the lumen of the nanopore. In another embodiment, the auxiliary protein or peptide is located outside the lumen of the nanopore.

In one embodiment, the auxiliary protein or peptide is attached to the nanopore via one or more covalent bonds and/or via one or more non-covalent interactions.

In some embodiments, the auxiliary protein is a modified auxiliary protein or peptide comprising at least one amino acid modification compared to the corresponding naturally occurring auxiliary protein or peptide. For example, the modified auxiliary protein or peptide comprises: (i) at least one amino acid residue at the interface between the transmembrane protein nanopore and the auxiliary protein or peptide, which amino acid residue is not present in the corresponding naturally occurring auxiliary protein or peptide; and/or (ii) at least one amino acid residue that forms part of the second constriction, which amino acid residue is not present in the corresponding naturally occurring auxiliary protein or peptide.

In the pore complex of one embodiment, the first constriction and/or the second constriction has a minimum diameter of from about 0.5 nm to about 2 nm, or about 0.5 nm to about 4 nm.

In a further embodiment, the system is suitable for characterising a target polynucleotide comprising a homopolymeric region.

In some embodiments, the system further comprises a first chamber and a second chamber, wherein the first and second chambers are separated by the membrane. In one embodiment, a target polynucleotide is transiently located within the continuous channel and wherein one end of the target polynucleotide is located in the first chamber and one end of the target polynucleotide is located in the second chamber. The system may still further comprise an electrically-conductive solution in contact with the nanopore, electrodes providing a voltage potential across the membrane, and a measurement system for measuring the current through the nanopore.

In a second aspect, the disclosure relates to an isolated pore complex comprising (i) a nanopore, and (ii) an auxiliary protein or peptide attached to the nanopore;

wherein the nanopore and the auxiliary protein or peptide together define a continuous channel, the channel comprising a first constriction region and a second constriction region;

wherein the first constriction region is formed by a portion of the nanopore, and wherein the second constriction region is formed by at least a portion of the auxiliary protein or peptide.

The isolated pore complex may have any one or more of the features described herein with reference to the first aspect of the invention.

In a third aspect, the disclosure relates to a method for characterising a target polynucleotide, the method comprising the steps of:

(a) contacting a system as disclosed herein with the target polynucleotide;

(b) applying a potential across the membrane such that the target polynucleotide enters the continuous channel formed by the pore complex; and

(c) taking one or more measurements as the polynucleotide moves with respect to the continuous channel, thereby characterising the polynucleotide.

In one embodiment, step (c) comprises measuring the current passing through the continuous channel, wherein the current is indicative of the presence and/or one or more characteristics of the target polynucleotide and thereby detecting and/or characterising the target polynucleotide. In an embodiment of the method, the nucleotides in the target polynucleotide interact with the first and second constriction regions within the continuous channel and wherein each of the first and second constriction regions is capable of discriminating between different nucleotides, such that the overall current passing through the continuous channel is influenced by the interactions between each of the first and second constriction regions and the nucleotides located at each of the regions. In one embodiment, the polynucleotide moves through the channel and translocates across the membrane. In one embodiment, a polynucleotide binding protein is used to control the movement of the polynucleotide with respect to the pore. In one embodiment, the characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified. In one embodiment, the method comprises determining the nucleotide sequence of the target polynucleotide. The target polynucleotide, in one embodiment, comprises a homopolymeric region.

### BRIEF DESCRIPTION OF THE FIGURES

FIG. 1 shows the structure of a pore complex comprising a CsgG pore as a transmembrane nanopore and a second CsgG pore as an auxiliary protein. The two CsgG pores are in a tail to tail orientation and the two reader heads are indicated.

FIG. 2 shows holes in the walls of the CsgG pore complex (double pore) shown in FIG. 1. The inventors have produced data suggesting that double pore current is less than half the single pore current (at higher voltages). The inventors have proposed that this could be due to current leak from side pockets at the interface of the two pores. These gaps can be filled in by changing one or more amino acid residues in this area to bulkier amino acid residues.

FIG. 3 shows the structure of part of the interface between two CsgG pores in the CsgG pore complex (double pore) shown in FIG. 1. The mutations are shown in a pore that comprises Y51A and F56Q mutations (AQ=CP1-(WT-Y51A/F56Q-StrepII(C))9). The indicated Cys mutant pairs may form S—S bonds.

FIG. 4 shows (Left) the structure of part of a CsgG pore complex (double pore) as shown in FIG. 1 with a single stranded DNA molecule inserted in the pore. There are approximately 15 nucleotides between the two constrictions (reader heads). The two reader-heads are separated by a non-DNA interacting region. Also shown based on modelling data are (Middle) a visualization of the channel through the pore complex, and (Right) a pore radius profile showing the pore radius of the channel through the pore complex.

FIG. 5A shows the cross section of a CsgG pore showing the constriction (reader head) with a single stranded DNA inserted.

FIG. 5B shows the cross section of a wild type CsgG pore in which the three main amino acid residues, F56 (side chain residues at top of central ring, mid-grey), N55 (central ring, dark grey) and Y51 (bottom of central ring, light grey), are indicated. The constriction is located within the barrel (at the top) in a relatively unstructured loop. The reader head can be elongated either by mutations at existing positions or by inserting additional amino acid residues. For example, the reader head can be broadened by mutations at each of the three indicated positions and/or by mutations at the 52, 53 and 54 positions.

FIG. 5C shows the positions of the residues from K49 to F56 in a monomer of the CsgG pore. 51 can be moved further down by increasing the length of the loop in between 51 and 55. New amino acid residues can be inserted between 51 and 52, 52 and 53, 53 and 54 or 54 and 55. For example, 1, 2, 3 or more amino acid residues may be inserted. To keep the flexible nature of the loop, A/S/G/T can be inserted. To add a kink to the loop P can be inserted. New A amino acid residues could contribute to the signal (e.g. S/T/N/Q/M/F/W/Y/V/I). Similarly, new amino acids can be inserted between 55 and 56 (1 or 2 or more). They can be any of the above amino acids. Y51 can also move downwards by inserting amino acids to both sides of the loop above Y51. For example S or G or SG or SGG or SGS or GS or GSS or GSG or other suitable amino acid (1 or 2 or more) can be inserted (i) between (49 and 50) and between (52 and 53); (ii) between (50 and 51) and between (51 and 52); (iii) combinations of 1 and 2; or (iv) any of (i) to (iii) can be combined with other insertions (e.g. insertions between 55 and 56).

FIG. 6 shows the structures and reader heads of the baseline CsgG pore used in the Examples (A), a CsgG pore with an elongated reader head (B) and a double CsgG pore (C). Homopolymer basecalling is improved compared to the baseline when the elongated reader head pore or the double pore is used.

FIG. 7 shows the structure of CsgG pore and the interface for complex formation with CsgF. Cross-sectional (A), side (B) and top (C) views of CsgG oligomers (e.g., nonamers) in surface (A) and ribbon (B, C) representation, with a single CsgG protomer coloured light grey (D) (based on the CsgG X-ray structure PDB entry: 4uv3). The CsgG constriction loop (CL loop) spans residues 46 to 61 according SEQ ID NO:3, and is indicated in dark grey in all panels, and corresponds to the loop provided in the bottom left of (E). CsgG residues for which the side chain faces the inner lumen of the CsgG beta-barrel are coloured mid-grey as indicated and labelled in the 0 strands in (E) and (D). These residues represent sites that can be used for substitution to natural or non-natural amino acids, e.g., amenable for attachment (e.g., covalent crosslinking) of a pore-resident peptide, (including e.g., a modified CsgF peptide, or a homologue thereof) to a CsgG pore or monomer. In some embodiments crosslinking residues include Cys and reactive and photo-reactive amino acids, acids such as azidohomoalanine, homopropargylglycyine, homoallelglycine, p-acetyl-Phe, p-azido-Phe, p-propargyloxy-Phe and p-benzoyl-Phe (Wang et al. 2012; Chin et al. 2002) and can be substituted into positions 132, 133, 136, 138, 140, 142, 144, 145, 147, 149, 151, 153, 155, 183, 185, 187, 189, 191, 201, 203, 205, 207 or 209 according to SEQ ID NO:3. (E) shows a zoom of the CL loop and the transmembrane beta-strands of a CsgG monomer. The CsgG constriction loop (coloured dark blue) forms the orifice or narrowest passage in the CsgG pore (panel A). In some embodiments, three positions in the CL loop, 56, 55 and 51 according to SED ID NO:3, are of particular importance to the diameter and chemical and physical properties of the CsgG channel orifice or “reader head”. These represent preferred positions to alter the nanopore sensing properties of CsgG pores and homologues.

FIG. 8 shows the CsgG:CsgF structure as determined in cryo-EM. (A) A cryo electron micrograph of the CsgG:CsgF complex shows the presence of 9-mer and 18-mer CsgG:CsgF complexes, with a number of single particles of the 9- and 18-mer forms highlighted by full and dashed circles, respectively. (B) Two representative class averages of the CsgG:CsgF 9-mer complex, viewed from the side. Class averages include 6020 and 4159 individual particles, respectively. The class averages reveal the presence of additional density on top of the CsgG particle, corresponding to an oligomeric complex of CsgF. Three distinct regions can be seen in the CsgF oligomer: a “head” and “neck” region, as well as a region that resides inside lumen of the CsgG beta-barrel and forms a constriction or narrow passage (labelled F) that is stacked on top of the constriction formed by the CsgG CL loop (labelled G). This latter CsgF region is referred to as CsgF Constriction Peptide (FCP).

FIG. 9 shows the three-dimensional structural model of a CsgG:CsgF complex. Cross-sectional views of the 3D cryoEM electron density of the CsgG:CsgF 9-mer complex calculated from 20.000 particles assigned to 21 class averages. The right picture shows a superimposition with the CsgG 9-mer X-ray structure (PDB entry: 4uv3) docked into the cryoEM density. The regions corresponding to CsgG, CsgF and the CsgF head, neck and FCP domains are indicated. The cross-sections show the CsgF FCP regions forms an additional constriction (labelled F) in the CsgG channel, approximately 2 nm above the CsgG constriction loop (labelled G).

FIG. 10 shows the experimental evaluation of the E. coli CsgF region forming the CsgG-interaction sequence and CsgF constriction peptide (FCP). Panel (A) shows the mature sequences (i.e. after removal of the CsgF signal peptide, corresponding to residues 1-19 of SEQ ID NO:5) of the four N-terminal CsgF fragments (SEQ ID NO:8_CsgF residues 1-27; SEQ ID NO: 10; SEQ ID NO: 12 and SEQ ID NO: 14) that were co-expressed with E. coli CsgG (SEQ

ID NO:2). (B) Anti-Strep (left) and anti-His (right) Western blot analysis of SDS-PAGE runs of crude cell lysates of CsgG and CsgF co-expression experiments. Anti-strep analysis demonstrates the expression of CsgG in all co-expression experiments, whereas anti-his western blot analysis shows detectible levels of CsgF fragments only for the truncation mutant CsgF 1-64 (SEQ ID NO: 14). A His-tagged nanobody (Nb) was used as positive control. (C) Anti-His dot blot analysis of the presence of CsgF fragments in CsgG:CsgF co-expression experiments. Top row shows whole cell lysates, middle and bottom rows show the eluate and flowthrough of a Strep affinity pulldown experiment. These data demonstrate that CsgF fragment 1-64, and to a much lesser extent CsgF 1-48, is specifically pulled down as a complex with Strep-tagged CsgG. CsgF fragments 1-27 and 1-38 do not result in detectable levels of the corresponding CsgF fragments and show no sign of complex formation with CsgG.

FIG. 11 shows the high resolution cryoEM structure of the CsgG:CsgF complex. CsgG is shown in light grey and CsgF is shown in dark grey. A. Final electron density map of the CsgG:CsgF complex at 3.4 Å resolution. Side view. B. Top view of the cryoEM structure to show CsgG:CsgF comprises a 9:9 stoichiometry, with C9 symmetry. C. Internal architecture of the CsgG:CsgF complex. GC, CsgG constriction, FC, CsgF constriction. D. Interactions between CsgG and CsgF proteins. CsgG and the CsgG constriction are coloured light grey and grey respectively. CsgF is coloured dark grey. Residues in CsgG and CsgF are labelled in light grey and black respectively.

FIG. 12 shows the two reader heads of the CsgG:CsgF complex. CsgG is shown in light grey and reader head of the CsgG pore is shown in dark grey. CsgF is shown in black and the reader head of the CsgF is labelled.

FIG. 13 shows the heat stability of CsgG:CsgF complexes. M: Molecular weight marker, Lane 1: CsgG pore, Lane 2: CsgG:CsgF complex at room temperature: Lanes 3-9: CsgG:CsgF sample was heated at different temperatures (40, 50, 60, 70, 80, 90,100° C. respectively) for 10 minutes. Lane 1: A. Y51A/F56Q/N55V/N91R/K94Q/R97W-del(V105-I107):CsgF-(1-45). B. Y51A/F56Q/N55V/N91R/K94Q/R97W-del(V105-I107):CsgF-(1-35). C. Y51A/F56Q/N55V/N91R/K94Q/R97W-del(V105-107):CsgF-(1-30). Samples were subjected to SDS-PAGE on a 7.5% TGX gel. CsgG:CsgF complexes with both CsgF-(1-45) and CsgF-(1-35) shows a shift from the CsgG pore band in lanes 1. Therefore, it is clear that both those complexes are heat stable up to 90° C. The complex and the pore breaks down to CsgG monomers at 100 C (lanes 9). Although the same heat stability pattern is seen with the CsgG:CsgF complex with CsgF-(1-30), its difficult to see the shift between the protein bands of the CsgG pore(lane 1) and CsgG-CsgF complexes (lanes 2-8).

FIG. 14 shows CsgG:CsgF formation via in vitro reconstitution using synthetic CsgF peptides. Native PAGE showing CsgG:CsgF formation via in vitro reconstitution using wildtype CsgG or a CsgG mutant with altered constriction Y51A/F56Q/K94Q/R97W/R192D-del(V105-I107). An Alexa 594-labelled CsgF peptide corresponding to the first 34 residues of mature CsgF (Seq ID No 6) was added to purified Strep-tagged CsgG or Y51A/F56Q/K94Q/R97W/R192D-del(V105-I107) in 50 mM Tris, 100 mM NaCl, 1 mM EDTA, 5 mM LDAO/C8D4 in a 2:1 molar ratio during 15 minutes at room temperature to allow reconstitution. After pull down of CsgG-strep on StrepTactin beads, the sample was analysed on native-PAGE. Both WT and Y51A/F56Q/K94Q/R97W/R192D-del(V105-I107) CsgG bind the CsgF N-terminal peptide as visualised by the fluorescence tag.

FIG. 15 shows stabilising CsgG:CsgF or CsgG:FCP complexes. A. Identified amino acid positions of CsgG (SEQ ID NO: 3 and CsgF (SEQ ID NO:. 6) pairs where S—S bonds can be made. B. Schematic representation to show the S—S bond between CsgG-Q153C and CsgF-G1C.

FIG. 16 shows cysteine cross linking of the CsgG:CsgF complex. A. Y51A/F56Q/N91R/K94Q/R97W/Q153C-del(V105-I107) and CsgF-G1C proteins were purified separately and incubated together at 4° C. for lhour or overnight to form the complex and allow S—S formation. No oxidising agents were added to promote S—S formation. Control CsgG pore (Y51A/F56Q/N91R/K94Q/R97W/Q153C-DEL(V105-I107)) and complex (with and without

DTT) were heated at 100° C. for 10 minutes to breakdown the complex into CsgG monomer (CsgGm, 30 KDa) and CsgF monomer (CsgFm, 15 KDa). A dimer between the CsgGm and CsgFm (CsgGm-CsgFm, 45 KDa) can be seen in the absence of the reducing agents confirming the S—S bond formation. Increased dimer formation can be seen in overnight incubation compared to one hour incubation. B. Mass spectrometry analysis was carried out on the gel purified CsgGm-CsgFm band from overnight incubation. Protein was proteolytically cleaved to generate tryptic peptides. LC-MS/MS sequencing methods were performed, resulting in the identification of the precursor ion above, corresponding to the linked peptides shown. This precursor ion was fragmented to give the fragment ions observed. These include ions for each of the peptides, as well as fragments incorporating the intact disulphide bond. This data provides strong evidence for the presence of a disulphide bond between C1 of CsgF and C153 of CsgG.

FIG. 17 shows the improved efficiency of Cysteine cross linking of the CsgG:CsgF complex. Lane 1: Y51A/F56Q/N91R/K94Q/R97W/N133C-del(V105-I107)and CsgF-T4C proteins were co expressed the CsgG:CsgF complex was purified. Lane 2: The complex was heated in the presence of DTT to break down the complex into substituent monomers (CsgGm and CsgFm). DTT will break down any S—S bonds between CsgG-N133C and CsgF-T4C if formed. Lane 3: The complex is incubated with the oxidising agent copper-orthophenanthroline to promote S—S bond formation. Lane 4: Oxidised sample was heated at 100° C. in the absence of DTT to break down the complex. A new band of 45 KDa corresponding to the CsgGm-CsgFm appears confirming the S—S bond formation.

FIG. 18 shows the current signature when the DNA strand is passing through the CsgG:CsgF complex. The complexes were made by co-expressing the CsgG pore (Y51A/F56Q/N91R/K94Q/R97W-del(V105-I107)) containing the C terminal strep tag with the full length CsgF proteins containing C terminal His tag and TEV protease cleavage site between 35 and 36 of seq ID no. 6. Purified complexes were then cleaved by TEV protease to make the given CsgG:CsgF complexes. Note that TEV cleavage leaves ENLYFQ sequence at the cleavage site. A. No mutations at 17 position of CsgF. B. N175 mutation in CsgF.

FIG. 19 shows the current signature when the DNA strand is passing through the CsgG:CsgF complex. The complexes were made by incubating Y51A/N55V/F56Q/N91R/K94Q/R97W-del(V105-I107)pore containing the C terminal strep tag with CsgF-(1-35) mutants. A. CsgF-N175-(1-35). B. CsgF-N17V-(1-35).

FIG. 20 shows the current signature when the DNA strand is passing through the CsgG:CsgF complex. The complexes were made by incubating different CsgG pores containing the C terminal strep tag with CsgF-N175-(1-35). A. CsgG pore is Y51A/N55V/F56Q/N91R/K94Q/R97W-del(V105-I107). B. CsgG pore is Y51T/N55V/F56Q/N91R/K94Q/R97W-del(V105-I107). C. CsgG pore is Y51A/N551/F56Q/N91R/K94Q/R97W-del(V105-I107). D. CsgG pore is Y51A/F56A/N91R/K94Q/R97W-del(V105-I107). E. CsgG pore is Y51A/F561/N91R/K94Q/R97W-del(V105-I107). F. CsgG pore is Y515/N55V/F56Q/N91R/K94Q/R97W-del(V105-I107).

FIG. 21 shows the current signature when the DNA strand is passing through the CsgG:CsgF complex. Complexes were made by incubating the E. coli purified Y51A/N55V/F56Q/N91R/K94Q/R97W-del(V105-I107) pore containing the C terminal strep with CsgF of three different lengths. A. CsgF-(1-29), B. CsgF-(1-35), C. CsgF-(1-45). The arrow indicates the range of the signal. Surprisingly, complex with the CsgF-(1-29) produces the signal with the largest range.

FIG. 22 shows the signal:noise of the current signature when the DNA strand is passing through the CsgG:CsgF complex. Different CsgG:CsgF complexes were made by incubating different CsgG pores (1-Y51A/F56Q/N91R/K94Q/R97W-del(V105-I107) 2-Y51A/N55I/F56Q/N91R/K94Q/R97W-del(V105-I107) 3-Y51A/N55V/ F56Q/N91R/K94Q/R97W-del(V105-I107) 4-Y51A/F56A/N91R/K94Q/R97W-del(V105-I107) 5-Y51A/F561/N91R/K94Q/R97W-del(V105-I107) 6-Y51A/F56V/N91R/K94Q/R97W-del(V105-I107) 7-Y51S/N55A/F56Q/N91R/K94Q/R97W-del(V105-I107) 8-Y51S/N55V/ F56Q/N91R/K94Q/R97W-del(V105-I107) 9-Y51T/N55V/ F56Q/N91R/K94Q/R97W-del(V105-I107)) with the same CsgF peptide CsgF-(1-35). Different squiggle patterns were observed in DNA translocation experiments and their signal:noise is measured. Higher accuracies can be obtained with larger signal:noise ratios.

FIG. 23 shows the sequencing errors with narrow reader-heads. A representation of DNA base interaction with the reader head of the CsgG pore. Approximately, 5 bases dominate the current signal at any given time when the DNA strand is translocating through the pore. B. Mapping plots of the signal. Event-detected signal for multiple reads mapped to modelled signal using a custom HMM, for a mixed sequence lacking homopolymer runs, and for a sequence containing three homopolymer runs of 10 T.

FIG. 24 shows mapping of the reader heads of the CsgG:CsgF complex. Reader head discrimination plot for the CsgG:CsgF complex. The average variation in modelled current when the base at each read head position is varied. To calculate the read head discrimination at position i for a model of length k with alphabet of length n, we define the discrimination at read-head position i as the median of the standard deviations in current level for each of the nk-1 groups of size n where position i is varied while other positions are held constant. B. Static DNA strands to map the reader head: A set of polyA DNA strands (SS20 to SS38) in which one base is missing from the DNA backbone (iSpc3) is created. In each strand, the position of iSpc3 moves from 3′ end towards the 5′ end. Based on previous experiments with the CsgG pore, 7th position of the DNA is expected to be located within the CsgG constriction. SS26 corresponds to this DNA is highlighted. Based on the model from (A), 4-5 bases are expected to separate CsgG and CsgF reader heads. Therefore, approximately, position 12 and 13 are expected to be within the CsgF constriction. SS31 and SS32 DNA strands corresponding to those positions are highlighted. C and D. Mapping the two reader heads: Biotin modification at the 3′ end of each strand is complexed with monovalent streptavidin and the current blockage generated from each strand is recorded in a MinION set up. When the iSpc3 position is present above or below the constriction within the pore, no deflection is expected. However, when the iSpc3 is located within the constriction, a higher current level is expected to pass through the pore—the extra space created by the lack of base lets more ions to pass through. Therefore, by plotting the current passing through with each DNA strand, the locations of the two reader heads can be mapped. As expected, the highest deflection in the current is seen when the position 7 of the DNA strand is occupied by iSpc3 (C). iSpc3 at positions 6 and 8 also produce a higher deflection over the average polyA current level. Therefore, positions 6, 7 and 8 of the DNA strand represent the first reader head—CsgG reader head. As expected, when positions 12th and 13th are occupied by iCsp3, another deviation from baseline polyA is observed (D). This indicates the second reader head of the pore—CsgF reader head. Results also confirm that the two reader heads are apart by approximately 4-5 bases.

FIG. 25 shows the reader head discrimination and base contribution. Left hand panel demonstrates the read-head discrimination of each mutant pore: the average variation in modelled current when the base at each read head position is varied. To calculate the read head discrimination at position i for a model of length k with alphabet of length n, we define the discrimination at read-head position i as the median of the standard deviations in current level for each of the nk-1 groups of size n where position i is varied while other positions are held constant. Right hand panel demonstrates the base contribution plot: Median current over all sequence contexts with base b (A, T, G or C) at position i of the reader head. A. Complex of CsgG Y51A/F56Q/N91R/K94Q/R97W-del(V105-I107) pore and CsgF (1-35) peptide. B. Complex of CsgG Y51T/N55V/F56Q/N91R/K94Q/R97W-del(V105-I107) pore and CsgF-N175-(1-35). C. Complex of CsgG Y51A/N55V/F56Q/N91R/K94Q/R97W-del(V105-I107) pore and CsgF-N175-(1-35). D. Complex of CsgG Y51T/N55V/F56Q/N91R/K94Q/R97W-del(V105-I107) pore and CsgF-N175-(1-35). E. Complex of CsgG Y51A/N551/F56Q/N91R/K94Q/R97W-del(V105-I107) pore and CsgF-N175-(1-35). F. Complex of CsgG Y51S/N55V/F56Q/N91R/K94Q/R97W-del(V105-I107) pore and CsgF-N175-(1-35). G. Complex of CsgG Y51A/F561/N91R/K94Q/R97W-del(V105-I107) pore and

CsgF-N175-(1-35). F. Complex of CsgG Y51A/F56Q/N91R/K94Q/R97W/R192D-del(V105-I107) pore and CsgF-N17S-(1-45).

FIG. 26 shows the error profiles of the double reader head pore. A. Schematic representation of the CsgG:CsgF complex and the interaction of bases of the DNA with the two reader heads. Red: strong interactions, orange: weak interactions, grey: no interactions. B. Comparison of errors in deletions. Reads from Y51A/F56Q/N91R/K94Q/R97W/R192D-del(V105-I107) and Y51A/N55V/F56Q/N91R/K94Q/R97W-del(V105-I107): CsgF-N175-(1-35) pores were basecalled from the same region of E. coli DNA. Reads were aligned to the reference genome using Minimap2 (arxiv.org/abs/1708.01492), and the resultant alignments were visualised in Savant Genome Browser (ncbi.nlm.nih.gov/pubmed/20562449). The majority of

Y51A/F56Q/N91R/K94Q/R97W/R192D-del(V105-I107) reads contain a single base deletion (black boxes) in the T homopolymer, which is not present in the majority of CsgG:CsgF reads. C. Comparison of the consensus accuracy from unpolished data generated from Y51A/F56Q/N91R/K94Q/R97W/R192D-del(V105-I107) (blue) and Y51A/N55V/F56Q/N91R/K94Q/R97W-del(V105-I107):CsgF-N17S-(1-35) pores (green) against the length of homopolymers.

FIG. 27 shows the homopolymer calling of CsgG:CsgF complex. DNA with the sequence shown in (A) is translocated through the Y51A/F56Q/N91R/K94Q/R97W/R192D-del(V105-I107) pore (B) and the Y51A/N55V/F56Q/N91R/K94Q/R97W-del(V105-I107):CsgF-N175-(1-35) pore (C) and their signal was analysed for the first polyT section shown in light grey in (A). When the polyT section is passing through the CsgG pore which contains a single reader head (model is based on 5 bases located in the reader head), it generates a flat line in the signal. Therefore, it is difficult to determine the exact number of bases in this region which usually causes deletion errors. When the DNA is passing through the CsgG:CsgF complex which contains two reader heads (model is based on 9 bases located within and in between the two reader heads), polyT section shows multiple steps instead of a flat line. Information in these steps can be used to correctly identify the number of bases in the homopolymeric region. This additional information significantly reduce deletion errors and improves overall consensus accuracy.

FIG. 28 shows the characterisation of the CsgG pore (Y51A/F56Q/N91R/K94Q/R97W/-del(V105-I107). A. Reader head discrimination of the CsgG pore. The average variation in modelled current when the base at each read head position is varied. To calculate the read head discrimination at position i for a model of length k with alphabet of length n, we define the discrimination at read-head position i as the median of the standard deviations in current level for each of the nk-1 groups of size n where position i is varied while other positions are held constant. B. Base contribution plot of the CsgG pore. Median current over all kmers with base b (A, T, G or C) at position I of the reader head. C. Current signature when the DNA strand is passing through the CsgG pore.

FIG. 29: Left) Schematic representation of a system according to the present disclosure comprising a nanopore and an auxiliary protein. Both the nanopore and the auxiliary protein contain at least one reader head (constriction region) capable of analyte discrimination, which are represented schematically as the narrowest points in the continuous channel through the complex. Right) Schematic representation of a system comprising a nanopore and an auxiliary protein for the characterisation of polynucleotides, for example for the purposes of sequencing the polynucleotide, where the movement of the polynucleotide through the system is controlled by another entity, most preferably for example a polynucleotide-binding motor enzyme.

FIG. 30: 3D representations of example auxiliary proteins. A) Pentraxin from Limulus polyphemus (pdb=3FLT, 3FLP). B) the oligomeric form of SP1 (pdb=1TR0). C) the oligomeric form of E. coli GroES protein (pdb=1PCQ). The Figures shows the protein viewed from above (top row) and viewed from the side (bottom row). From above the channel through the protein and minimum diameter constrictions are clearly visible. The side views of the proteins are sliced down the central axis to reveal the interiors. The Figures are marked with the approximate inner and outer dimensions of the proteins.

FIG. 31: Interactions between GroES and a single stranded DNA placed within the channel. Data from two different runs show that L49, E50, N51, E53 and Y71 amino acids of GroES (E. coli) interacts with the DNA strand. These positions may be engineered to improve the resolution of the signal.

FIG. 32: Schematic representations of various ways in which an example auxiliary protein (in this case GroES) can be coupled with a nanopore (in this case CsgG) to create different systems with different properties. The figures illustrate how the auxiliary protein can be coupled to either end of the nanopore. For example, for analytes translocating from one side of the membrane to the other this would encounter the two readers in a different order. Likewise, the figure also illustrates that either end of the auxilary protein may be coupled to the nanopore. These variations can be used to control the geometry of the system and the distance between the readers. Although not illustrated, it is possible to combine the scenarios illustrated, for example auxiliary proteins could be coupled to both ends of the nanopore, for example to create a three reader head system. A similar example is shown with the CsgG nanopore and two auxiliary proteins GroES and CsgF in FIGS. 43-45.

FIG. 33: Representation of the pore complex of CsgG with the auxiliary protein FCP (1-36 of CsgF peptide. A) Model representation of the complex from the side view. B)

Visualisation of the channel through the pore complex. C) Pore radius profile of the pore complex showing the pore radius of the channel through the CsgG-FCP protein complex.

FIG. 34: Representation of the pore complex of MspA (PDB=1UUN) and GroES (PDB=1PCQ). A) Model representation of the complex from the side view. GroES auxiliary protein was placed on top of the MspA nanopore such that the distance between the proteins was minimised. B) Visualisation of the channel through the pore complex. C) Pore radius profile of the pore complex showing the radius of the channel through the MspA-GroES protein complex.

FIG. 35: Representation of the pore complex of MspA (PDB=1UUN) and SP1 (PDB=1TRO). A) Model representation of the complex from the side view. SP1 auxiliary protein was placed on top of the MspA nanopore such that the distance between the proteins was minimised. B) Visualisation of the channel through the pore complex. C) Pore radius profile of the pore complex showing the radius of the channel through the MspA-SP1 protein complex.

FIG. 36: Representation of the pore complex of MspA (PDB=1UUN) and Pentraxin (PDB=3FLP). A) Model representation of the complex from the side view. Pentraxin auxiliary protein was placed on top of the MspA nanopore such that the distance between the proteins was minimised. B) Visualisation of the channel through the pore complex. C) Pore radius profile of the pore complex showing the radius of the channel through the MspA-Pentraxin protein complex.

FIG. 37: Representation of the pore complex of alpha-hemolysin (PDB=7AHL) and GroES (PDB=1PCQ). A) Model representation of the complex from the side view. GroES auxiliary protein was placed on top of the alpha-hemolysin nanopore such that the distance between the proteins was minimised. B) Visualisation of the channel through the pore complex. C) Pore radius profile of the pore complex showing the radius of the channel through the alpha-hemolysin-GroES protein complex.

FIG. 38: Representation of the pore complex of alpha-hemolysin (PDB=7AHL) and SP1 (PDB=1TRO). A) Model representation of the complex from the side view. SP1 auxiliary protein was placed on top of the alpha-hemolysin nanopore such that the distance between the proteins was minimised. B) Visualisation of the channel through the pore complex. C) Pore radius profile of the pore complex showing the radius of the channel through the alpha-hemolysin-SP1 protein complex.

FIG. 39: Representation of the pore complex of alpha-hemolysin (PDB=7AHL) and Pentraxin (PDB=3FLP). A) Model representation of the complex from the side view. SP1 auxiliary protein was placed on top of the alpha-hemolysin nanopore such that the distance between the proteins was minimised. B) Visualisation of the channel through the pore complex. C) Pore radius profile of the pore complex showing the radius of the channel through the alpha-hemolysin-Pentraxin protein complex.

FIG. 40: Representation of the pore complex of CsgG (PDB=4UV3) and GroES (PDB=1PCQ). A) Model representation of the complex from the side view. GroES auxiliary protein was placed on top of the CsgG nanopore such that the distance between the proteins was minimised. B) Visualisation of the channel through the pore complex. C) Pore radius profile of the pore complex showing the radius of the channel through the CsgG-GroES protein complex.

FIG. 41: Representation of the nanopore complex of CsgG (PDB=4UV3) and SP1 (PDB=1TRO). A) Model representation of the complex from the side view. SP1 auxiliary protein was placed on top of the CsgG pore such that the distance between the proteins was minimised. B) Visualisation of the channel through the pore complex. C) Pore radius profile of the pore complex showing the radius of the channel through the CsgG-SP1 protein complex.

FIG. 42: Representation of the pore complex of CsgG (PDB=4UV3) and Pentraxin (PDB=3FLP). A) Model representation of the complex from the side view. SP1 auxiliary protein was placed on top of the CsgG nanopore such that the distance between the proteins was minimised. B) Visualisation of the channel through the pore complex. C) Pore radius profile of the pore complex showing the radius of the channel through the CsgG-Pentraxin protein complex.

FIG. 43: Representation of the pore complex of CsgG with the auxiliary proteins FCP (1-36 of CsgF peptide) and GroES (PDB=1PCQ). A) Model representation of the complex from the side view. GroES auxiliary protein was placed on top of the CsgG-FCP complex such that the distance between the proteins was minimised. B) Visualisation of the channel through the pore complex. C) Pore radius profile of the pore complex showing the radius of the channel through the CsgG-FCP-GroES protein complex.

FIG. 44: Representation of the pore complex of CsgG with the auxiliary proteins FCP (1-36 of CsgF peptide) and SP1 (PDB=1TRO). A) Model representation of the complex from the side view. GroES auxiliary protein was placed on top of the CsgG-FCP complex such that the distance between the proteins was minimised. B) Visualisation of the channel through the pore complex. C) Pore radius profile of the pore complex showing the radius of the channel through the CsgG-FCP-SP1 protein complex.

FIG. 45: Representation of the pore complex of CsgG with the auxiliary proteins FCP (1-36 of CsgF peptide) and Pentraxin (PDB=3FLP). A) Model representation of the complex from the side view. GroES auxiliary protein was placed on top of the CsgG-FCP complex such that the distance between the proteins was minimised. B) Visualisation of the channel through the pore complex. C) Pore radius profile of the pore complex showing the radius of the channel through the CsgG-FCP-Pentraxin protein complex.

FIG. 46: Pore radius profiles of the MspA nanopore and GroES auxiliary proteins from E. coli (PDB=1PCQ) and Thermus thermophilus (PDB=1WNR). The data show that the dimensions of the constriction region of GroES are comparable with the dimensions of the constriction region of the MspA nanopore.

FIG. 47: A schematic representation of a single stranded DNA molecule placed within the channel of GroES (PDB=1PCQ).

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein” includes two or more such proteins, reference to “a helicase” includes two or more helicases, reference to “a monomer” refers to two or more monomers, reference to “a pore” includes two or more pores and the like.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Standard substitution notation is also used, i.e. Q42R means that Q at position 42 is replaced with R.

In the paragraphs herein where different amino acids at a specific position are separated by the / symbol, the / symbol means “or”. For instance, Q87R/K means Q87R or Q87K.

In the paragraphs herein where different positions are separated by the / symbol, the / symbol means “and” such that Y51/N55 is Y51 and N55.

All amino-acid substitutions, deletions and/or additions disclosed herein are with reference to a mutant CsgG monomer comprising a variant of the sequence shown in SEQ ID NO: 3, unless stated to the contrary.

Reference to a mutant CsgG monomer comprising a variant of the sequence shown in SEQ ID NO: 3 encompasses mutant CsgG monomers comprising variants of sequences. Amino-acid substitutions, deletions and/or additions may be made to CsgG monomers comprising a variant of the sequence other than shown in SEQ ID NO: 3 that are equivalent to those substitutions, deletions and/or additions disclosed herein with reference to a mutant CsgG monomer comprising a variant of the sequence shown in SEQ ID NO: 3.

All publications, patents and patent applications cited herein, whether supra or infra, are hereby incorporated by reference in their entirety.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

“Gene” as used here includes both the promoter region of the gene as well as the coding sequence. It refers both to the genomic sequence (including possible introns) as well as to the cDNA derived from the spliced messenger, operably linked to a promoter sequence.

“Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cy s; D=Asp; E=Glu; F=Phe; G=Gly; H=His; I=Ile; K=Ly s; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

Polypeptides can also undergo maturation or post-translational modification processes that may include, but are not limited to: glycosylation, proteolytic cleavage, lipidization, signal peptide cleavage, propeptide cleavage, phosphorylation, and such like. By “recombinant polypeptide” is meant a polypeptide made using recombinant techniques, e.g., through the expression of a recombinant or synthetic polynucleotide. When the chimeric polypeptide or biologically active portion thereof is recombinantly produced, it is also preferably substantially free of culture medium, e.g., culture medium represents less than about 20%, more preferably less than about 10%, and most preferably less than about 5% of the volume of the protein preparation. By “isolated” is meant material that is substantially or essentially free from components that normally accompany it in its native state. For example, an “isolated polypeptide”, as used herein, refers to a polypeptide, which has been purified from the molecules which flank it in a naturally-occurring state, e.g., a CsgF peptide which has been removed from the molecules present in the production host that are adjacent to said polypeptide. An isolated peptide can be generated by amino acid chemical synthesis or can be generated by recombinant production. An isolated complex can be generated by in vitro reconstitution after purification of the components of the complex, e.g. a CsgG pore and the CsgF peptide(s), or can be generated by recombinant co-expression.

The term “protein” is used to describe a folded polypeptide having a secondary or tertiary structure. The protein may be composed of a single polypeptide, or may comprise multiple polypepties that are assembled to form a multimer. The multimer may be a homooligomer, or a heteroligmer. The protein may be a naturally occurring, or wild type protein, or a modified, or non-naturally, occurring protein. The protein may, for example, differ from a wild type protein by the addition, substitution or deletion of one or more amino acids.

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

“Variant”, “Homologue” and “Homologues” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The term “transmembrane protein pore” defines a pore comprising multiple pore monomers. Each momomer may be a wild-type monomer, or a variant of thereof. The variant momomer may also be referred to as a modified monomer or a mutant monomer. The modifications, or mutations, in the variant include but are not limited to any one or more of the modifications disclosed herein, or combinations of said modifications.

The term “CsgG pore” defines a pore comprising multiple CsgG monomers. Each CsgG momomer may be a wild-type monomer from E. coli (SEQ ID NO: 3), wild-type homologues of E. coli CsgG, such as for example, monomers having any one of the amino acid sequences shown in SEQ ID NOS: 68 to 88, or a variant of any thereof (e.g. a variant of any one of SEQ ID NOs: 3 and 68 to 88). The variant CsgG momomer may also be referred to as a modified CsgG monomer or a mutant CsgG monomer. The modifications, or mutations, in the variant include but are not limited to any one or more of the modifications disclosed herein, or combinations of said modifications.

For all aspects and embodiments of the present invention, a homologue is referred to as a polypeptide that has at least 50%, 60%, 70%, 80%, 90%, 95% or 99% complete sequence identity to the amino acid sequence of the corresponding wild-type protein. For example, a CsgG homologue has at least 50%, 60%, 70%, 80%, 90%, 95% or 99% complete sequence identity to E. coli CsgG as shown in SEQ ID NO: 3. A CsgG homologue is also referred to as a polypeptide that contains the PFAM domain PF03783, which is characteristic for CsgG-like proteins. A list of presently known CsgG homologues and CsgG architectures can be found at pfam.xfam.org//family/PF03783. Likewise, a homologous polynucleotide can comprise a polynucleotide that has at least 50%, 60%, 70%, 80%, 90%, 95% or 99% complete sequence identity to the nucleic acid sequence encoding a wild-type protein. For example, a CsgG homologous polynucleotide can comprise a polynucleotide that has at least 50%, 60%, 70%, 80%, 90%, 95% or 99% complete sequence identity to E. coli CsgG as shown in SEQ ID NO: 1.

Examples of homologues of CsgG shown in SEQ ID NO:3 have the sequences shown in SEQ ID NOS: 68 to 88.

The term “modified CsgF peptide” or “CsgF peptide” defines a CsgF peptide that has been truncated from its C-terminal end (e.g. is an N-terminal fragment) and/or is modified to include a cleavage site. The CsgF peptide may be a fragment of wild-type E. coli CsgF (SEQ ID NO: 5 or SEQ ID NO: 6), or of a wild-type homologue of E. coli CsgF, such as for example, a peptide comprising any one of the amino acid sequences shown in SEQ ID NOS: 17 to 36, or a variant (e.g. one modified to include a cleavage site) of any thereof.

For all aspects and embodiments of the present invention, a CsgF homologue is referred to as a polypeptide that has at least 50%, 60%, 70%, 80%, 90%, 95% or 99% complete sequence identity to wild-type E. coli CsgF as shown in SEQ ID NO: 6. In some embodiments, a CsgF homologue is also referred to as a polypeptide that contains the PFAM domain PF10614, which is characteristic for CsgF-like proteins. A list of presently known CsgF homologues and CsgF architectures can be found at pfam.xfam.org//family/PF10614. Likewise, a CsgF homologous polynucleotide can comprise a polynucleotide that has at least 50%, 60%, 70%, 80%, 90%, 95% or 99% complete sequence identity to wild-type E. coli CsgF as shown in SEQ ID NO: 4. Examples of truncated regions of homologues of CsgF shown in SEQ ID NO: 6 have the sequences shown in SEQ ID NOs:17 to 36.

The term “N-terminal portion of a CsgF mature peptide” refers to a peptide having an amino acid sequence that corresponds to the first 60, 50, or 40 amino acid residues starting from the N-terminus of a CsgF mature peptide (without a signal sequence). The CsgF mature peptide can be a wild-type or mutant (e.g., with one or more mutations).

Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence. Homology to the nucleic acid sequence of SEQ ID NO: 1 for CsgG homologues or SEQ ID NO:4 for CsgF homologues, respectively, is not limited simply to sequence identity. Many nucleic acid sequences can demonstrate biologically significant homology to each other despite having apparently low sequence identity. Homologous nucleic acid sequences are considered to be those that will hybridise to each other under conditions of low stringency (M. R. Green, J. Sambrook, 2012, Molecular Cloning: A Laboratory Manual, Fourth Edition, Books 1-3, Cold Spring Harbor Laboratory Press, Cold Spring Harbor, N.Y.).

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

Proteins can also be fusion proteins, referring in particular to genetic fusion, made e.g., by recombinant DNA technology. Proteins can also be conjugated, or “conjugated to”, as used herein, which refers, in particular, to chemical and/or enzymatic conjugation resulting in a stable covalent link. For example, two, more or all of the polypeptide subunits of a multimeric auxiliary protein and/or nanopore may be fused, and/or a polypeptide subunit of an auxiliary protein may be fused to a monomer of the nanopore.

Proteins may form a protein complex when several polypeptides or protein monomers bind to or interact with each other. “Binding” means any interaction, be it direct or indirect. A direct interaction implies a contact between the binding partners, for instance through a covalent link or coupling. An indirect interaction means any interaction whereby the interaction partners interact in a complex of more than two compounds. The interaction can be completely indirect, with the help of one or more bridging molecules, or partly indirect, where there is still a direct contact between the partners, which is stabilized by the additional interaction of one or more compounds. The “complex” as referred to in this disclosure is defined as a group of two or more associated proteins, which might have different functions. The association between the different polypeptides of the protein complex might be via non-covalent interactions, such as hydrophobic or ionic forces, or may as well be a covalent binding or coupling, such as disulphide bridges, or peptidic bonds. Covalent “binding” or “coupling” are used interchangeably herein, and may also involve “cysteine coupling” or “reactive or photoreactive amino acid coupling”, referring to a bioconjugation between cysteines or between (photo)reactive amino acids, respectively, which is a chemical covalent link to form a stable complex. Examples of photoreactive amino acids include azidohomoalanine, homopropargylglycyine, homoallelglycine, p-acetyl-Phe, p-azido-Phe, p-propargyloxy-Phe and p-benzoyl-Phe (Wang et al. 2012, in Protein Engineering, DOI: 10.5772/28719; Chin et al. 2002, Proc. Nat. Acad. Sci. USA 99(17); 11020-24).

A “transmembrane protein pore” or “biological pore” is a transmembrane protein structure defining a channel or hole that allows the translocation of molecules and ions from one side of the membrane to the other. The translocation of ionic species through the pore may be driven by an electrical potential difference applied to either side of the pore. A “nanopore” is a pore in which the minimum diameter of the channel through which molecules or ions pass is in the order of nanometres (10⁻⁹ metres). The minimum diameter is the diameter at the narrowest point of the constriction. The transmembrane protein pore may be monomeric or oligomeric in nature. Typically, the pore comprises a plurality of polypeptide subunits arranged around a central axis thereby forming a protein-lined channel that extends substantially perpendicular to the membrane in which the nanopore resides. The number of polypeptide subunits is not limited. Typically, the number of subunits is from 5 to up to 30, suitably the number of subunits is from 6 to 10. Alternatively, the number of subunits is not defined as in the case of perfringolysin or related large membrane pores. The portions of the protein subunits within the nanopore that form protein-lined channel typically comprise secondary structural motifs that may include one or more trans-membrane β-barrel, and/or α-helix sections.

The term “pore complex” refers to an oligomeric pore, wherein a nanopore and an auxiliary protein or peptide are associated in the complex and together form a continuous channel that has two constriction regions. When the pore complex is provided in an environment having membrane components, membranes, cells, or an insulating layer, the pore complex will insert in the membrane or the insulating layer, and form a “transmembrane pore complex”.

The pore complex or transmembrane pore complex of the disclosure is suited for analyte characterization. In some embodiments, the pore complex or transmembrane complex described herein can be used for sequencing polynucleotide sequences e.g., because it can discriminate between different nucleotides with a high degree of sensitivity. The pore complex of the disclosure may be an isolated pore complex, substantially isolated, purified or substantially purified. A pore complex of the disclosure is “isolated” or purified if it is completely free of any other components, such as lipids and/or other pores, or other proteins with which it is normally associated in its native state e.g., for CsgG and/or CsgF, CsgE, CsgA CsgB, or if it is sufficiently enriched from a membranous compartment. A pore complex is substantially isolated if it is mixed with carriers or diluents which will not interfere with its intended use. For instance, a pore complex is substantially isolated or substantially purified if it is present in a form that comprises less than 10%, less than 5%, less than 2% or less than 1% of other components, such as triblock copolymers, lipids or other pores. Alternatively, a pore complex of the disclosure may be a transmembrane pore complex, when present in a membrane.

The “constriction”, “orifice”, “constriction region”, “channel constriction”, “constriction site”, or “reader head” as used interchangeably herein, refers to an aperture defined by a luminal surface of a pore or pore complex, which acts to allow the passage of ions and target molecules (e.g., but not limited to polynucleotides or individual nucleotides) but not other non-target molecules through the pore channel or continuous channel formed by the pore and auxiliary protein or peptide. In some embodiments, the constriction(s) are the narrowest aperture(s) within a pore or pore complex. In this embodiment, the constriction(s) may serve to limit the passage of molecules through the pore. The size of the constriction is typically a key factor in determining suitability of a nanopore for nucleic acid sequencing applications. If the constriction is too small, the molecule to be sequenced will not be able to pass through. However, to achieve a maximal effect on ion flow through the channel, the constriction should not be too large. For example, the constriction should preferably not be wider than the solvent-accessible transverse diameter of a target analyte. Ideally, any constriction should be as close as possible in diameter to the transverse diameter of the analyte passing through. For sequencing of nucleic acids and nucleic acid bases, suitable constriction diameters are in the nanometre range (10⁻⁹ meter range). Suitably, the diameter should be in the region of 0.5 to 2.0 nm, or 0.5 to 4.0 nm, typically, the diameter is in the region of 0.7 to 1.2 nm, such as 0.9 nm (9 Å). Such diameters may be particularly suited for sequencing of single-stranded nucleic acids. Larger diameters, such as from about 1.2 nm to about 4 nm, such as about 2 to about 4 nm or about 3 nm to about 4 nm may be particularly suited for sequencing of double-stranded nucleic acids.

When two or more constrictions are present and spaced apart each constriction may interact with or “read” separate nucleotides within the nucleic acid strand at the same time. In this situation, the reduction in ion flow through the channel will be the result of the combined restriction in flow of all the constrictions containing nucleotides. Hence, in some instances a double constriction may lead to a composite current signal. In certain circumstances, the current read-out for one constriction, or “reading head”, may not be able to be determined individually when two such reading heads are present. The additional channel constriction or reader head provided by the auxiliary protein or peptide may be positioned about 15 nm or less, such as about 12 nm or less, about 11 nm or less, about 10 nm or less, or about 5 nm or less, or about 1, 2, 3, 4, 5, 6, 7, 8, 9, 10, 11, 12, 13, 14 or 15 nm, from the constriction region of the nanopore. The pore complex or transmembrane pore complex of the disclosure includes pore complexes with two reader heads, meaning, channel constrictions positioned in such a way to provide a suitable separate reader head without interfering the accuracy of other constriction channel reader heads.

A constriction region or constriction site may be formed by one or more specific amino acid residues within the protein sequence of a transmembrane protein nanopore and/or an auxiliary protein or peptide.

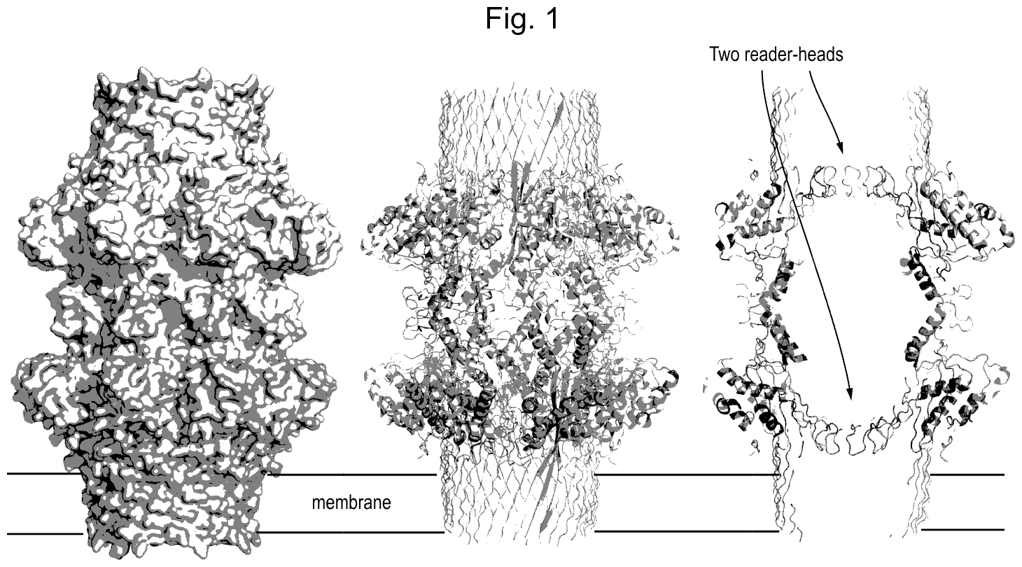

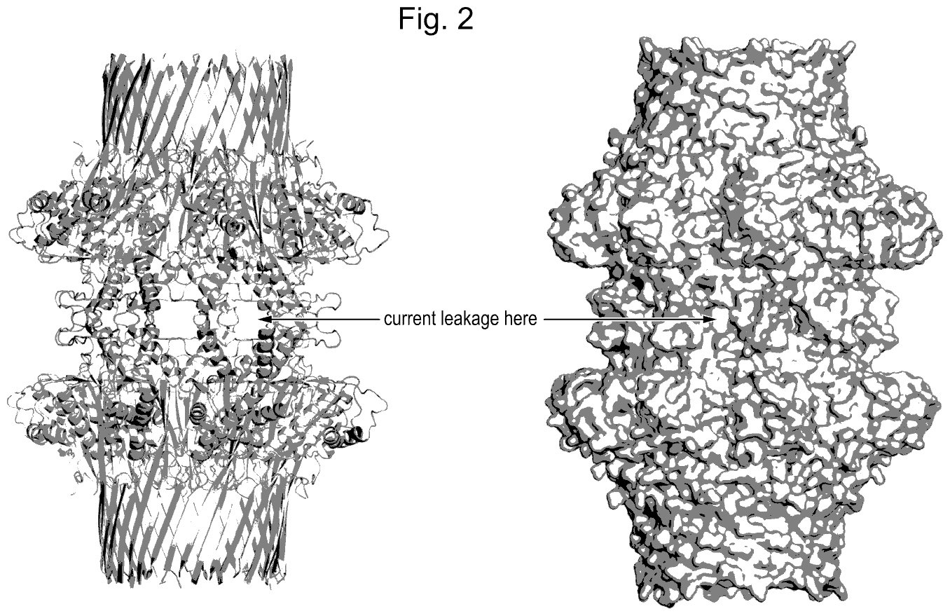

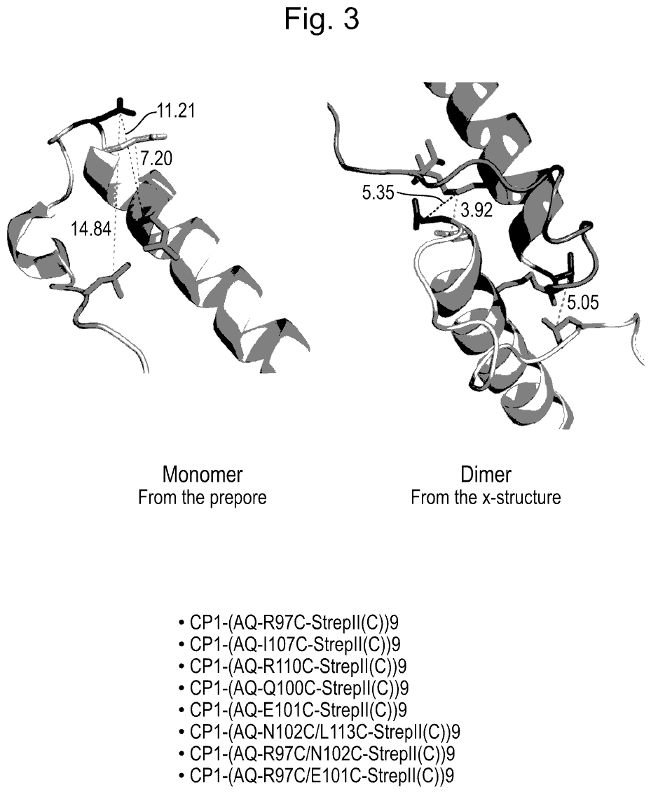

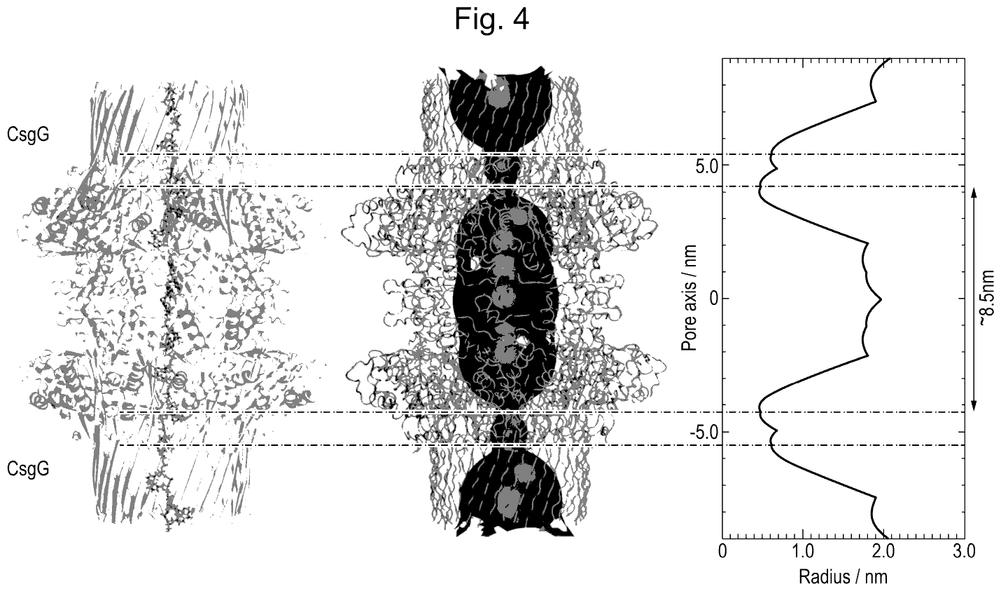

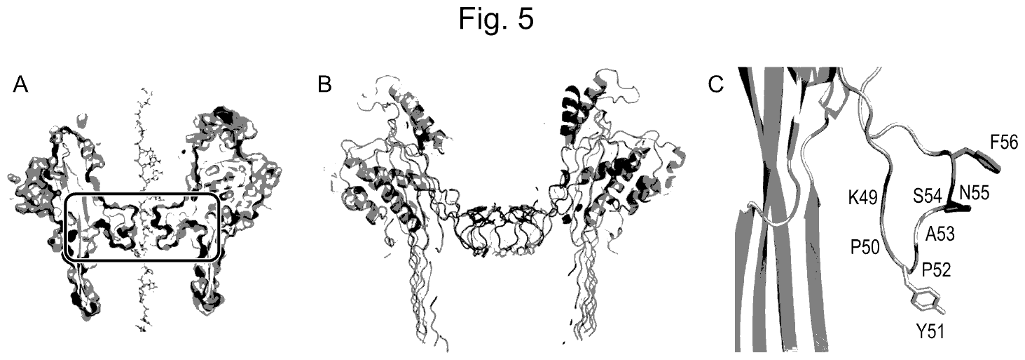

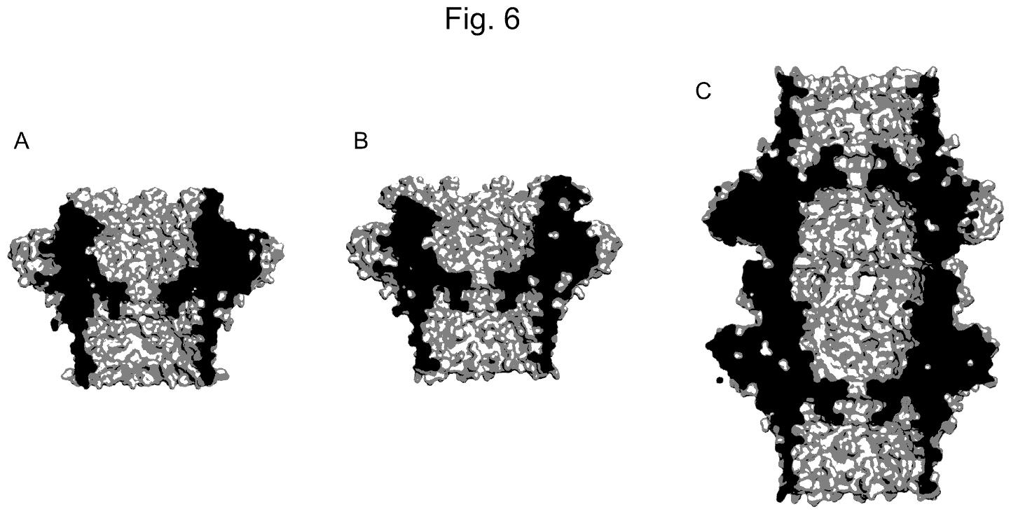

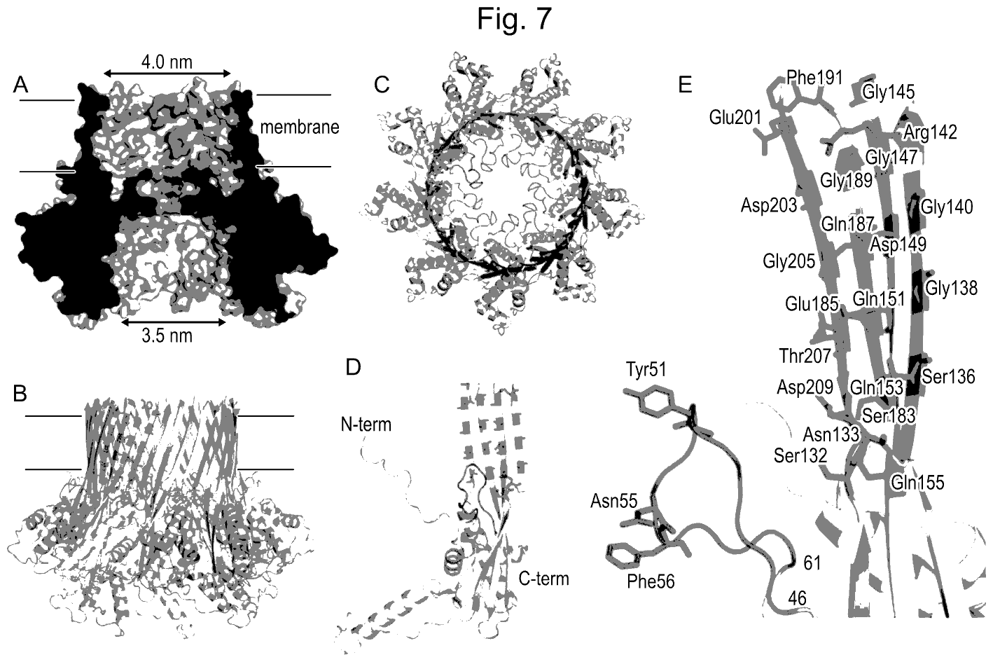

The constriction of wild type E. coli CsgG (SEQ ID NO:3), for example, is composed of two annular rings formed by juxtaposition of tyrosine residues at position 51 (Tyr 51) in the adjacent protein monomers, and also the phenylalanine and asparagine residues at positions 56 and 55 respectively (Phe 56 and Asn 55) (FIG. 1). The wild-type pore structure of CsgG is in most cases being re-engineered via recombinant genetic techniques to widen, alter, or remove one of the two annular rings that make up the CsgG constriction (mentioned as “CsgG channel constriction” herein), to leave a single well-defined reading head. The constriction motif in the CsgG oligomeric pore is located at amino acid residues at position 38 to 63 in the wild type monomeric E. coli CsgG polypeptide, depicted in SEQ ID NO: 3. In considering this region, mutations at any of the amino acid residue positions 50 to 53, 54 to 56 and 58 to 59, as well as key of positioning of the sidechains of Tyr51, Asn55, and Phe56 within the channel of the wild-type CsgG structure, was shown to be advantageous in order to modify or alter the characteristics of the reading head. The present disclosure relating to a pore complex comprising a CsgG-pore and a modified CsgF peptide, or homologues or mutants thereof, surprisingly added another constriction (mentioned as “CsgF channel constriction” herein) to the CsgG-containing pore complex, forming a suitable additional, second reader head in the pore, via complex formation with the modified CsgF peptide. Said additional CsgF channel constriction or reader head is positioned adjacent to the constriction loop of the CsgG pore, or of the mutated CsgG pore. Said additional CsgF channel constriction or reader head is positioned approximately 10 nm or less, such as 5 nm or less, such as 1, 2, 3, 4, 5, 6, 7, 8, 9 nm from the constriction loop of the CsgG pore, or of the mutated CsgG pore. The pore complex or transmembrane pore complex of the disclosure includes pore complexes with two reader heads, meaning, channel constrictions positioned in such a way to provide a suitable separate reader head without interfering the accuracy of other constriction channel reader heads. Said pore complexes therefore may include CsgG mutant pores (see incorporated references WO2016/034591, WO2017/149316, WO2017/149317, WO2017/149318 and International patent application no. PCT/GB2018/051191 each of which lists mutations to the wild-type CsgG pore that improve the properties of the pore) as well as wild-type CsgG pores, or homologues thereof, together with a modified CsgF peptide, or homologue or mutant thereof, wherein said CsgF peptide has another constriction channel forming a reader head.

### Pore Complex

The disclosure relates to nanopores complexed with an auxiliary protein or peptide to produce a channel having at least two constrictions. In one embodiment the pore complex comprises: (i) a nanopore located in the membrane, and (ii) an auxiliary protein or peptide attached to the nanopore, wherein the nanopore and the auxiliary protein or peptide together form a continuous channel across the membrane, the channel comprising a first constriction region and a second constriction region, and wherein the first constriction region is formed by a portion of the nanopore, and wherein the second constriction region is formed by at least a portion of the auxiliary protein or peptide.

The continuous channel typically provides a passage through which a polynucleotide can pass. For example, the channel can accommodate a polynucleotide, wherein one end of the polynucleotide is directed towards or extends out of one end of the channel and the other end of the polynucleotide is directed towards or extends out of the other end of the channel. Where the pore complex is located in a membrane, the continuous channel is suitable for translocation of a polynucleotide across the membrane.

All or part of the auxiliary protein or peptide may be located within the lumen of the nanopore. In this embodiment, the constriction formed by the auxiliary protein or peptide may be inside or outside the part of the lumen of the nanopore, or at the entrance to the lumen of the nanopore. Alternately, the auxiliary protein or peptide, and hence the constriction formed by the auxiliary protein or peptide may be located entirely outside the lumen of the nanopore. Where all or part of the auxiliary protein or peptide is located outside the lumen of the nanopore, it may extend from or be adjacent to either side of the nanopore. The pore complex may comprise a first auxiliary protein or peptide located on one side of the nanopore and a second auxiliary protein or peptide located on the same side, or on the other side of the nanopore such that the two auxiliary proteins or peptides and the nanopore together define a continuous channel. The first and second auxiliary proteins or peptides may be the same or different. Where the pore complex is present in a membrane having a cis side and a trans side, the auxiliary protein or peptide may be located on the cis side of the membrane or on the trans side of the membrane.

The auxiliary protein or peptide and nanopore may be configured in the complex, such that each interacting nucleotide of polynucleotide translocating through the continuous channel first interacts with the constriction region formed by the nanopore and then with the constriction region formed by the auxiliary protein or peptide. For example, wherein the polynucleotide passes from the cis side of a membrane to the trans side, the constriction region formed by the nanopore is located in the continuous channel at a position closer to the cis side of the membrane than the constriction region formed by the auxiliary protein or peptide.

Alternatively, the auxiliary protein or peptide and nanopore may be configured in the complex, such that each interacting nucleotide of polynucleotide translocating through the continuous channel first interacts with the constriction region formed by the auxiliary protein or peptide and then with the constriction region formed by the nanopore. For example, wherein the polynucleotide passes from the cis side of a membrane to the trans side, the constriction region formed by the auxiliary protein or peptide is located in the continuous channel at a position closer to the cis side of the membrane than the constriction region formed by the nanopore.

Where the auxiliary protein or peptide is located outside the pore, the auxiliary protein or peptide itself typically has a central aperture that forms part of the continuous channel in the pore complex, and includes a constriction region. In other words, the auxiliary protein or peptide may be ring-shaped. A ring-shaped auxiliary protein or peptide may in some embodiments be located inside, or partially inside, the lumen of the nanopore.

Where the auxiliary protein or peptide is located at least partially inside the pore, the auxiliary protein or peptide itself may or may not contain a central aperture that forms part of the continuous channel in the pore complex, and includes a constriction region. In other words, the auxiliary protein or peptide may be ring-shaped. Alternatively, the constriction region may be formed only when the auxiliary protein or peptide interacts with the nanopore. For example, the auxiliary peptide may interact with the nanopore to constrict the lumen of the nanopore and hence form a constriction in the channel. In one embodiment, the pore complex may comprise multiple molecules of the peptide, wherein each interacts with one monomer of a protein nanopore, thus producing a concentric ring of peptides forming a constriction.

In one embodiment, the complex comprises two or more auxiliary proteins or peptides, wherein each auxiliary protein or peptide forms part of the lumen of a channel continuous with the channel of a nanopore and each forms a constriction. In this embodiment, the nanopore may or may not contain a constriction. In one form of this embodiment, a first auxiliary protein or peptide may be located on one side of the nanopore and a second auxiliary protein or peptide may be located on the other side of the nanopore such that the two auxiliary proteins or peptides and the nanopore together define a continuous channel. The first and second auxiliary proteins or peptides may be the same or different.

In one embodiment, a constriction region may have a minimum diameter of about 0.5 to about 4.0 nanometres, such as from about 0.5 to about 3.0 nanometres or about 0.5 to about 2.0 nanometres, preferably about 0.7 to about 1.8 nanometres, about 0.8 to about 1.7 nanometres, about 0.9 to about 1.6 nanometres, or about 1.0 to about 1.5 nanometres, such as about 1.1, 1.2, 1.3 or 1.4 nanometres. The two or more constriction regions in the channel of the pore complex may have the same minimum diameter, or the two channels may have different minimum diameters. The length of a constriction region may be such that only one nucleotide in a polynucleotide located in the channel influences the current flowing through the pore complex, or such that 2 or more, such as 3, 4, 5, 6 or 7 nucleotides in the polynucleotide influence the current. The lengths of the two constrictions may also be the same, similar or different. For example, one of two constrictions in a pore complex may result in a signal that is influenced by 1 or 2 nucleotides, and the other constriction may give rise to a signal that is influenced by 4 or 5 nucleotides. Thus, one constriction may serve as a sharp reader head, and the other as a broad reader head.

The diameter of a constriction region may vary over the length of the constriction. In one embodiment, the constriction region may be defined as a region of a pore that has a diameter ranging from about 0.5 to about 4.0 nanometres, such as from about 0.5 to about 2.0 nanometres, preferably about 0.7 to about 1.8 nanometres, about 0.8 to about 1.7 nanometres, about 0.9 to about 1.6 nanometres, or about 1.0 to about 1.5 nanometres, such as about 1.1, 1.2, 1.3 or 1.4 nanometres.In one embodiment, the distance along the length of the channel between a first constriction region and a second constriction region is from about 1 to about 10 nanometres, or about 2 to about 10 nanometres, for example from about 2 to about 9 nanometres, about 3 to about 8 nanometres, about 4 to about 7 nanometres; or about 1, about 2, about 3, about 4, about 5, about 6, about 7, about 8, about 9, or about 10 nanometres.

In one embodiment, each of the first and second constriction regions is capable of discriminating between different nucleotides of a polynucleotide. Thus, when an ionic current is passed through the pore and a polynucleotide is present in the channel, the current blockade, or signal, that results from the interaction of the polynucleotide with a constriction region indicates which nucleotide, or nucleotides, is, or are, interacting with the constriction region. The current blockade, or signal, is typically influenced by the simultaneous interactions of different parts of the polynucleotide with each of the first and second constriction regions.

The additional constriction introduced in the nanopore channel by complex formation with the auxiliary protein or peptide expands the contact surface with passing nucleotides (or other analytes) and can act as a second reader head for nucleotide (or other analyte) detection and characterization. Pore complexes comprising a nanopore combined with an auxiliary protein or peptide can improve the characterisation of polynucleotides, providing a more discriminating direct relationship between the observed current as the polynucleotide moves through the pore. In particular, by having two stacked reader heads spaced at a defined distance, the pore complex may facilitate characterization of polynucleotides that contain at least one homopolymeric stretch, e.g., several consecutive copies of the same nucleotide that otherwise exceed the interaction length of the single nanopore reader head.

Additionally, by having two stacked constrictions at a defined distance, small molecule analytes including organic or inorganic drugs and pollutants passing through the complex pore will consecutively pass two independent reader heads. The chemical nature of either reader head can be independently modified, each giving unique interaction properties with the analyte, thus providing additional discriminating power during analyte detection.

### Auxiliary Protein

In one embodiment, the auxiliary protein may be ring-shaped. In one embodiment, the ring-shaped protein comprises multiple subunits, or monomers, arranged around a central cavity or aperture. In the pore complex, the central cavity, or aperture, is lined up with the lumen of the nanopore to form a continuous channel.

The narrowest point of the central cavity or aperture typically forms a constriction in the continuous channel. The minimum diameter of the constriction may be from about 0.5 nm to about 4.0 nanometres, such as about 0.5 to aboit 3.0 nanometres or about 0.5 to about 2.0 nanometres, preferably from about 0.7 to about 1.8 nanometres, from about 0.8 to about 1.7 nanometres, from about 0.9 to about 1.6 nanometres, or from about 1.0 to about 1.5 nanometres, such as about 1.1, 1.2, 1.3 or 1.4 nanometres. The outer diameter of the ring-shaped protein can be greater or smaller, or approximately the same as the outer diameter of the nanopore. For example, the ring-shaped protein may have a maximum outer diameter of from about 2 nm to about 20 nm, such as from about 5 nm to about 10 nm or about 5 nm to about 15 nm, for example 6 nm to 9 nm or 7 nm to 8 nm. The auxiliary protein may, in some embodiments, be modified from its natural state to provide a constriction having the desired minimum diameter. For example, the auxiliary protein may have a wider than desired internal diameter that is modified, such as by introducing one or more bulky residues by targeted mutation to create a constriction having a minimum diameter within the ranges specified above. The maximum height of the auxiliary protein is in one embodiment, from about 3 nm to about 20 nm, such as from about 4 nm to about 10 nm. In one embodiment, the length of the channel in the auxiliary protein is from about 3 nm to about 20 nm, such as from about 4 nm to about 10 nm. The height is the dimension of the auxiliary protein in a direction perpendicular to the membrane.

The ring-shaped auxiliary protein may have the same symmetry as the nanopore. For example, where the nanopore comprises eight monomers around a central axis, the auxiliary protein preferably has eight-fold symmetry (i.e. comprises eight monomers around a central axis) or where the nanopore comprises nine monomers around a central axis, the auxiliary protein preferably has nine-fold symmetry (i.e. has nine subunits around a central axis) etc. Alternatively, the ring-shaped auxiliary protein may comprise more or fewer, such as one more or one fewer, monomers than the nanopore.

The auxiliary protein typically comprises one or more positively charged amino acids, such as arginine, lysine or histidine, or aromatic amino acids, such as tyrosine or tryptophan within the central cavity, or aperture, such as at, or close to (e.g. within about 1, 2, 3, 4 or 5 nm of the constriction), the constriction. These amino acids typically facilitate the interaction between the pore and polynucleotides.

The auxiliary protein or peptide may be selected from GroES, CsgF, pentraxin, or SP1. The auxiliary protein or peptide may be an inactive lambda exonuclease, or an inactive protease such as Zn-dependent D-aminopeptidase DppA from Bacillus subtilis, AAA+ ring of HslUV protease, or Lon protease from E. coli.

In one embodiment, the auxiliary protein or peptide is not CsgF or a CsgF peptide or a functional homologue, fragment or modified version thereof. In one embodiment, the auxiliary protein or peptide is not a CsgG nanopore, or a homologue, fragment or modified version thereof.

In one embodiment, the auxiliary protein is pentraxin, also known as pentaxin. Pentraxins are a superfamily of multifunctional conserved proteins that comprise a pentraxin protein domain. Pentraxins are ring-shaped multimeric proteins typically formed from 5 or more monomers. Pentraxins typically have a distinctive flattened β-jellyroll structure. Examples of pentraxins include Serum Amyloid P component (SAP), C reactive protein (CRP), female protein (FP), neural pentraxin I (NPTXI), neural pentraxin II (NPTXII), NPTXR, apexin, pentraxin 3 (PTX3) (also known as TNF-inducible gene 14 protein (TSG-14)), G-protein coupled receptor 144 (GPR144) and SVEP1. An example pentraxin amino acid sequence is described in the UniProt database under reference Q8WQK3. In one embodiment, a pentraxin protein may comprise an amino acid sequence of one monomer as set forth in UniProt reference Q8WQK3.

In one embodiment, the auxiliary protein is GroES. GroES is a protein homologous to Heat shock 10 kDa protein 1 (Hsp10), also known as chaperonin 10 (cpn10) or early-pregnancy factor (EPF) in humans. GroES is known in organisms including E. coli. The pore complex may comprise GroES, or a homologue, or modified version, such as a fragment, thereof. The modified version or fragment may be a modified version or fragment of a homologue of GroES. GroES is a ring-shaped homooligomer comprising between six and eight identical subunits. The modified version or fragment has a ring-shape, and typically comprises one or more, preferably from six to eight, modified or truncated subunits. An example GroES amino acid sequence for E. coli GroES is described in the UniProt database under reference P0A6F9. In one embodiment, a GroES protein may comprise an amino acid sequence of one monomer as set forth in UniProt reference P0A6F9.

In one embodiment, the auxiliary protein is Stable Protein 1 (SP1). SP1 may consist of 12 monomers, which may be identical, which form a ring protein complex. An example SP1 amino acid sequence is described in the UniProt database under reference Q9AR79. An SP1 protein may comprise an amino acid sequence of one monomer of 108 amino acid residues as denoted by GenBank Accession No. AJ276517.1. In one embodiment, an SP1 protein may comprise an amino acid sequence of one monomer as set forth in UniProt reference Q9AR79.

In one embodiment, the auxiliary protein is a DNA clamp. DNA clamps, also known as a sliding clamps or beta clamps or DnaN or Proliferating cell nuclear antigen (PCNA), are a class of proteins that enclose polynucleotides. DNA clamps are found in bacteria, archaea, eukaryotes and some viruses. DNA clamps are oligomeric toroidal proteins with a central channel of about 2-4 nm in diameter (similar for most orthologs), through which the polynucleotide passes. They are very well studied and the structures of many DNA clamps are known. Despite their name, DNA clamps are not necessarily specific to DNA. DNA clamps typically enclose dsDNA, but may also enclose ssDNA.

For example, the auxiliary protein may, in one embodiment, be a bacterial DNA clamp, or a modified verison thereof. The auxiliary protein may be a dimer, for example a homodimer, such as a homodimer composed of two identical beta subunits of a beta clamp, a specific example of which is DNA polymerase III beta clamp. An example of a bacterial DNS clamp amino acid sequence (from E. coli) is described in the UniProt database under reference P0A988. An example of a bacterial DNS clamp amino acid sequence (from E. coli) is described in the PDB under reference 1MMI. In one embodiment, a DNA clamp protein may comprise an amino acid sequence of one monomer as set forth in UniProt reference P0A988 or in the PDB under reference 1MMI.

In another embodiment, the auxiliary protein may be a DNA clamp of archaeal or eukaryotic origin, or a modified verison thereof. The auxiliary protein may, for example, be a trimer, for example a homotrimer, such as a trimer composed of three molecules of PCNA. An example of a eukaryotic (human) DNA clamp amino acid sequence is described in the UniProt database under reference P12004. An example of a human DNA clamp amino acid sequence is described in the PDB under reference laxc. In one embodiment, a DNA clamp protein may comprise an amino acid sequence of one monomer as set forth in UniProt reference P12004 or in the PDB under reference laxc. An example of an archaeal (P. furiosus) DNA clamp amino acid sequence is described in the UniProt database under reference O73947. An example of an archaeal (P. furiosus) DNA clamp amino acid sequence is described in the PDB under reference 1ISQ. In one embodiment, a DNA clamp protein may comprise an amino acid sequence of one monomer as set forth in UniProt reference O73947 or in the PDB under reference 1ISQ.

In another embodiment, the auxiliary protein may be a viral DNA clamp, such as a DNA clamp from T4 bacteriophage, or a modified verison thereof. For example, the auxiliary protein may be gp45. Gp45, for example, is a trimer similar in structure to PCNA but which lacks sequence homology to either PCNA or the bacterial beta clamp. An example of a viral (T4 bacteriophage) DNA clamp amino acid sequence is described in the UniProt database under reference P04525. An example of a viral (T4 bacteriophage) DNA clamp amino acid sequence is described in the PDB under reference 1CZD. In one embodiment, a DNA clamp protein may comprise an amino acid sequence of one monomer as set forth in UniProt reference P04525 or in the PDB under reference 1CZD.

In one embodiment, the auxiliary protein is a portal complex protein. A portal complex protein is a protein that in nature forms part of a specialised portal for entry of polynucleotides into and out of the viral capsid in any one of a large number of viruses, such as bacteriophages.

The portal complex protein can, for example be any one of a number of toroidal proteins that make up the bacteriophage. The toroidal (ring-like) proteins typically have a central channel. The toroidal protein typically has dimensions as defined herein for the auxiliary protein, either before or after modification. The toroidal protein typically has one or more properties, such as water solubility, one or more interfaces optimised for docking to another toroidal protein, robust stability under a wide range of extreme conditions.

Proteins that form the portal complexes are well known in the art, and structures are known for many of the proteins that make up the complexes. For example bacteriophages whose portal machinery is well characterised include: Phi29, T4, G20C, SPP1 and P22 bacteriophages. The portal complex protein in the pore complex is typically oligomeric (for example homooligomeric). For example, the portal complex protein may be formed from about 6 to more than about 14 monomeric subunits, such as about 12 subunits.

The portal complex protein may be the major protein in the multi-protein complex. This is usually called the “portal protein”. The portal protein is typically a dodecameric oligomer formed from 12 identical units, but may have a different number of oligomers, or be heterooligomeric. The structures are many portal proteins are known. The exact dimensions vary between each protein class and ortholog. Typically the minimum constriction in the central channel of the portal protein has a diameter in the range of about 1 nm to about 4 nm.

The portal protein may be adapted to span the membrane. A portal protein that are able to span the membrane may be used in the disclosed pore complexes as an auxiliary protein, and/or as a transmembrane pore. The portal protein in some embodiments may be one of the proteins shown in the Table below.

| | PDB entry | Uniprot entry |
|---|---|---|
| Protein | (rcsb.org/) | (uniprot.org/) |
| Phi29 portal protein: | 1FOU | P04332 |
| G20C | 4ZJN | A7XXR3 |
| T4 portal protein (gp20) | 3JA7 | P13334 |
| SPP1 portal protein (gp6) | 2JES | P54309 |
| P22 portal protein | 4V4K | P26744 |

In each organism the full portal complex will contain a number of separate toroidal oligomeric proteins, which are docked to the “portal protein” and to each other to create a continuous central channel through which polynucleotide can pass. The auxiliary protein may be, or comprise, any one or more of such “docked” or “accessory” proteins. The docked protein may, for example, be an “adapter protein”, a “stopper protein”, or a “motor protein” component of a portal complex. These are well characterised for the well known bacteriophages, many structures are known, and the dimensions of the inner channel through which the polynucleotide will pass typically vary from lnm to more than 4 nm.

Specific examples of toroidal proteins that can be used as the auxiliary protein include gp15 and gp16 from SPP1 bacteriophage, and other orthologs. Gp15, or the “adaptor protein”, docks to the bottom of the portal protein (gp6), and g16, or the “stopper protein”, docks to the bottom of Gp15.

The Gp15 and gp16 proteins contain inner channels with diameters of less than about 1 nm to greater than about 2 nm. Like the other auxiliary proteins disclosed herein, the inner channels of the Gp15 and gp16 proteins can be widened or narrowed to improve analyte discrimination or passage through mutagenesis (mutating residues in the constrictions, adding residues into loops, deleting loops, etc), directed by molecular structures and molecular modelling where required.

In one embodiment, the pore complex may comprise a portal protein as the transmembrane pore and a “docked” portal complex protein as the auxiliary protein. The pore complex may, for example, comprise two or more “docked” proteins.

| | PDB entry | Uniprot entry |
|---|---|---|
| Protein | (rcsb.org/) | (uniprot.org/) |
| Gp15 from SPP1 | 2KBZ | Q38584 |
| bacteriophage | | |
| Gp16 from SPP1 | 2KCA | O48446 |
| bacteriophage | | |

In one embodiment, the auxiliary protein is a motor protein. The motor protein is toroidal in structure, having a central channel for accommodating DNA or RNA in single-stranded or double-stranded form. The motor protein is oligomeric, typically being formed from about 6 or more monomeric subunits. The oligomer can be a homoligomer or a heteroligomer. They have a central channel for accommodating DNA or RNA in single-stranded or double-stranded form.

Some examples of motor proteins that function on single-stranded polynucleotides include, but not limited to: RepA (˜1.9 nm minimum diameter channel), TrwB (˜1.5 nm minimum diameter channel), ssoMCM (˜1.8 nm minimum diameter channel), Rho (˜1.7 nm minimum diameter channel), El helicase (˜1.3 nm minimum diameter channel), T7-gp4D (˜1.2 nm minimum diameter channel).

Some examples of motor proteins that function on double-stranded polynucleotides include, but not limited to: FtsK (˜3.4 nm minimum diameter channel), Phi29 gp10 (˜3.6 nm minimum diameter channel), P22 gpl (˜3.5 nm minimum diameter channel), T4 gp17 (˜3.6 nm minimum diameter channel), T7 gp8 (˜4.0 nm minimum diameter channel), HK97 family phage portal protein (˜3.3 nm minimum diameter channel).

In one embodiment, the auxiliary protein is another toroidal protein, For example, the toroidal protein may, in one embodiment, be Lambda exonuclease. Lambda exonuclease is a well characterised homotrimeric toroidal protein, with an inner channel with a diameter of about 1.5 nm to 3 nm. (PDB 1AVQ, Uniprot P03697). In one embodiment, a DNA clamp protein may comprise an amino acid sequence of one monomer as set forth in UniProt reference P03697 or in the PDB under reference 1AVQ.

Another example of the toroidal protein is TRAP. TRAP is a bacterial RNA-binding protein from organisms such as Bacillus subtilis and Bacillus Stearothermophilus. TRAP has 11 subunits arranged in a ring-like structure, with a central channel with diameter of about 2 nm (PDB 1QAW, uniprot Q9X6J6). In one embodiment, a DNA clamp protein may comprise an amino acid sequence of one monomer as set forth in UniProt reference Q9X6J6 or in the PDB under reference 1QAW.

In one embodiment, the auxiliary protein is not a polynucleotide binding protein. In one embodiment, the auxiliary protein is not a functional polynucleotide binding protein, e.g. the auxiliary protein is not a polynucleotide binding protein having enzymatic activity. The auxiliary protein may be a protein other than a nucleic acid handling enzyme, for example, the auxiliary protein is not a helicase or a polymerase, or a protein derived from such an enzyme. In one embodiment, the auxiliary protein has no enzymatic activity. In one embodiment, the auxiliary protein does not undergo a conformational change upon passage of the target polynucleotide through the continuous channel formed in the pore complex.

In one embodiment, the auxiliary protein or peptide is a component of a nanopore system, or a modified component of such a system, other than a component that forms a transmembrane pore. An example of such a component is CsgF, or a truncated version of CsgF. In one embodiment, the pore complex comprises a CsgF protein or peptide and a CsgG pore, or a homologue or modified version, such as a fragment, thereof. In another embodiment, the pore complex comprises a CsgF protein or peptide and a non-CsgG pore, homologue or modified version, such as a fragment, thereof.

The auxiliary protein is, in one embodiment, a transmembrane protein pore. The auxiliary protein and the nanopore may, where the auxiliary protein is a transmembrane protein pore, be the same or different. A pore complex comprising an auxiliary protein which is a nanopore may be referred to as a double pore. The nanopore and the auxiliary protein may be referred to in this embodiment as the first and second pores. The auxiliary protein may be any of the transmembrane protein pores defined herein.

In one embodiment, the auxiliary peptide is a CsgF peptide, which can be a truncated, mutant and/or variant CsgF peptide. In one embodiment, where the nanopore is a CsgG pore, the auxiliary peptide is not a CsgF peptide and the auxiliary protein is not CsgF. In one embodiment, where the auxiliary peptide is a CsgF peptide, the nanopore is not a CsgG pore, or a homologue or mutant thereof. In another embodiment, the pore complex has more than two constriction sites or reader heads, wherein at least one is a constriction of the CsgG pore, one is introduced by the CsgF peptide, and a further constriction site is introduced by a second auxiliary protein or peptide present in the pore complex.

In one embodiment, the modified CsgF peptide is a peptide wherein said modification in particular refers to a truncated CsgF protein or fragment, comprising an N-terminal CsgF peptide fragment defined by the limitation to contain the constriction region and to bind CsgG monomers, or homologues or mutants thereof. Said modified CsgF peptide may additionally comprise mutations or homologous sequences, which may facilitate certain properties of the pore complex. In a particular embodiment, modified CsgF peptides comprise CsgF protein truncations as compared to the wild-type preprotein (SEQ ID NO:5) or mature protein (SEQ ID NO:6) sequence, or homologues thereof. These modified peptides are intended to function as a pore complex component introducing an additional constriction site or reader head, within the CsgG-like pore formed by CsgG and the modified or truncated CsgF peptide.

The truncated CsgF peptide lacks: the C-terminal head; the C-terminal head and a part of the neck domain of CsgF; or the C-terminal head and neck domains of CsgF. The CsgF peptide may lack part of the CsgF neck domain, e.g. the CsgF peptide may comprise a portion of the neck domain, such as for example, from amino acid residue 36 at the N-terminal end of the neck domain (see SEQ ID:NO:6) (e.g. residues 36-40, 36-41, 36-42, 36-43, 36-45,36-46 up to residues 36-50 or 36-60 of SEQ ID NO: 6). The CsgF peptide preferably comprises a CsgG-binding region and a region that forms a constriction in the pore. The CsgG-binding region typically comprises residues 1 to 8 and/or 29 to 32 of the CsgF protein (SEQ ID NO: 6 or a homologue from another species) and may include one or more modifications. The region that forms a constriction in the pore typically comprises residues 9 to 28 of the CsgF protein (SEQ ID NO: 6 or a homologue from another species) and may include one or more modifications. Residues 9 to 17 comprise the conserved motif N₉PXFGGXXX₁₇ and form a turn region. Residues 9 to 28 form an alpha-helix. X₁₇ (N17 in SEQ ID NO: 6) forms the apex of the constriction region, corresponding to the narrowest part of the CsgF constriction in the pore. The CsgF constriction region also makes stabilising contacts with the CsgG beta-barrel, primarily at residues 9, 11, 12, 18, 21 and 22 of SEQ ID NO: 6.

The CsgF peptide typically has a length of from 28 to 50 amino acids, such as 29 to 49, 30 to 45 or 32 to 40 amino acids. Preferably the CsgF peptide comprises from 29 to 35 amino acids, or 29 to 45 amino acids. The CsgF peptide comprises all or part of the FCP, which corresponds to residues 1 to 35 of SEQ ID NO: 6. Where the CsgF peptide is shorter that the FCP, the truncation is preferably made at the C-terminal end.

The CsgF fragment of SEQ ID NO:6 or of a homologue or mutant thereof may have a length of 24, 25, 26, 27, 28, 29, 30, 31,32, 33, 34, 35, 36, 37, 38, 39, 40, 41, 42, 43, 44, 45, 46, 47, 48, 49, 50, 51, 52, 53, 54 or 55 amino acids.

The CsgF peptide may comprise the amino acid sequence of SEQ ID NO: 6 from residue 1 up to any one of residues 25 to 60, such as 27 to 50, for example, 28 to 45 of SEQ ID NO: 6, or the corresponding residues from a homologue of SEQ ID NO: 6, or variant of either thereof.

More specifically, the CsgF peptide may comprise residues 1 to 29 of SEQ ID NO: 6, or a homologue or variant thereof.

Examples of such CsgF peptides comprises, consist essentially of or consist of residues 1 to 34 of SEQ ID NO: 6, residues 1 to 30 of SEQ ID NO: 6, residues 1 to 45 of SEQ ID NO: 6, or residues 1 to 35 of SEQ ID NO: 6, and homologues or variants of any thereof. In the CsgF peptide, one or more residues may be modified. For example, the CsgF peptide may comprise a modification at a position corresponding to one or more of the following positions in SEQ ID NO: 6: G1, T4, F5, R8, N9, N11, F12, A26 and Q29, such as the introduction of a cysteine, a hydrophobic amino acid, a charged amino acid, a non-native reactive amino acid, or photoreactive amino acid at any one or more of these positions.

For example, the CsgF peptide may comprise a modification at a position corresponding to one or more of the following positions in SEQ ID NO: 6: N15, N17, A20, N24 and A28. The CsgF peptide may comprise a modification at a position corresponding to D34 to stabilise the CsgG-CsgF complex. In particular embodiments, the CsgF peptide comprises one or more of the substitutions: N15S/A/T/Q/G/L/V/I/F/Y/W/R/K/D/C, N17S/A/T/Q/G/L/V/I/F/Y/W/R/K/D/C, A20S/T/Q/N/G/L/V/I/F/Y/W/R/K/D/C, N24S/T/Q/A/G/L/V/I/F/Y/W/R/K/D/C, A28S/T/Q/N/G/L/V/I/F/Y/W/R/K/D/C and D34F/Y/W/R/K/N/Q/C. The CsgF peptide may, for example, comprise one or more of the following substitutions: G1C, T4C, N17S, and D34Y or D34N.

### Nanopore

A nanopore is a hole or channel through a membrane that permits hydrated ions driven by an applied potential to flow across or within the membrane. The nanopore in the pore complex may be a protein pore that crosses the membrane to some degree, or may be a non-protein pore that has a structure that crosses the membrane to some degree, such as a polynucleotide pore or solid state pore. The pore may be a DNA origami pore. The pore may be biological or artificial.

The nanopore is, in one embodiment, a transmembrane protein pore. The transmembrane protein pore typically spans the entire membrane and may have a structure that extends beyond the membrane on one or both sides. A transmembrane protein pore is a single or multimeric protein that permits hydrated ions to flow from one side of a membrane to the other side of the membrane. The transmembrane protein pore comprises a channel that allows a polynucleotide, such as DNA or RNA, to move, or be moved, into and/or through the pore.

The transmembrane protein pore may be a monomer or an oligomer. The oligomer is preferably made up of several repeating subunits, such as at least 6, at least 7, at least 8, at least 9, at least 10, at least 11, at least 12, at least 13, at least 14, at least 15, or at least 16 subunits. For example, the pore may be a hexameric, heptameric, octameric or nonameric pore. The pore may be a homo-oligomer in which all of the subunits are identical, or a hetero-oligomer comprising two or more, such as 3, 4, 5 or 6, different subunits.

The transmembrane protein pore typically comprises a barrel or channel through which the ions may flow. The subunits of the pore typically surround a central axis and contribute strands to a transmembrane β-barrel or channel or a transmembrane α-helix bundle or channel.

The barrel or channel of the transmembrane protein pore typically comprises amino acids that facilitate interaction with polynucleotides. These amino acids are preferably located near a constriction (such as within 1, 2, 3, 4 or 5 nm) of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin (αHL), anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and α outer membrane proteins, such as WZA.

The transmembrane pore may be derived from or based on Msp, α-hemolysin (α-HL), lysenin, CsgG, SP1, hemolytic protein fragaceatoxin C (FraC), a secretin such as InvG or GspD, leukocidin, aerolysin, NetB, a porin such as OmpG (outer membrane protein G) or VdaC (voltage dependent anion channel), VCC (vibrio cholerae cytolysin), anthrax protective antigen, or an ATPase rotor such as C10 Rotor ring of the Yeast Mitochondrial ATPase, K ring of V-ATPase from Enterococcus hirae, C11 Rotor ring of the Ilycobacter tartaricus ATPase, or C13 Rotor ring of the Bacillus pseudofirmus ATPase. Thus, in some embodiments, the transmembrane protein nanopore is selected from MspA, α-hemolysin, CsgG, lysenin, InvG, GspD, leukocidin, FraC, aerolysin, NetB, and functional homologues and fragments thereof. Structures for the transmembrane protein pores are available in protein data banks, for example MspA, α-HL and CsgG are protein data bank entries 1UUN, 7AHL and 4UV3, respectively.

In one embodiment, the nanopore is a CsgG pore, such as for example CsgG from E. coli Str. K-12 substr. MC4100, or a homologue or mutant thereof. Mutant CsgG pores may comprise one or more mutant monomers. The CsgG pore may be a homopolymer comprising identical monomers, or a heteropolymer comprising two or more different monomers. Suitable pores derived from CsgG are disclosed in WO 2016/034591, WO2017/149316, WO2017/149317, WO2017/149318 and International patent application nos. PCT/GB2018/051191 and PCT/GB2018/051858.

The transmembrane pore may be derived from lysenin. Suitable pores derived from lysenin are disclosed in WO 2013/153359.

In one embodiment, the nanopore is a secretin pore, such as for example GspD or InvG, or a homologue or mutant thereof. Secretin nanopores are described in WO2018/146491.

In one embodiment, the transmembrane pore may be a portal protein, or a modified portal protein. In this embodiment, it is preferred that the portal protein, which is the transmembrane pore is complexed with an auxiliary protein that is a portal protein accessory protein. The first constriction, or reader hesd, is formed by the portal protein and the second constriction, or reader head, is formed by the accessory protein. The portal protein used as transmembrane pore may be modified such that it is able to span the membrane. In one embodiment, the complex comprising a portal protein as the transmembrane pore is not a naturally occurring complex. The non-naturally occurring portal complex may comprise one or more modified protein and/or may lack one or more component of the naturally occurring pore complex.

Proteins that form the portal complexes are well known in the art, and structures are known for many of the proteins that make up the complexes. For example bacteriophages whose portal machinery is well characterised include: Phi29, T4, G20C, SPP1 and P22 bacteriophages as described above. The portal complex protein in the pore complex is typically oligomeric (for example homooligomeric). For example, the portal complex protein may be formed from about 6 to more than about 14 monomeric subunits, such as about 12 subunits.

The portal protein is typically a dodecameric oligomer formed from 12 identical units, but may have a different number of oligomers, such as from 6, 7, 8, 9 or 10 to 11, 12, 13 or 14 subunits, and/or be heterooligomeric. The structures are many portal proteins are known. The exact dimensions vary between each protein class and ortholog. Typically the minimum constriction in the central channel of the portal protein has a diameter in the range of about 1 nm to about 4 nm. The inner channel of the portal protein can be widened or narrowed to improve analyte discrimination or passage of polynucleotides through the pore, for example by mutagenesis (mutating residues in the constrictions, adding residues into loops, deleting loops, etc), directed by molecular structures and molecular modelling where required.

In some embodiments, the transmembrane nanopore is a naturally occurring transmembrane nanopore, or a pore derived from a naturally occurring transmembrane nanopore, such as a modified version thereof. In some embodiments, the transmembrane protein nanopore within the pore complex is not a wild-type pore, but comprises mutations or modifications to increase its nucleotide sensing properties. For example, mutations that alter the number, size, shape, placement or orientation of the constriction within the channel may be made to the transmembrane protein nanopore. The pore complex comprising a modified transmembrane protein nanopore may be prepared by known genetic engineering techniques that result in the insertion, substitution and/or deletion of specific targeted amino acid residues in the polypeptide sequence.

In the case of an oligomeric transmembrane protein pore, the mutations may be made in each monomeric polypeptide subunit, or any one or more of the monomers. Suitably, in one embodiment of the invention the mutations described are made to all monomers within the oligomeric protein. A mutant monomer is a monomer whose sequence varies from that of a wild-type pore monomer and which retains the ability to form a pore. Methods for confirming the ability of mutant monomers to form pores are well-known in the art.

In one embodiment, the nanopore is a solid-state nanopore. A solid-state nanopore is typically a nanometer-sized hole formed in a synthetic membrane (usually SiNx or SiO₂). The pore is usually fabricated by focused ion or electron beams, so the size of the pore can be tuned freely. The solid-state nanopore may be made in, for example a silicon nitride or graphene membrane, or a membrane made of a modifed version of these solid-state materials.

### Stabilisation of Pore Complex

The pore may be stabilised by covalent attachment of the auxiliary protein or peptide to the nanopore. The covalent linkage may for example be a disulphide bond, or click chemistry. By way of further example cysteine residues may be connected by means of a linker such as BMOE. The auxiliary protein or peptide and/or the transmembrane protein nanopore may be modified to facilitate such covalent interactions.

In the pore complex, the nanopore, which is preferably a transmembrane protein nanopore, may be attached to the auxiliary protein by hydrophobic interactions and/or by one or more disulphide bond. One or more, such as 2, 3, 4, 5, 6, 8, 9, for example all, of the monomers in either one or both pores may be modified to enhance such interactions. This may be achieved in any suitable way. Further suitable interactions include salt bridges, electrostatic interactions, and Pi-Pi interactions.

At least one cysteine residue in the amino acid sequence of the transmembrane protein nanopore at the interface between the nanopore and auxiliary protein may be disulphide bonded to at least one cysteine residue in the amino acid sequence of the auxiliary protein at the interface between the nanopore and auxiliary protein . The cysteine residue in the nanopore and/or the cysteine residue in the auxiliary protein may be a cysteine residue that is not present in the wild type transmembrane protein pore monomer or in the wild-type auxiliary protein. Multiple disulphide bonds, such as from 2, 3, 4 , 5, 6, 7, 8 or 9 to 16, 18, 24, 27, 32, 36, 40, 45, 48, 54, 56 or 63, may form between the nanopore and auxiliary protein in the pore complex. One or both of the nanopore and the auxiliary protein may comprise at least one monomer, or subunit, such as up to 8, 9 or 10 monomers or subunits, that comprises a cysteine residue at the interface between the nanopore and auxiliary protein. For example, in CsgG, the cysteine residue may be included at a position corresponding to R97, I107, R110, Q100, E101, N102 and/or L113 of SEQ ID NO: 3.

The nanopore and/or auxiliary protein may comprise one or more hydrophobic amino acid residue at the interface between the nanopore and auxiliary protein, which is more hydrophobic than the residue present at the corresponding position in the wild type nanopore or auxiliary protein. At least one monomer, or subunit, in the nanopore and/or at least one monomer, or subunit, in the auxiliary protein may comprise at least one residue at the interface between the nanopore and auxiliary protein, which residue is more hydrophobic than the residue present at the corresponding position in the wild type pore or auxiliary protein monomer. For example, from 2 to 10, such as 3, 4, 5, 6, 7, 8 or 9, residues in the nanopore and/or the auxiliary protein may be more hydrophobic that the residues at the same positions in the corresponding wild type nanopore and/or the auxiliary protein. Such hydrophobic residues strengthen the interaction between the nanopore and the auxiliary protein in the pore complex. Where the residue at the interface in the wild type nanopore or auxiliary protein is R, Q, N or E, the hydrophobic residue is typically I, L, V, M, F, W or Y. Where the residue at the interface in the wild type nanopore or auxiliary protein is I, the hydrophobic residue is typically L, V, M, F, W or Y. Where the residue at the interface in the wild type nanopore or auxiliary protein is L, the hydrophobic residue is typically I, V, M, F, W or Y. For example, where the nanopore and/or auxiliary protein in the complex is CsgG, the at least one residue at the interface between the nanopore and auxiliary protein may be at a position corresponding to R97, I107, R110, Q100, E101, N102 and or L113 of SEQ ID NO: 3.

The nanopore and/or auxiliary protein in the pore complex may comprise one or more monomer that comprises one or more cysteine residue at the interface between the pores and one or more monomer that comprises one or more introduced hydrophobic residue at the interface between the pores, or may comprise one or more monomer that comprises such cysteine residues and such hydrophobic residues. For example, one or more, such as any 2, 3, or 4, of the positions in the monomer at the interface (where the pore is CsgG, these can correspond to the positions at R97, I107, R110, Q100, E101, N102 and or L113 of SEQ ID NO: 3) may comprise a cysteine (C) residue and one or more, such as any 2, 3 or 4, of the positions in the monomer (where the pore is CsgG, these can correspond to the positions at R97, I107, R110, Q100, E101, N102 and or L113 of SEQ ID NO: 3) may comprise a hydrophobic residue, such as I, L, V, M, F, W or Y.

Molecular dynamics simulations can be performed to establish which residues in the auxiliary protein and nanopore come into close proximity. This information can be used to design auxiliary protein and/or transmembrane protein nanopore mutants that could increase the stability of the complex. For example, simulations can be performed using the GROMACS package version 4.6.5, with the GROMOS 53a6 force field and the SPC water model using cryo-EM structure of the proteins. The complex can be solvated and then energy minimised using the steepest descents algorithm. Throughout the simulation, restraints can be applied to the backbones of the proteins, however, the residue side chains can be free to move. The system can be simulated in the NPT ensemble for 20 ns, using the Berendsen thermostat and Berendsen barostat to 300 K. Contacts between the auxiliary protein and nanopore can be analysed using GROMACS analysis software and/or locally written code. Two residues can be defined as having made a contact if they come within 3 Angstroms of each other.

For example, in a pore complex, the interaction between a CsgF peptide and a CsgG pore may, for example, be stabilised by hydrophobic interactions or electrostatic interactions at a position corresponding to one or more of the following pairs of positions of SEQ ID NO: 6 and SEQ ID NO: 3, respectively: 1 and 153, 4 and 133, 5 and 136, 8 and 187, 8 and 203, 9 and 203, 11 and 142, 11 and 201, 12 and 149, 12 and 203, 26 and 191, and 29 and 144. The residues in CsgF and/or CsgG at one or more of these positions may be modified in order to enhance the interaction between CsgG and CsgF in the pore.

The covalent link or binding is, for example, via cysteine linkage, wherein the sulfhydryl side group of cysteine covalently links with another amino acid residue or moiety and/or via an interaction between non-native (photo)reactive amino acids. (Photo-)reactive amino acids are referring to artificial analogs of natural amino acids that can be used for crosslinking of protein complexes, and may be incorporated into proteins and peptides in vivo or in vitro. Photo-reactive amino acid analogs in common use are photoreactive diazirine analogs to leucine and methionine, and para-benzoyl-phenyl-alanine, as well as azidohomoalanine, homopropargylglycyine, homoallelglycine, p-acetyl-Phe, p-azido-Phe, p-propargyloxy-Phe and p-benzoyl-Phe (Wang et al. 2012; Chin et al. 2002). Upon exposure to ultraviolet light, they are activated and covalently bind to interacting proteins that are within a few angstroms of the photo-reactive amino acid analog.

The pore complex can be made and disulphide bond formation can be induced by using oxidising agents (eg: Copper-orthophenanthroline). Other interactions (eg: hydrophobic interactions, charge-charge interactions/electrostatic interactions) can also be used in those positions instead of cysteine interactions. In another embodiment, unnatural amino acids can also be incorporated in those positions. In this embodiment, covalent bonds made be made by via click chemistry. For example, unnatural amino acids with azide or alkyne or with a dibenzocyclooctyne (DBCO) group and/or a bicyclo[6.1.0]nonyne (BCN) group may be introduced at one or more of these positions.

For example, the CsgG pore may comprise at least one, such as 2, 3, 4, 5, 6, 7, 8, 9 or 10, CsgG monomers that is/are modified to facilitate attachment to the CsgF peptide, or other auxiliary protein or peptide. For example a cysteine residue may be introduced at one or more of the positions corresponding to positions 132, 133, 136, 138, 140, 142, 144, 145, 147, 149, 151, 153, 155, 183, 185, 187, 189, 191, 201, 203, 205, 207 and 209 of SEQ ID NO: 3, and/or at any one of the positions identified in Table 4 as being predicted to make contact with CsgF, to facilitate covalent attachment to CsgF, or another auxiliary protein. As an alternative or addition to covalent attachment via cysteine residues, the pore may be stabilised by hydrophobic interactions or electrostatic interactions. To facilitate such interactions, a non-native reactive or photoreactive amino acid at a position corresponding to one or more of positions 132, 133, 136, 138, 140, 142, 144, 145, 147, 149, 151, 153, 155, 183, 185, 187, 189, 191, 201, 203, 205, 207 and 209 of SEQ ID NO: 3.

For example, the CsgF peptide may be modified to facilitate attachment to the CsgG pore. For example a cysteine residue may be introduced at one or more of the positions corresponding to positions 1, 4, 5, 8, 9, 11, 12, 26 or 29 of SEQ ID NO: 6, and/or at any one of the positions identified in Table 4 as being predicted to make contact with CsgF, to facilitate covalent attachment to CsgG. As an alternative or addition to covalent attachment via cysteine residues, the pore may be stabilised by hydrophobic interactions or electrostatic interactions. To facilitate such interactions, a non-native reactive or photoreactive amino acid at a position corresponding to one or more of positions 1, 4, 5, 8, 9, 11, 12, 26 or 29 of SEQ ID NO: 6.

Such stabilising mutations can be combined with any other modifications to the auxiliary protein and/or transmembrane protein nanopore, for example the modifications to improve the interaction of the pore complex with a polynucleotide, or to improve the properties of the reader head in the nanopore or auxiliary protein.

In one embodiment, the nanopore may be isolated, substantially isolated, purified or substantially purified. A pore is isolated or purified if it is completely free of any other components, such as lipids or other pores. A pore is substantially isolated if it is mixed with carriers or diluents which will not interfere with its intended use. For instance, a pore is substantially isolated or substantially purified if it is present in a form that comprises less than 10%, less than 5%, less than 2% or less than 1% of other components, such as triblock copolymers, lipids or other pores. Alternatively, the pore may be present in a membrane. Suitable membranes are discussed below.

The pore complex of may be present in a membrane as an individual or single pore. Alternatively, the pore complex may be present in a homologous or heterologous population of two or more pores.

The auxiliary protein may be attached directly to the transmembrane protein nanopore, or the two proteins may be attached using a linker, such as a chemical crosslinker or a peptide linker.

Suitable chemical crosslinkers are well-known in the art. Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate. The most preferred crosslinker is succinimidyl 3-(2-pyridyldithio)propionate (SPDP). Typically, the molecule is covalently attached to the bifunctional crosslinker before the molecule/crosslinker complex is covalently attached to the mutant monomer but it is also possible to covalently attach the bifunctional crosslinker to the monomer before the bifunctional crosslinker/monomer complex is attached to the molecule.

The linker is preferably resistant to dithiothreitol (DTT). Suitable linkers include, but are not limited to, iodoacetamide-based and Maleimide-based linkers.

The auxiliary protein may be genetically fused to the transmembrane protein nanopore. For example, in an embodiment where the ring shaped auxiliary protein has the same symmetry as the nanopore, each monomer, or subunit, of the nanopore may be fused to a monomer, or subunit, of the auxiliary protein. The monomer and protein are genetically fused if the whole construct is expressed from a single polynucleotide coding sequence. The monomer, or subunit, auxiliary protein may be directly fused to a monomer, or subunit, of the transmembrane protein nanopore. Alternatively, the monomer, or subunit, auxiliary protein may be fused to a monomer, or subunit, of the transmembrane protein nanopore via one or more linkers.

In one embodiment, the hybridization linkers described in as WO 2010/086602 may be used. Alternatively, peptide linkers may be used. The length, flexibility and hydrophilicity of the peptide linker are typically designed such that it does not to disturb the functions of the monomer and molecule. In one embodiment, the peptide linker is typically of between 1 and 20, preferably 2 and 10, such as 3 and 5, for example 4, amino acids in length. The linkers may, for example, be composed of one or more of the following amino acids: lysine, serine, arginine, proline, glycine and alanine. Examples of suitable flexible peptide linkers are stretches of 2 to 20, such as 4, 6, 8, 10 or 16, serine and/or glycine amino acids. Examples of rigid linkers are stretches of 2 to 30, such as 4, 6, 8, 16 or 24, proline amino acids.Examples of suitable linkers include, but are not limited to, the following: GGGS, PGGS, PGGG, RPPPPP, RPPPP, VGG, RPPG, PPPP, RPPG, PPPPPPPPP, PPPPPPPPPPPP, RPPG, GG, GGG, SG, SGSG, SGSGSG, SGSGSGSG, SGSGSGSGSG and SGSGSGSGSGSGSGSG wherein G is glycine, P is proline, R is arginine, S is serine and V is valine.

Appropriate linking groups may be designed using conventional modelling techniques. The linker is typically sufficiently flexible to allow the monomers, or subunits, to assemble into their respective protein oligomers, and to align along their common symmetry axis in order to produce a continuous channel within the pore complex.

### Closing Gaps Between the Nanopore and Auxiliary Protein.

The auxiliary protein and/or transmembrane protein nanopore may contain bulky residues at one or more, such as 2, 3, 4, 5, 6 or 7, positions at the interface between the proteins in the pore complex, particularly in an embodiment where in the pore complex the auxiliary protein is located outside the channel of the transmembrane protein pore. The auxiliary protein and/or transmembrane protein nanopore may be modified to comprise amino acids that are bulkier than the residues present at the corresponding positions in the wild type proteins. The bulk of these residues prevents holes from forming in the walls of the pore at the interface between the proteins in the pore complex. Where the residue at the interface is A, the bulky residue is typically I, L, V, M, F, W, Y, N, Q, S or T. Where the residue present at the interface in the wild type protein is T, the bulky residue is typically L, M, F, W, Y, N, Q, R, D or E. Where the residue present at the interface in the wild type protein is V, the bulky residue is typically I, L, M, F, W, Y, N, Q. Where the residue present at the interface in the wild type protein is L, the bulky residue is typically M, F, W, Y, N, Q, R, D or E. Where the residue present at the interface in the wild type protein is Q, the bulky residue is typically F, W or Y. Where the residue present at the interface in the wild type protein is S, the bulky residue is typically M, F, W, Y, N, Q, E or R. For example, where the pore is CsgG, the at least one bulky residue at the interface between the first and second pores is typically at a position corresponding to A98, A99, T104, V105, L113, Q114 or S115 of SEQ ID NO: 3. Gaps can also be filled by creating energetic barriers for the flow of ions. For example, electrostatic charges can be introduced by mutation to create electrostatic barriers to cations and/or anions.

Molecular modelling can be performed to establish where gaps at the interface between the auxiliary protein and nanopore exist at the interface between the two proteins. This information can be used to design auxiliary protein and/or transmembrane protein nanopore mutants that fit together more precisely, and hence to reduce any current leakage that occurs when the pore complex is present in a membrane and an ionic current flows through the pore complex. For example, simulations can be performed using the GROMACS package version 4.6.5, with the GROMOS 53a6 force field and the SPC water model using cryo-EM structure of the proteins. The complex can be solvated and then energy minimised using the steepest descents algorithm. Throughout the simulation, restraints can be applied to the backbones of the proteins, however, the residue side chains can be free to move. The system can be simulated in the NPT ensemble for 20 ns, using the Berendsen thermostat and Berendsen barostat to 300 K. Gaps between the auxiliary protein and nanopore can be analysed using GROMACS analysis software and/or locally written code.

### Modifications to Improve Polynucleotide Sensing

The auxiliary protein, and/or the nanopore, may be modified to comprise one or more amino acid residues in its central channel region that reduce the negative charge compared to the charge in the central channel region of the wild type protein(s). At least one monomer in the auxiliary protein and/or at least one monomer in the nanopore may comprise at least one residue in the continuous channel, which residue has less negative charge than the residue present at the corresponding position in the wild type protein. The charge inside the channel is sufficiently neutral or positive such that negatively charged analytes, such as polynucleotides, are not repelled from entering the pore by electrostatic charges. Such charge altering mutations are known in the art.

For example, where the pore is CsgG at least one residue, such as 2, 3, 4 or 5 residues, in the channel region of the pore at a position corresponding to D149, E185, D195, E210 and/or E203 of SEQ ID NO: 3 may be a neutral or positively charged amino acid. At least one residue, such as 2, 3, 4 or 5 residues, in the channel region of the pore at a position corresponding to D149, E185, D195, E210 and/or E203 of SEQ ID NO: 3 is preferably N, Q, R or K.

The transmembrane protein pore and/or the auxiliary protein may comprise at least one residue in the constriction, which residue decreases, maintains or increases the length of the constriction compared to the wild type protein.

For example, in the CsgG pore, the length of the constriction may be increased by inserting residues into the region corresponding to the region between positions K49 and F56 of SEQ ID NO: 3. From 1 to 5, such as 2, 3, or 4 amino acid residues may be inserted at any one or more of the following positions defined by reference to SEQ ID NO: 3: K49 and P50, P50 and Y51, Y51 and P52, P52 and A53, A53 and S54, S54 and N55 and/or N55 and F56. Preferably from 1 to 10, such as 2 to 8, or 3 to 5 amino acid residues in total are inserted into the sequence of a monomer. Preferably, all of the monomers in the first pore and/or all of the monomers in the second pore have the same number of insertions in this region. The inserted residues may increase the length of the loop between the residues corresponding to Y51 and N55 of SEQ ID NO: 3. The inserted residues may be any combination of A, S, G or T to maintain flexibility; P to add a kink to the loop; and/or S, T, N, Q, M, F, W, Y, V and/or Ito contribute to the signal produced when an analyte interacts with the channel of the pore under an applied potential difference. The inserted amino acids may be any combination of S, G, SG, SGG, SGS, GS, GSS and/or GSG.

In the pore complex, the constriction nanopore and/or the constriction in the auxiliary protein may comprise at least one residue, such as 2, 3, 4 or 5 residues, which influences the properties of the pore complex when used to detect or characterise an analyte compared to when a pore complex with the corresponding wild-type constriction is used. For example, where the nanopore and/or auxiliary protein is CsgG, the at least one residue in the constriction of the barrel region of the pore may be at a position corresponding to Y51, N55, Y51, P52 and/or A53 of SEQ ID NO: 3. For example, the at least one residue may be Q or V at a position corresponding to F56 of SEQ ID NO: 3; A or Q at a position corresponding to Y51 of SEQ ID NO: 3; and/or V at a position corresponding to N55 of SEQ ID NO: 3.

In certain embodiments, where the nanopore and/or auxiliary protein is CsgG, the CsgG monomers in the pore complex may comprise a cysteine residue at a position corresponding to R97, I107, R110, Q100, E101, N102 and or L113 of SEQ ID NO: 3. A CsgG monomer may comprise a residue at a position corresponding to any one or more of R97, Q100, I107, R110, E101, N102 and L113 of SEQ ID NO: 3, which residue is more hydrophobic than the residue present at the corresponding position of SEQ ID NO: 3, wherein the residue at the position corresponding to R97 and/or 1107 is M, the residue at the position corresponding to R110 is I, L,

V, M, W or Y, and/or the residue at the position corresponding to E101 or N102 is V or M. The residue at a position corresponding to Q100 is typically I, L, V, M, F, W or Y; and or the residue at a position corresponding to L113 is typically I, V, M, F, W or Y.

In certain embodiments, where the nanopore and/or auxiliary protein is CsgG, the CsgG monomer in the nanopore and/or auxiliary protein may comprise a residue at a position corresponding to any one or more of A98, A99, T104, V105, L113, Q114 and S115 of SEQ ID NO: 3 which is bulkier than the residue present at the corresponding position of SEQ ID NO: 3, such as the corresponding position of any one of SEQ ID NOs: 68 to 88, wherein the residue at the position corresponding to T104 is L, M, F, W, Y, N, Q, D or E, the residue at the position corresponding to L113 is M, F, W, Y, N, G, D or E and/or the residue at the position corresponding to S115 is M, F, W, Y, N, Q or E. The residue at a position corresponding to A98 or A99, is typically I, L, V, M, F, W, Y, N, Q, S or T. The residue at a position corresponding to V105 is I, L, M, F, W, Y, N or Q. The residue at a position corresponding to Q114 is F, W or Y. The residue at a position corresponding to E210 is N, Q, R or K.

In certain embodiments, where the nanopore and/or auxiliary protein is CsgG, the CsgG monomer in the nanopore and/or auxiliary protein may comprise a residue in the barrel region of the pore at a position corresponding to any one or more of D149, E185, D195, E210 and E203 less negative charge than the residue present at the corresponding position of SEQ ID NO: 3, such as the corresponding position of any one of SEQ ID NOs: 68 to 88, wherein the residue at the position corresponding to D149, E185, D195 and/or E203 is K.

In certain embodiments, where the nanopore and/or auxiliary protein is CsgG, the CsgG monomer in the nanopore and/or auxiliary protein may comprise at least one residue in the constriction of the barrel region of the pore, which residue increases the length of the constriction compared to the wild type CsgG pore. The at least one residue is additional to the residues present in the constriction of the wild type CsgG pore. The length of the pore may, for example, be increased by inserting residues into the region corresponding to the region between positions K49 and F56 of SEQ ID NO: 3. From 1 to 5, such as 2, 3, or 4 amino acid residues may be inserted at any one or more of the following positions defined by reference to SEQ ID NO: 3: K49 and P50, P50 and Y51, Y51 and P52, P52 and A53, A53 and S54, S54 and N55 and/or N55 and F56. Preferably from 1 to 10, such as 2 to 8, or 3 to 5 amino acid residues in total are inserted into the sequence of the monomer. The inserted residues may increase the length of the loop between the residues corresponding to Y51 and N55 of SEQ ID NO: 3. The inserted residues may be any combination of A, S, G or T to maintain flexibility; P to add a kink to the loop; and/or S, T, N, Q, M, F, W, Y, V and/or Ito contribute to the signal produced when an analyte interacts with the barrel of the pore under an applied potential difference. The inserted amino acids may be any combination of S, G, SG, SGG, SGS, GS, GSS and/or GSG.

In certain embodiments, where the nanopore and/or auxiliary protein is CsgG, the CsgG monomer in the nanopore and/or auxiliary protein may comprise at least one residue in the constriction of the barrel region of the pore at a position corresponding to N55, P52 and/or A53 of SEQ ID NO: 3 that is different from the residue present in the corresponding wild type monomer, wherein the residue at a position corresponding to N55 is V.

Any two or more of the above described modifications may be present in the auxiliary protein or nanopore. In particular the monomer may comprise at least one said cysteine residue, at least one said hydrophobic residue, at least one said bulky residue, at least one said neutral or positively charged residue and/or at least one said residue that increases the length of the constriction.

In certain embodiments, where the nanopore and/or auxiliary protein is CsgG, the CsgG monomer in the nanopore and/or auxiliary protein may additionally comprise one or more, such as 2, 3, 4 or 5 residues, which influence the properties of the pore when used to detect or characterise an analyte compared to when a CsgG nanopore and/or CsgG auxiliary protein with a wild-type constriction is used, wherein the at least one residue in the constriction of the barrel region of the pore is at a position corresponding to Y51, N55, Y51, P52 and/or A53 of SEQ ID NO: 3. The at least one residue may be Q or V at a position corresponding to F56 of SEQ ID NO: 3; A or Q at a position corresponding to Y51 of SEQ ID NO: 3; and/or V at a position corresponding to N55 of SEQ ID NO: 3.

In some embodiments, the pore complex has improved polynucleotide reading properties when said complex is used in nucleotide sequencing i.e. display improved polynucleotide capture and/or nucleotide discrimination.

In particular, pore complexes constructed from a modified auxiliary protein may capture nucleotides and polynucleotides more easily than pores constructed from the wild type auxiliary protein. In addition, pore complexes constructed from the modified auxiliary protein may display an increased current range, which makes it easier to discriminate between different nucleotides, and a reduced variance of states, which increases the signal-to-noise ratio. In addition, the number of nucleotides contributing to the current as the polynucleotide moves through pore constructs comprising the modified auxiliary protein may be decreased. This makes it easier to identify a direct relationship between the observed current as the polynucleotide moves through the channel of the pore complex and the polynucleotide sequence. In addition, pore complexes constructed from the modified auxiliary protein may display an increased throughput, e.g., are more likely to interact with an analyte, such as a polynucleotide. This makes it easier to characterise analytes using the pore complexes. Pore complexes constructed from the modified auxiliary protein may insert into a membrane more easily, or may provide easier way to retain additional proteins in close vicinity of the pore complex.

In particular, pore complexes constructed from a modified nanopore may capture nucleotides and polynucleotides more easily than pores constructed from the wild type nanopore.

In addition, pore complexes constructed from the modified nanopore may display an increased current range, which makes it easier to discriminate between different nucleotides, and a reduced variance of states, which increases the signal-to-noise ratio. In addition, the number of nucleotides contributing to the current as the polynucleotide moves through pore constructs comprising the modified nanopore may be decreased. This makes it easier to identify a direct relationship between the observed current as the polynucleotide moves through the channel of the pore complex and the polynucleotide sequence. In addition, pore complexes constructed from the modified nanopore may display an increased throughput, e.g., are more likely to interact with an analyte, such as a polynucleotide. This makes it easier to characterise analytes using the pore complexes. Pore complexes constructed from the modified nanopore may insert into a membrane more easily, or may provide easier way to retain additional proteins in close vicinity of the pore complex.

### Method for Making Modified Proteins

Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis.

The transmembrane protein nanopore and auxiliary protein, or more specifically monomers or subunits thereof, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the monomer, or subunit, does not naturally contain such a sequence. An alternative to introducing a genetic tag is to chemically react a tag onto a native or engineered position on the protein. An example of this would be to react a gel-shift reagent to a cysteine engineered on the outside of the protein.

The monomer, or subunit, may be labelled with a revealing label. The revealing label may be any suitable label which allows the monomer, or subunit, to be detected. Suitable labels include, but are not limited to, fluorescent molecules, radioisotopes, e.g. ¹²⁵I, ³⁵S, enzymes, antibodies, antigens, polynucleotides and ligands such as biotin.

The transmembrane protein nanopore and/or auxiliary protein may, in one embodiment, be produced using D-amino acids. For instance, the transmembrane protein nanopore and/or auxiliary protein may comprise a mixture of L-amino acids and D-amino acids. This is conventional in the art for producing such proteins or peptides.

The transmembrane protein nanopore and/or auxiliary protein may comprise one or more specific modifications to facilitate nucleotide discrimination. The transmembrane protein nanopore and/or auxiliary protein may also contain other non-specific modifications as long as they do not interfere with pore formation. A number of non-specific side chain modifications are known in the art and may be made to the side chains of amino acids in the transmembrane protein nanopore and/or auxiliary protein. Such modifications include, for example, reductive alkylation of amino acids by reaction with an aldehyde followed by reduction with NaBH₄, amidination with methylacetimidate or acylation with acetic anhydride.

The transmembrane protein nanopore and/or auxiliary protein can be produced using standard methods known in the art. The transmembrane protein nanopore and/or auxiliary protein may be made synthetically or by recombinant means. For example, the proteins may be synthesised by in vitro translation and transcription (IVTT). The amino acid sequence of the protein may be modified to include non-naturally occurring amino acids or to increase the stability of the protein. When a protein is produced by synthetic means, such amino acids may be introduced during production. The protein may also be altered following either synthetic or recombinant production. Suitable methods for producing transmembrane protein nanopores are discussed in International applications WO 2010/004273, WO 2010/004265 or WO 2010/086603. Methods for inserting pores into membranes are known.

Polynucleotide sequences encoding a protein may be derived and replicated using standard methods in the art. Polynucleotide sequences encoding a protein may be expressed in a bacterial host cell using standard techniques in the art. The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, N.Y.

Proteins may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

Two or more monomers, or subunits, in the nanopore and/or auxiliary protein may be covalently attached to one another. For example, at least 2, at least 3, at least 4, at least 5, at least 6, at least 7, at least 8, at least 9 or at least 10 monomers, or subunits, may be covalently attached. The covalently attached monomers, or subunits, may be the same or different.

The monomers, or subunits, may be genetically fused, optionally via a linker, or chemically fused, for instance via a chemical crosslinker. Methods for covalently attaching monomers, or subunits, are disclosed in WO2017/149316, WO2017/149317 and WO2017/149318.

In some embodiments, the transmembrane protein nanopore and/or auxiliary protein is chemically modified. The transmembrane protein nanopore and/or auxiliary protein can be chemically modified in any way and at any site. The transmembrane protein nanopore and/or auxiliary protein may, for example, be chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The transmembrane protein nanopore and/or auxiliary protein may be chemically modified by the attachment of any molecule. For instance, the transmembrane protein nanopore and/or auxiliary protein may be chemically modified by attachment of a dye or a fluorophore.

Suitable chemical crosslinkers are well-known in the art. Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate. The most preferred crosslinker is succinimidyl 3-(2-pyridyldithio)propionate (SPDP). Typically, the molecule is covalently attached to the bifunctional crosslinker before the molecule/crosslinker complex is covalently attached to the mutant monomer but it is also possible to covalently attach the bifunctional crosslinker to the monomer before the bifunctional crosslinker/monomer complex is attached to the molecule. Suitable examples of peptide linkers are defined above.

The linker is preferably resistant to dithiothreitol (DTT). Suitable linkers include, but are not limited to, iodoacetamide-based and Maleimide-based linkers.

In other embodiment, the auxiliary protein and/or nanopore may be attached to a polynucleotide binding protein. This forms a modular sequencing system that may be used in the methods of sequencing of the invention. The polynucleotide binding protein may be covalently attached to the auxiliary protein and/or nanopore.

### Method of Producing Pore Complexes

The pore complex comprising an auxiliary protein and a transmembrane protein nanopore can, in one embodiment, be made via co-expression. Said method comprising the steps of expressing both pore monomers and the auxiliary protein, or auxiliary protein subunits or monomers, in a suitable host cell, and allowing in vivo complex pore formation. In this embodiment, at least one gene encoding a pore monomer in one vector and a gene encoding the auxiliary protein, or at least one auxiliary protein subunit or monomer in a second vector may be transformed together to express the proteins and make the complex within transformed cells. This is preferably carried out ex vivo or in vitro. Alternatively, the two genes encoding the pore monomer and auxiliary protein, or subunit thereof, can be placed in one vector under the control of a single promotor or under the control of two separate promoters, which may be the same or different.

Another method for producing the pore complex formed by the auxiliary protein and a transmembrane protein nanopore is in vitro reconstitution of proteins to obtain a functional pore. Said method comprises the steps of contacting the monomers of the transmembrane protein nanopore, with the auxiliary protein, or auxiliary protein subunits or monomers, in a suitable system to allow complex formation. Said system may be an “in vitro system”, which refers to a system comprising at least the necessary components and environment to execute said method, and makes use of biological molecules, organisms, a cell (or part of a cell) outside of their normal naturally-occurring environment, permitting a more detailed, more convenient, or more efficient analysis than can be done with whole organisms. An in vitro system may also comprise a suitable buffer composition provided in a test tube, wherein said protein components to form the complex have been added. A person skilled in the art is aware of the options to provide said system.

In this embodiment, the nanopore may be produced by expressing the monomer(s) separately from the auxiliary protein. Pore monomers or a nanopore may be purified from the cells transformed with a vector encoding at least one pore monomer, or with more than one vector each expressing a pore monomer. The auxiliary protein or subunits thereof may be purified from the cells transformed with a vector encoding at least one auxiliary protein subunit. The purified pore monomer(s)/nanopore may then be incubated together with the auxiliary protein or subunit(s) to make the pore complex.

In another embodiment, the nanopore monomer(s) and/or the auxiliary protein or subunit(s) thereof are produced separately by in vitro translation and transcription (IVTT). The nanopore monomer(s) may then be incubated together with the auxiliary protein or subunit(s) thereof to make the pore complex.

The above embodiments may be combined, such that for example, (i) the nanopore is produced in vivo and the auxiliary protein in vivo; (ii) the nanopore is produced in vitro and the auxiliary protein in vivo; (iii) the nanopore is produced in vivo and the auxiliary protein in vitro; or (iv) the nanopore is produced in vitro and the auxiliary protein in vitro.

One or both of the nanopore monomer and the auxiliary protein or subunit thereof may be tagged to facilitate purification. Purification can also be performed when the nanopore monomer and/or auxiliary protein or subunit thereof are untagged. Methods known in the art (e.g. ion exchange, gel filtration, hydrophobic interaction column chromatography etc.) can be used alone or in different combinations to purify the components of the pore complex.

Any known tags can be used in any of the two proteins. In one embodiment, two tag purification can be used to purify the pore complex from its component parts. For example, a Strep tag can be used in the nanopore and His tag can be used in the auxiliary protein or vice versa. A similar end result can be obtained when the two proteins are purified individually and mixed together followed by another round of Strep and His purification.

The pore complex can be made prior to insertion into a membrane or after insertion of the nanopore into a membrane. However, the nanopore may be inserted into a membrane and the auxiliary protein may be added afterwards so that the pore complex can form in situ. For example, in one embodiment, a system where the trans side or cis side of the membrane is accessible (for example in a chip or chamber for electrophysiology measurements), the nanopore may be inserted into the membrane, and then an auxiliary protein may be added from the trans side or cis side of the membrane, so that the complex can be formed in-situ.

In one embodiment, the auxiliary protein may comprise a protease cleavage site (e.g. TEV, HRV 3 or any other protease cleavage site), and be cleaved before or after associating with the nanopore. For example, a full length auxiliary protein (or subunits thereof) may be used to form the pore. Cleavage of amino acid residues that do not form part of the channel construction and are not required for interaction with the transmembrane pore may be cleaved from the auxiliary protein. In this embodiment, once the pore complex is formed, the protease is used to cleave the auxiliary protein. Alternatively, the protease may be used to produce the auxiliary protein prior to pore complex assembly.

Some protease sites will leave an additional tag behind after cleavage. For example, the TEV protease cleavage sequence is ENLYFQS. TEV protease cleaves the protein between Q and S leaving ENLYFQ intact at the C-terminus of the CsgF peptide. By way of another example, the HRV C3 cleavage site is LEVLFQGP and the enzyme cleaves between Q and G leaving LEVLFQ intact at the C-terminus of the CsgF peptide.

### System

In another aspect, the disclosure relates to a system for characterising a target polynucleotide, the system comprising a membrane and a pore complex;

wherein the pore complex comprises: (i) a nanopore located in the membrane, and (ii) an auxiliary protein or peptide attached to the nanopore;

wherein the nanopore and the auxiliary protein or peptide together form a continuous channel across the membrane, the channel comprising a first constriction region and a second constriction region;

wherein the first constriction region is formed by a portion of the nanopore, and wherein the second constriction region is formed by at least a portion of the auxiliary protein or peptide.

The pore complex, nanopore and auxiliary protein or peptide may be any as described herein above.

In one embodiment, the system further comprises a first chamber and a second chamber, wherein the first and second chambers are separated by the membrane. When used to characterise a target polynucleotide, the system may further comprise a target polynucleotide, wherein the target polynucleotide is transiently located within the continuous channel and wherein one end of the target polynucleotide is located in the first chamber and one end of the target polynucleotide is located in the second chamber.

In one embodiment, the system further comprises an electrically-conductive solution in contact with the nanopore, electrodes providing a voltage potential across the membrane, and a measurement system for measuring the current through the nanopore. In one embodiment, the voltage applied across the membrane and pore complex is from +5 V to −5 V, such as −600 mV to +600mV or −400 mV to +400 mV. The voltage used is preferably in the range 100 mV to 240 mV and more preferably in the range of 120 mV to 220 mV. It is possible to increase discrimination between different nucleotides by a pore by using an increased applied potential. Any suitable electrically-conductive solution may be used. For example, the solution may comprise charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In an exemplary system, salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane, e.g. in each chamber.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

A buffer may be present in the electrically-conductive solution. Typically, the buffer is phosphate buffer. Other suitable buffers are HEPES and Tris-HCl buffer. The pH of the electrically-conductive solution may be from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The system may comprise an array of pore complexes present in membranes. In a preferred embodiment, each membrane in the array comprises one pore complex. Due to the manner in which the array is formed, for example, the array may comprise one or more membrane that does not comprise a pore complex, and/or one or more membrane that comprises two or more pore complexes. The array may comprise from about 2 to about 1000, such as from about 10 to about 800, from about 20 to about 600 or from about 30 to about 500 membranes.

The system may be comprised in an apparatus. The apparatus may be any conventional apparatus for analyte analysis, such as an array or a chip. The apparatus is preferably set up to carry out the disclosed method. For example, the apparatus may comprise a chamber comprising an aqueous solution and a barrier that separates the chamber into two sections. The barrier typically has an aperture in which the membrane containing the pore is formed. Alternatively the barrier forms the membrane in which the pore is present.

In one embodiment, the apparatus comprises:

a sensor device that is capable of supporting the plurality of pores and membranes and being operable to perform analyte characterisation using the pores and membranes; and

at least one port for delivery of the material for performing the characterisation.

In one embodiment, the apparatus comprises:

a sensor device that is capable of supporting the plurality of pores and membranes being operable to perform analyte characterisation using the pores and membranes; and

at least one reservoir for holding material for performing the characterisation.

In one embodiment, the apparatus comprises:

a sensor device that is capable of supporting the membrane and plurality of pores and membranes and being operable to perform analyte characterising using the pores and membranes;

at least one reservoir for holding material for performing the characterising;

a fluidics system configured to controllably supply material from the at least one reservoir to the sensor device; and

one or more containers for receiving respective samples, the fluidics system being configured to supply the samples selectively from one or more containers to the sensor device.

The apparatus may also comprise an electrical circuit capable of applying a potential and measuring an electrical signal across the membrane and pore complex.

The apparatus may be any of those described in WO 2008/102120, WO 2009/077734, WO 2010/122293, WO 2011/067559 or WO 00/28312.

### Membrane

Any suitable membrane may be used in the system. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphipiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

The membrane is most preferably one of the membranes disclosed in International Application No. WO2014/064443 or WO2014/064444.

The amphiphilic molecules may be chemically-modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In a preferred embodiment, the lipid bilayer is formed as described in International Application No. WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another preferred embodiment, the membrane comprises a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### Methods of Characterising an Analyte

In a further aspect, a method of determining the presence, absence or one or more characteristics of a target analyte is disclosed. The method involves contacting the target analyte with a membrane comprising a pore complex, such that the target analyte moves with respect to, such as into or through, the continuous channel comprising at least two constructions provided by a nanopore and an auxiliary protein or peptide in the pore complex, respectively, and taking one or more measurements as the analyte moves with respect to the channel and thereby determining the presence, absence or one or more characteristics of the analyte. The analyte may pass through the nanopore constriction, followed by the auxiliary protein constriction. In an alternative embodiment the analyte may pass through the auxiliary protein constriction, followed by the nanopore constriction, depending on the orientation of the pore complex in the membrane.

In one embodiment, the method is for determining the presence, absence or one or more characteristics of a target analyte. The method may be for determining the presence, absence or one or more characteristics of at least one analyte. The method may concern determining the presence, absence or one or more characteristics of two or more analytes. The method may comprise determining the presence, absence or one or more characteristics of any number of analytes, such as 2, 5, 10, 15, 20, 30, 40, 50, 100 or more analytes. Any number of characteristics of the one or more analytes may be determined, such as 1, 2, 3, 4, 5, 10 or more characteristics.

The binding of a molecule in the channel of the pore complex, or in the vicinity of either opening of the channel will have an effect on the open-channel ion flow through the pore, which is the essence of “molecular sensing” of pore channels. In a similar manner to the nucleic acid sequencing application, variation in the open-channel ion flow can be measured using suitable measurement techniques by the change in electrical current (for example, WO 2000/28312 and D. Stoddart et al., Proc. Natl. Acad. Sci., 2010, 106, 7702-7 or WO 2009/077734). The degree of reduction in ion flow, as measured by the reduction in electrical current, is related to the size of the obstruction within, or in the vicinity of, the pore. Binding of a molecule of interest, also referred to as an “analyte”, in or near the pore therefore provides a detectable and measurable event, thereby forming the basis of a “biological sensor”. Suitable molecules for nanopore sensing include nucleic acids; proteins; peptides; polysaccharides and small molecules (refers here to a low molecular weight (e.g., <900 Da or <500 Da) organic or inorganic compound) such as pharmaceuticals, toxins, cytokines, and pollutants. Detecting the presence of biological molecules finds application in personalised drug development, medicine, diagnostics, life science research, environmental monitoring and in the security and/or the defence industry.

The target analyte may be a metal ion, an inorganic salt, a polymer, an amino acid, a peptide, a polypeptide, a protein, a nucleotide, an oligonucleotide, a polynucleotide, a polysaccharide, a dye, a bleach, a pharmaceutical, a diagnostic agent, a recreational drug, an explosive, a toxic compound, or an environmental pollutant. The method may concern determining the presence, absence or one or more characteristics of two or more analytes of the same type, such as two or more proteins, two or more nucleotides or two or more pharmaceuticals. Alternatively, the method may concern determining the presence, absence or one or more characteristics of two or more analytes of different types, such as one or more proteins, one or more nucleotides and one or more pharmaceuticals.

The target analyte can be secreted from cells. Alternatively, the target analyte can be an analyte that is present inside cells such that the analyte must be extracted from the cells before the method can be carried out.

In one embodiment, the analyte is an amino acid, a peptide, a polypeptides or protein. The amino acid, peptide, polypeptide or protein can be naturally-occurring or non-naturally-occurring. The polypeptide or protein can include within them synthetic or modified amino acids. Several different types of modification to amino acids are known in the art. Suitable amino acids and modifications thereof are above. It is to be understood that the target analyte can be modified by any method available in the art.

In a preferred embodiment, the analyte is a polynucleotide, such as a nucleic acid. A polynucleotide is defined as a macromolecule comprising two or more nucleotides. The naturally-occurring nucleic acid bases in DNA and RNA may be distinguished by their physical size. As a nucleic acid molecule, or individual base, passes through the channel of a nanopore, the size differential between the bases causes a directly correlated reduction in the ion flow through the channel. The variation in ion flow may be recorded. Suitable electrical measurement techniques for recording ion flow variations are described in, for example, WO 2000/28312 and D. Stoddart et al., Proc. Natl. Acad. Sci., 2010, 106, pp 7702-7 (single channel recording equipment); and, for example, in WO 2009/077734 (multi-channel recording techniques). Through suitable calibration, the characteristic reduction in ion flow can be used to identify the particular nucleotide and associated base traversing the channel in real-time. In typical nanopore nucleic acid sequencing, the open-channel ion flow is reduced as the individual nucleotides of the nucleic sequence of interest sequentially pass through the channel of the nanopore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, it has typically required for the reduction in ion flow through the channel to be directly correlated to the size of the individual nucleotide passing through the constriction (or “reading head”). It will be appreciated that sequencing may be performed upon an intact nucleic acid polymer that is ‘threaded’ through the pore via the action of an associated polymerase or helicase, for example. Alternatively, sequences may be determined by passage of nucleotide triphosphate bases that have been sequentially removed from a target nucleic acid in proximity to the pore (see for example WO 2014/187924).

The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag, for which suitable examples are known by a skilled person. The polynucleotide may comprise one or more spacers. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. At least a portion of the polynucleotide is preferably double stranded. The polynucleotide is most preferably ribonucleic nucleic acid (RNA) or deoxyribonucleic acid (DNA). In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii), and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e. lack a nucleobase). A nucleotide may also lack a nucleobase and a sugar (i.e. is a C3 spacer). The sequence of the nucleotides (iii) is determined by the consecutive identity of following nucleotides attached to each other throughout the polynucleotide strain, in the 5′ to 3′ direction of the strand.

The pore complexes comprising at least two reader heads are particularly useful in analysing homopolymers. For example, the pores may be used to determine the sequence of a polynucleotide comprising two or more, such as at least 3, 4, 5, 6, 7, 8, 9 or 10, consecutive nucleotides that are identical. For example, the pores may be used to sequence a polynucleotide comprising a polyA, polyT, polyG and/or polyC region.

For example, the CsgG pore constriction is made of the residues at the 51, 55 and 56 positions of SEQ ID NO: 3. The reader head of CsgG and its constriction mutants are generally sharp. When DNA is passing through the constriction, interactions of approximately 5 bases of DNA with the reader head of the pore at any given time dominate the current signal. Although these sharper reader heads are very good in reading mixed sequence regions of DNA (when A, T, G and C are mixed), the signal becomes flat and lacks some information when there is a homopolymeric region within the DNA (eg: polyT, polyG, polyA, polyC). Because 5 bases dominate the signal of the CsgG and its constriction mutants, it is difficult to discriminate homopolymers longer than 5 without using additional dwell time information. However, if DNA is passing through a second reader head, more DNA bases will interact with the combined reader heads, increasing the length of the homopolymers that can be discriminated. The Examples and Figures show that such an increase in homopolymer sequencing accuracy is achieved using the pore comprising a CsgG pore and a second reader head.

### Kit

In a further aspect, the present invention also provides a kit for characterising a target polynucleotide. The kit comprises the disclosed pore complex, and the components of a membrane. The membrane is preferably formed from the components. The pore complex is preferably present in the membrane, together forming a transmembrane pore complex channel. The kit may comprise components of any type of membranes, such as an amphiphilic layer or a triblock copolymer membrane. The kit may further comprise a polynucleotide binding protein, such as a nucleic acid handling enzyme, for example a polymerase or a helicase. The kit may further comprise one or more anchors, such as cholesterol, for coupling the polynucleotide to the membrane. The kit may further comprise one or more polynucleotide adaptors that can be attached to a target polynucleotide to facilitate characterisation of the polynucleotide. In one embodiment, the anchor, such as cholesterol, is attached to the polynucleotide adaptor. The kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used. Finally, the kit may also comprise additional components useful in polynucleotide characterization.

It is to be understood that although particular embodiments, specific configurations as well as materials and/or molecules, have been discussed herein for engineered cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1: Double Pore Production

DNA (SEQ ID NO: 89) encoding the polypeptide Pro-CP1-Eco-(Mutant-StrepII(C)) (SEQ ID NO: 90) was cloned into a pT7 vector containing ampicillin resistance gene. Concentration of DNA solution was adjusted to 400 μg/μL. 1 μl of DNA was used to transform the cell line ONT001 which is Lemo BL21 DE3 cell line in which the gene coding for CsgG protein is replaced with DNA responsible for kanamycin resistance. Cells were then plated out on LB agar containing ampicillin (0.1 mg/ml) and kanamycin (0.03 mg/ml) and incubated for approximately 16 hours at 37° C.

Bacterial colonies grown on LB plates containing ampicillin and kanamycin can be assumed to have incorporated the CP1 plasmid with no endogenous production. One such colony was used to inoculate a starter culture of LB media (100 mL) containing both carbenicillin (0.1 mg/ml) and kanamycin (0.03 mg/ml). The starter culture was grown at 37° C. with agitation, until OD600 was reached to 1.0-1.2. The starter culture was used to inoculate a fresh 500 ml culture to and OD600 of 0.1. LB media containing the following additives—carbenicillin (0.1 mg/ml), kanamycin (0.03 mg/ml), 500 μM Rhamnose, 15 mM MgSO4 and 3 mM ATP. The culture was grown at 37° C. with agitation until stationary phase was entered and held for a further hour—stationary phase ascertained by plateau of measured OD600. Temperature of the culture was then adjusted to 18° C. and glucose was added to a final concentration of 0.2%. Once culture was stable at 18° C. induction was initiated by the addition of lactose to a final concentration of 1%. Induction was carried out for approximately 18 hours with agitation at 18° C.

Following induction, the culture was pelleted by centrifugation at 6,000 g for 30 minutes. The pellet was resuspended in 50 mM Tris, 300 mM NaCl, containing Protease Inhibitors (Merck Millipore 539138), Benzonase Nuclease (Sigma E1014), 1× Bugbuster (Merck Millipore 70921) and 0.1% Brij 58 pH8.0 (approximately 10 ml of buffer per gram of pellet). The suspension was mixed well until it is fully homogeneous, sample was then transferred to roller mixer at 4° C. for approximately 5 hours. Lysate was pelleted by centrifugation at 20,000 g for 45 minutes and the supernatant was filtered through 0.22 μM PES syringe filter. Supernatant which contains CP1 was taken forward for purification by column chromatography.

Sample was applied to a 5m1 Strep Trap column (GE Healthcare). Column was washed with 25 mM Tris, 150 mM NaCl, 2 mM EDTA, 0.1% Brij 58 pH8 until a stable baseline of 10 column volumes was maintained. Column was then washed with 25 mM Tris, 2M NaCl, 2 mM EDTA, 0.1% Brij 58 pH8 before being returned to 150 mM buffer. Elution was carried out with 10 mM desthiobiotin. Elution peak was pooled and carried forward for ion exchange purification on a 1 ml Q HP column (GE Healthcare) using 25 mM Tris, 150 mM NaCl, 2 mM EDTA, 0.1% Brij 58 pH8 as the binding buffer and 25 mM Tris, 500 mM NaCl, 2 mM EDTA, 0.1% Brij 58 pH8 as the elution buffer. Flowthrough peak was observed to contain both dimer and monomer protein, elution peak at approx. 400 ms/sec was observed to contain monomeric pore. Flowthrough peak was concentrated via vivaspin column (100kd MWCO) and carried forward for size exclusion chromatography on 24 ml S200 increase column (GE Healthcare) with the buffer 25 mM Tris, 150 mM NaCl, 2 mM EDTA, 0.1% Brij 58, 0.1% SDS pH8. Dimeric (double) pore eluted at 9 ml while the monomeric pore eluted at 10.5 ml.

### Example 2: CsgG:CsgF Complex Protein Production (Co-Expression, In Vitro Reconstitution, Coupled In-Vitro Transcription and Translation and Reconstitution of CsgG with CsgF Synthetic Peptides)

To produce the CsgG:CsgF complex, both proteins can be co-expressed in a suitable Gram-negative host such as E. coli, and extracted and purified as a complex from the outer membrane. The in vivo formation of the CsgG pore and the CsgG:CsgF complex requires targeting of the proteins to the outer membrane. To do so, CsgG is expressed as a prepro-protein with a lipoprotein signal peptide (Juncker et al. 2003, Protein Sci. 12(8): 1652-62) and Cys residue at the N-terminal position of the mature protein (SEQ ID No:3). An example of such lipoprotein signal peptide is residues 1-15 of full length E. coli CsgG as shown in SEQ ID No:2.

Processing of prepro CsgG results in cleavage of the signal peptide and lipidatation of mature CsgG, following by transfer of the mature lipoprotein to the outer membrane, where it inserts as an oligomeric pore (Goyal et al. 2014, Nature 516(7530):250-3). To form the CsgG:CsgF complex, CsgF can be co-expressed with CsgG and targeted to the periplasm by means of a leader sequence such as the native signal peptide corresponding to residues 1-19 of SEQ ID No:5. CsgG:CsgF combination pores can then be extracted from the outer membrane using detergents, and purified to a homogeneous complex by chromatography.

Alternatively, the CsgG:CsgF pore complex can be produced by in vitro reconstitution using the CsgG pore and CsgF—see below.

For in vivo CsgG:CsgF complex formation, E. coli CsgF (SEQ ID NO:5) and CsgG (SEQ ID NO:2) were co-expressed using their native signal peptides to ensure periplasmic targeting of both proteins, as well as N-terminal lipidation of CsgG. Additionally, for ease of purification, CsgF was modified by introduction of a C-terminally 6x histidine tag and CsgG was fused C-terminally to a Strep-II tag. Co-expression and complex purification was performed as described in the Methods. SDS-PAGE analysis of the His affinity purification eluate revealed the enrichment of CsgF-His, as well as the co-purification of CsgG-Strep, suggesting the latter was in a complex with CsgF. Additionally, the SDS-PAGE revealed that a significant fraction of the eluted CsgF ran at lower molecular mass due to the loss of a N-terminal fragment of the protein. SDS-PAGE analysis of the pooled fractions of the His-trap elution of the second affinity purification revealed the presence of CsgG and CsgF in an apparent equimolar concentrations, as well as the loss of the CsgF truncation fragment seen in the His-trap eluate. Co-elution of CsgF in the Strep-affinity purification indicated that the protein is present as a non-covalent complex with CsgG. Strikingly, the N-terminal truncation fragment of CsgF was lost in the Strep-affinity purification, suggesting that the CsgF N-terminus is required to bind CsgG.

To produce the CsgG:CsgF complex by in vitro reconstitution, CsgG and CsgF were expressed in separate E. coli cultures transformed with pPG1 and pNA101, respectively, and purified, followed by in vitro reconstitution of the CsgG:CsgF complex (see Methods). For comparison, purified CsgG was similarly run over the Superose 6 column as the complex. The CsgG Superose 6 run showed the existence of two discrete populations, corresponding to nonameric CsgG pores as well as dimers of nonameric CsgG pores, as previously described in Goyal et al. (2014). The Superose 6 run of the CsgG:CsgF reconstitution revealed the existence of three discrete populations corresponding to excess CsgF, nonameric CsgG:CsgF complex and dimers of nonameric CsgG:CsgF. To provide independent confirmation of the formation of CsgG:CsgF complexes, the various Superose 6 elution peaks were analysed on native PAGE.

Surprisingly, CsgG:CsgF complex can also be made by coupled in vitro transcription and translation (IVTT) method as described in the materials and methods section for characterisation of analytes. The complex can be made either by expressing CsgG and CsgF proteins in the same IVTT reaction or reconstituting separately made CsgG and CsgF in two different IVTT reactions. In one example, E. coli T7-S30 extract system for circular DNA (Promega) has been used to make the CsgG:CsgF complex in one reaction mixture and proteins were analysed on SDS-PAGE. Since the protein expression in IVTT does not use the natural molecular machinery of protein expression, DNA that are used to express proteins in IVTT lack the DNA encoding the signal peptide region. When the DNA of CsgG is expressed in IVTT in the absence of DNA of CsgF, only the monomers of CsgG can be produced. Surprisingly, these expressed monomers can be assembled into CsgG oligomeric pores in situ by using cell extract membranes present in the IVTT reaction mixture. Although the oligomer of CsgG is SDS stable, it breaks down into its constituent monomers when the sample is heated to 100° C. When the DNA of CsgF is expressed in IVTT in the absence of DNA of CsgG, only CsgF monomers can be seen. When DNA of CsgG and CsgF are mixed in 1:1 ratio and expressed simultaneously in the same IVTT reaction mixture, CsgF proteins generated interact with the assembled CsgG pore with high efficiency to make CsgG:CsgF complex. This SDS stable complex made in IVTT is heat stable at least up to 70° C.

CsgG:CsgF complexes with truncated CsgF can also be made by any of the methods shown above by using DNA encoding truncated CsgF instead of the full length version. However, stability of the complex may be compromised when CsgF is truncated below the FCP domain. In addition, CsgG:CsgF complexes with truncated CsgF can be made by cleaving the full length CsgF in appropriate positions once the full length CsgG:CsgF complex is formed. Truncations can be done by modifying the DNA that encode CsgF protein by incorporating protease cleavage sites at positions where cleavage is needed. Seq ID No. 56-67 show TEV or HCV C3 protease sites incorporated in various positions of CsgF to generate CsgG:CsgF complexes with truncated CsgF. When the CsgG:CsgF complex (with full length CsgF) is treated with TEV protease enzyme as described in the materials and methods section for characterisation of analytes, CsgF is being truncated at position 35. However, TEV cleavage leaves an extra 6 amino acids at the C terminal of the cleavage site. Therefore, remaining CsgF truncated protein in complex with the CsgG pore is 42 amino acids long. Molecular weight difference of this complex and the CsgG pore (without the CsgF) is still visible in SDS-PAGE.

Surprisingly, CsgG:CsgF complexes with truncated CsgF can also be made by reconstituting purified CsgG pore (made by in vivo or in vitro) with synthetic peptides of appropriate length. Since the reconstitution takes place in vitro, signal peptide of CsgF is not required to make the CsgG:CsgF complex. Further, this method does not leave extra amino acids at the C terminus of the CsgF. Mutations and modifications can also be easily incorporated into synthetic CsgF peptides. Therefore, this method is a very convenient way to reconstitute different CsgG pores or mutants or homologues thereof with different CsgF peptides or mutants or homologues thereof to generate different CsgG:CsgF complex variants. Stability of the complex may be compromised when the CsgF is truncated beyond the FCP domain. Surprisingly, SDS-PAGE analysis of the heat stability of CsgG:CsgF complexes made by this method with CsgF-(1-45) (FIG. 13.A), CsgF-(1-35) (FIG. 13.B) and CsgF-(1-30) (FIG. 13.C) shows at least CsgF-(1-45) and CsgF-(1-35) peptides make complexes with CsgG that are heat stable at least to 90° C. Since the CsgG pore breaks down to its constituent monomers at 90° C., it is difficult to assess the stability of the complex beyond 90° C. Due to the minimal difference between the CsgG pore band and the CsgG:CsgF-(1-30) complex band in SDS-PAGE, this method is not sufficient to analyse the heat stability of the CsgG:CsgF-(1-30) complex (FIG. 13.C). However, CsgG:CsgF complexes have been observed in all three cases and even with CsgG:CsgF-(1-29) in electrophysiological experiments indicating that even CsgF-(1-29) peptide is producing at least some CsgG:CsgF complexes (FIG. 21).

### Example 3: CsgG:CsgF Structural Analysis via cryo-EM

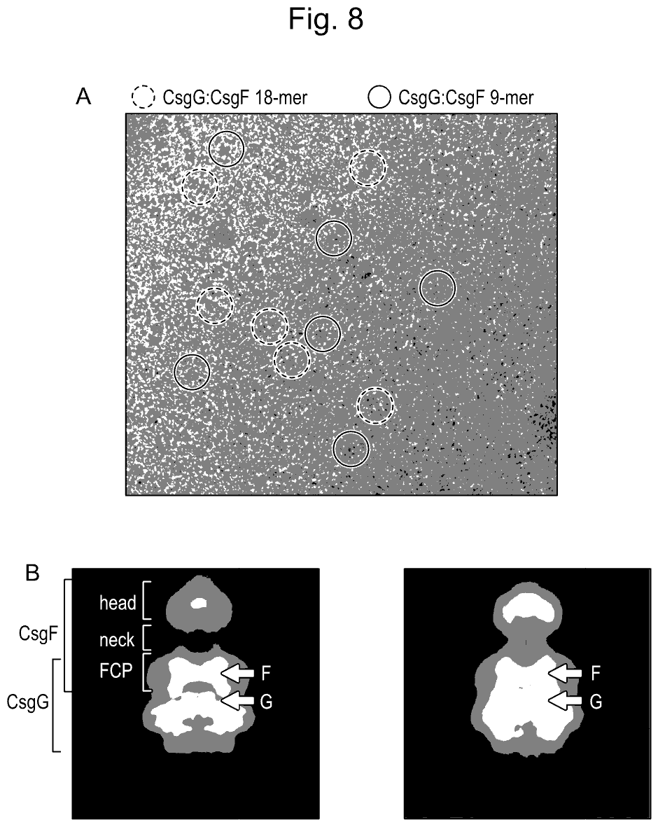

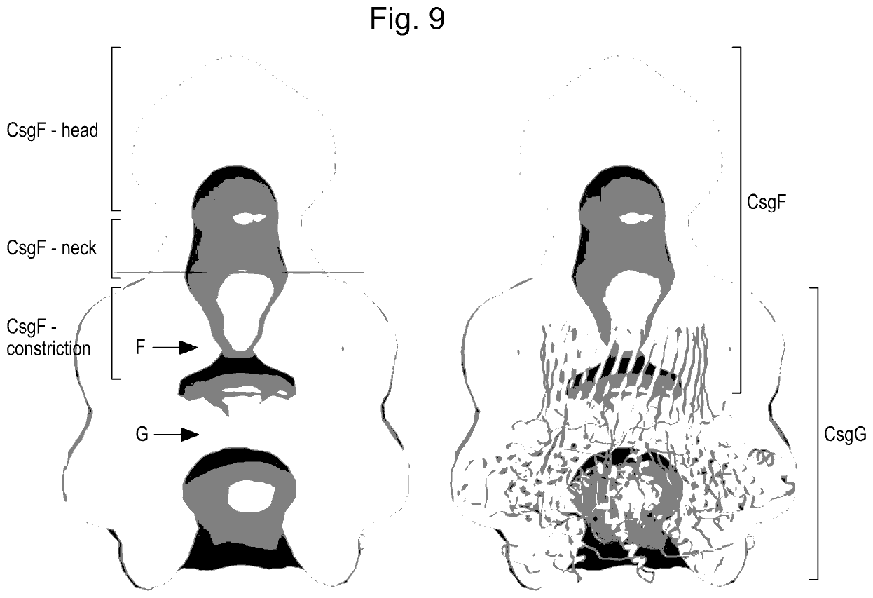

To gain structural insight in the CsgG:CsgF complex, co-purified or in vitro reconstituted CsgG:CsgF particles were analysed by transmission electron microscopy. In preparation of cryo-EM analysis, 500 μL of the peak fraction of the double-affinity purified CsgG:CsgF complex was injected onto a Superose 6 10/30 column (GE Healthcare) equilibrated with Buffer D (25 mM Tris pH8, 200 mM NaCl and 0.03% DDM), and run at 0.5 mL/min. Protein concentration was determined based on calculated absorbance at 280 nm and assuming 1:1 stoichiometry. Samples for electron cryomicroscopy were analysed as described in the Methods. A cryo-EM micrograph of the CsgG:CsgF complex as well as two selected class averages from the picked CsgG:CsgF particles are shown in FIG. 8. The micrograph shows the presence of nonameric pore as well as dimer of nonameric pore complexes. For image reconstruction, nonameric CsgG:CsgF particles were picked and aligned using RELION. Class averages of the CsgG:CsgF complex as side views, as well as the 3D reconstructed electron density show the presence of an additional density corresponding to CsgF, seen as a protrusion from the CsgG particle, located at the side of the CsgG β-barrel (FIG. 8B, 9). The additional density reveals three distinct regions, encompassing a globular head domain, a hollow neck domain and a domain that interacts with the CsgG β-barrel. The latter CsgF region, referred to as CsgF constriction peptide or FCP, inserts into the lumen of the CsgG β-barrel and can be seen to form an additional constriction (labeled F in FIG. 8B, 5) of the CsgG pore, located approximately 2 nm above the constriction formed by the CsgG constriction loop (labeled G in FIG. 8B, 5).

### Example 4: Identification of the CsgF Interaction and Constriction Peptide by Truncation of CsgF

The presence of a second constriction in the CsgG:CsgF pore complex as compared to the CsgG only pore provides opportunities for nanopore sensing applications, providing a second orifice in the nanopore that can be used as a second reader head or as an extension of the primary reader head provided by the CsgG constriction loop. However, when in complex with the full length CsgF, the exit side of CsgG:CsgF combination pore is blocked by the CsgF neck and head domains. Therefore, we sought to determine the CsgF region required to interact with and insert into the CsgG β-barrel. Our Strep-tactin affinity purification experiments hinted that the N-terminal region of CsgF was required for CsgG interaction, since an N-terminal truncation fragment of CsgF present in the His-trap affinity purification was lost and did not co-purify with CsgG. CsgF homologues are characterised by the presence of PFAM domain PF03783. When performing a multiple sequence alignment (MSA) of CsgG homologues found in Gram-negative bacteria, a region of sequence conservation (between 35 and 100% pairwise sequence identity) was seen corresponding to the first ˜30-35 amino acids of mature CsgF (SEQ ID NO:6). Based on the combined data, this N-terminal region of CsgF was hypothesised to form the CsgG interaction peptide or FCP.

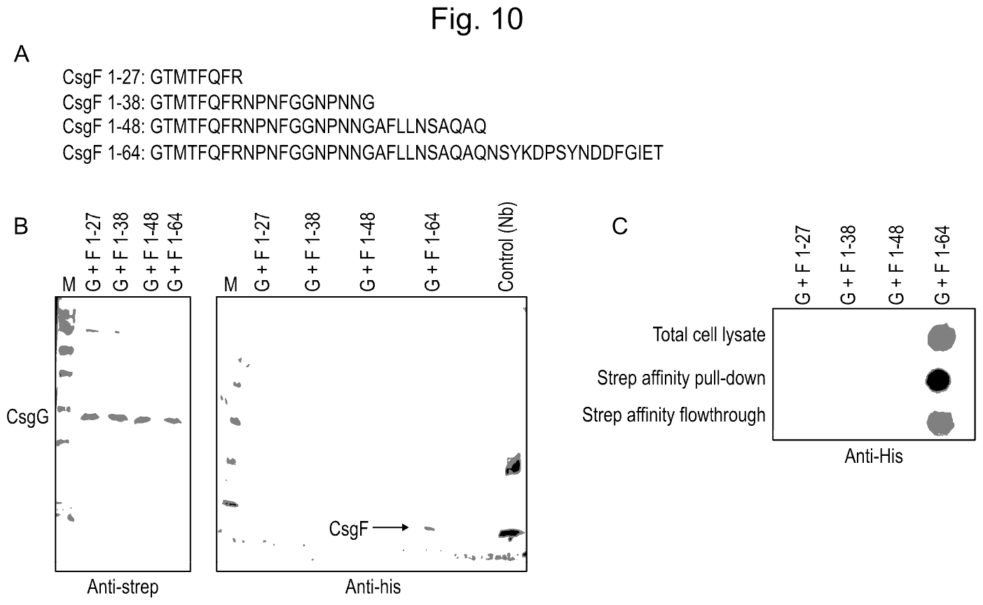

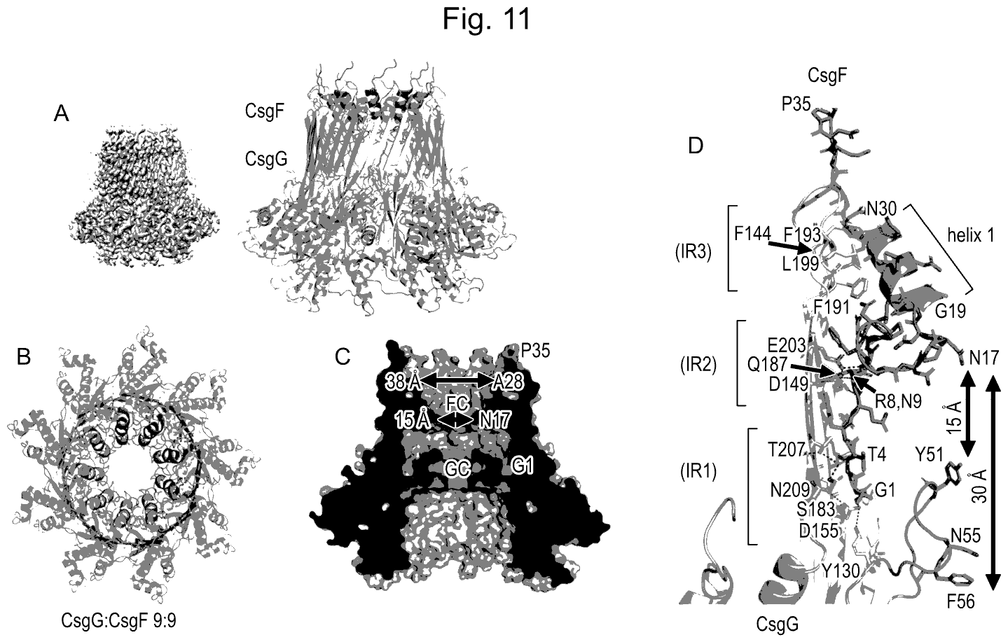

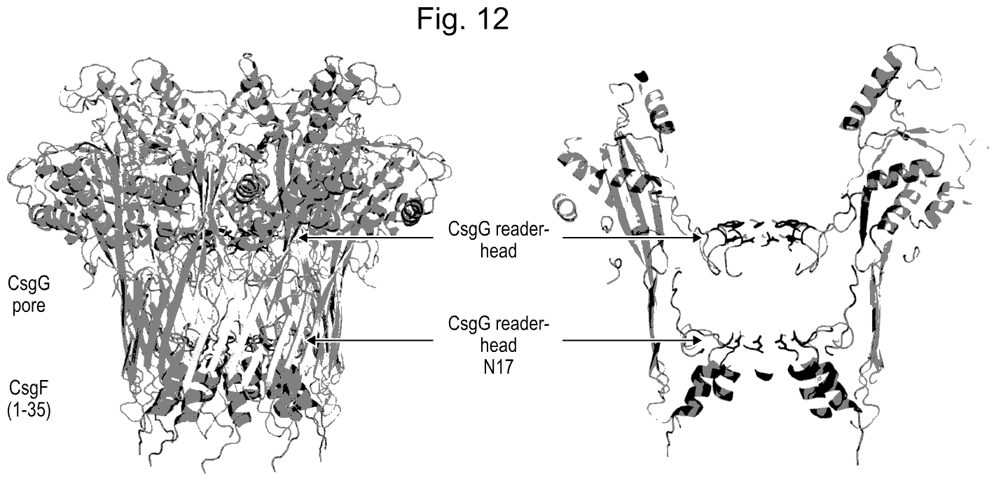

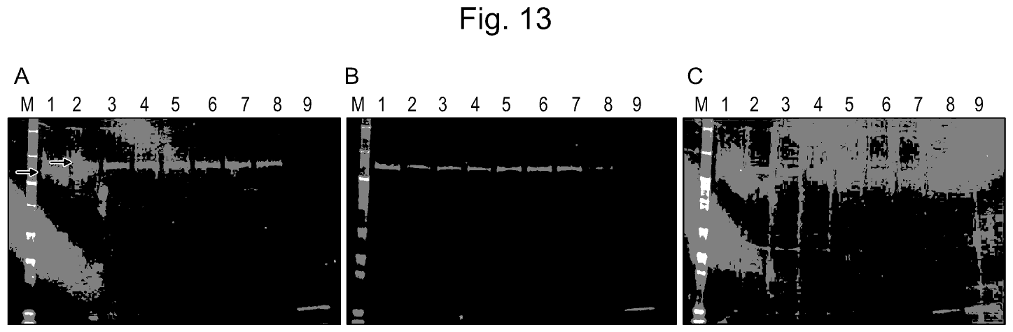

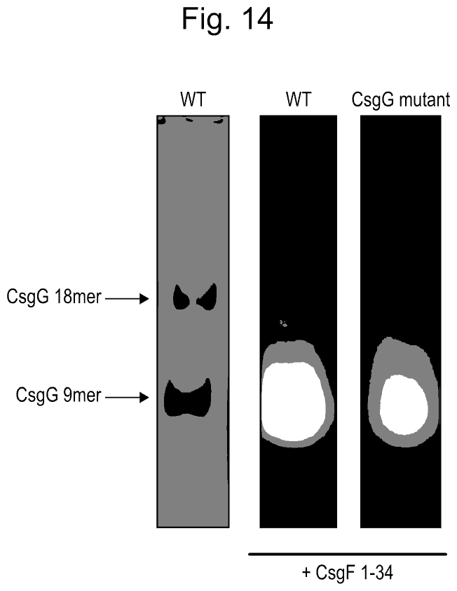

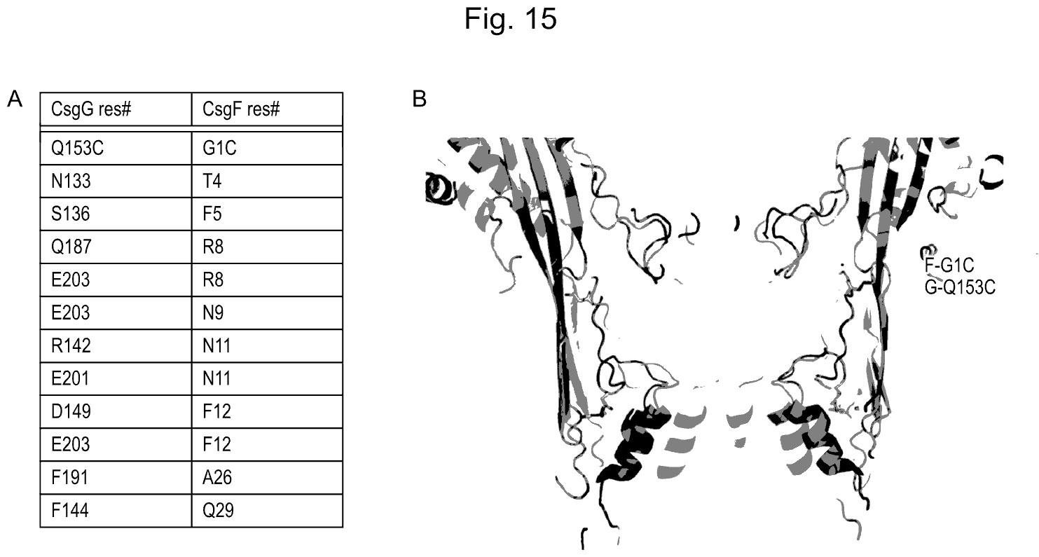

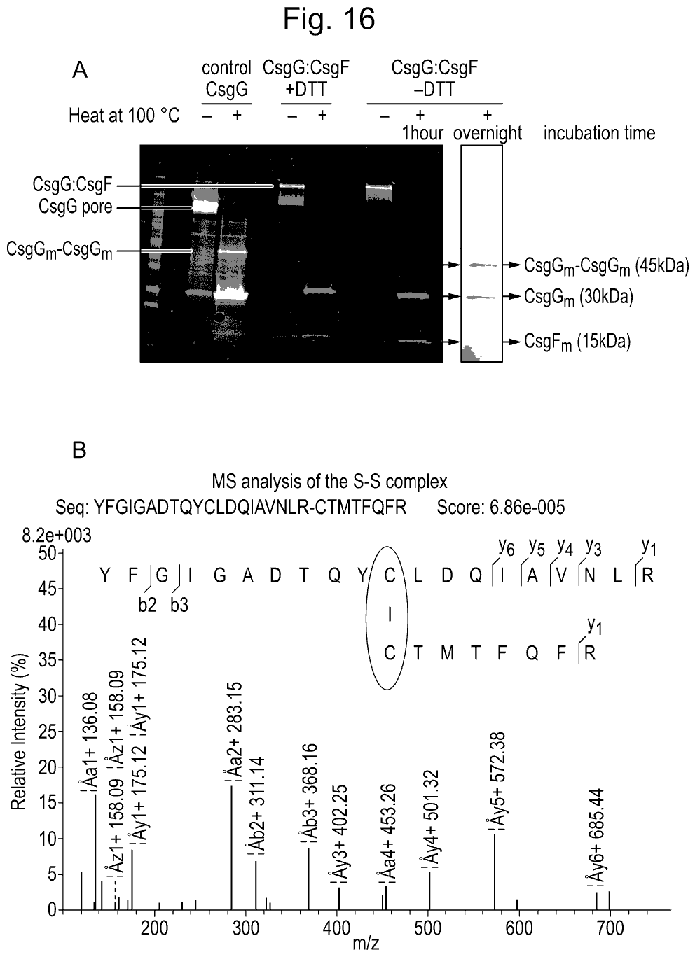

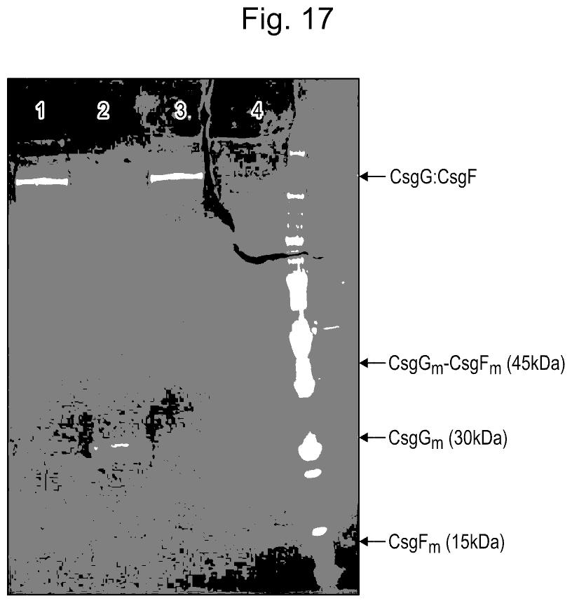

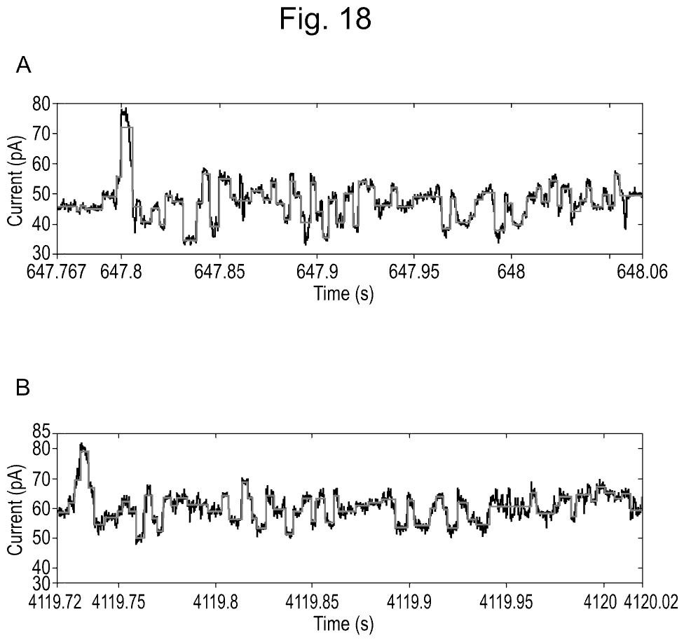

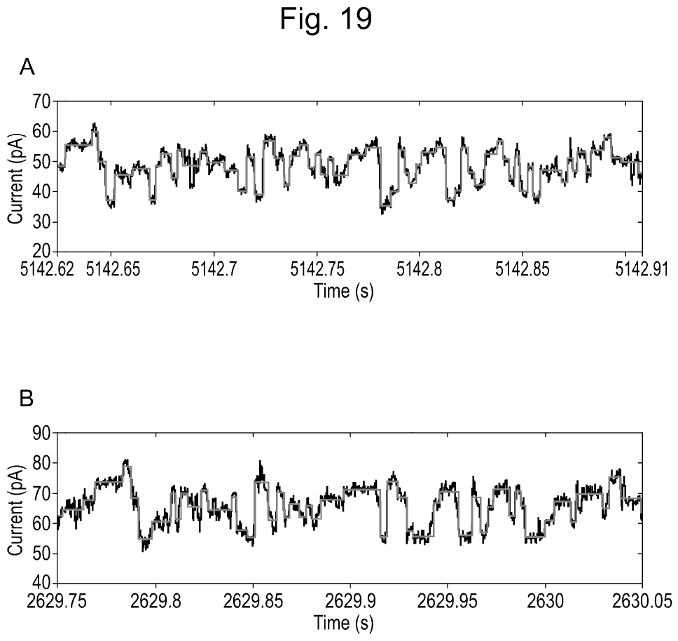

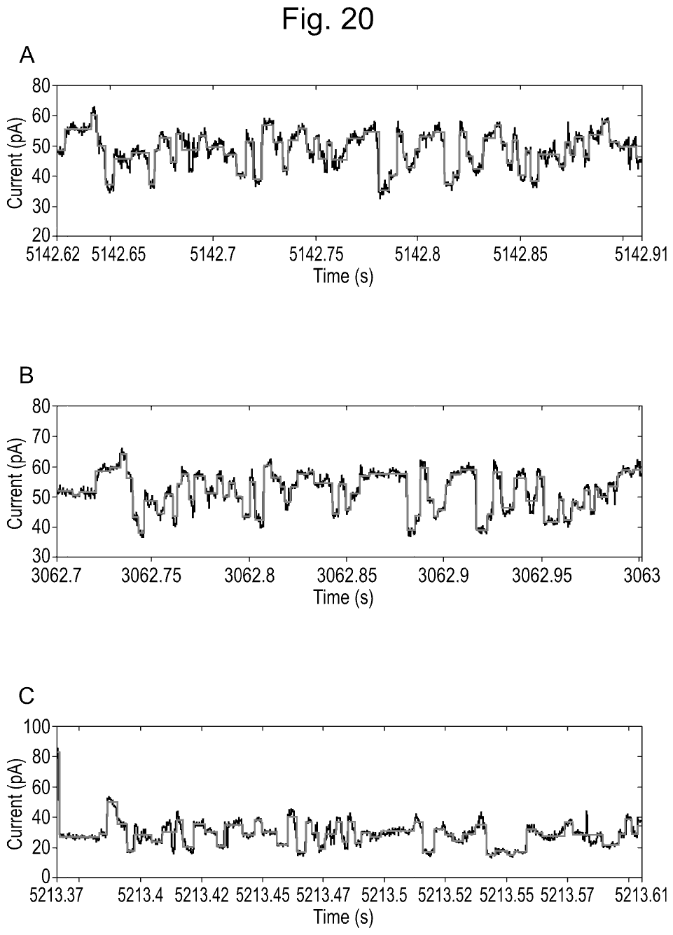

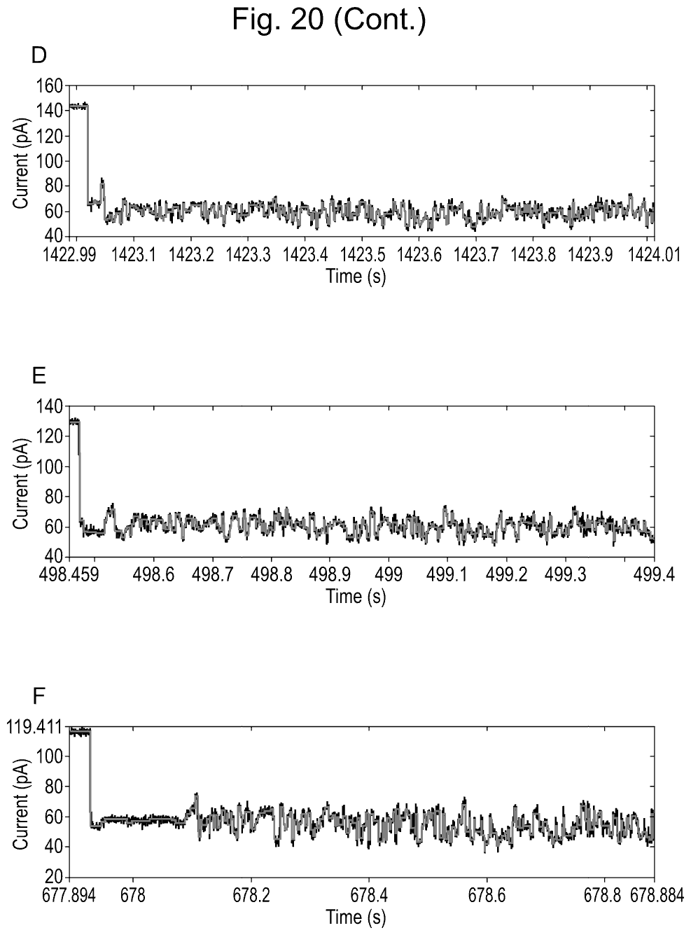

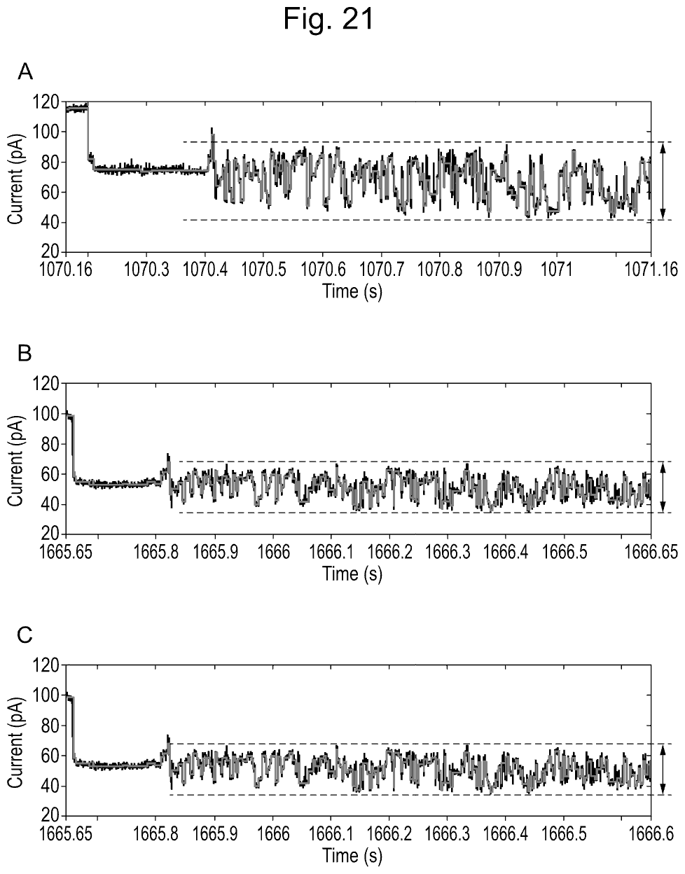

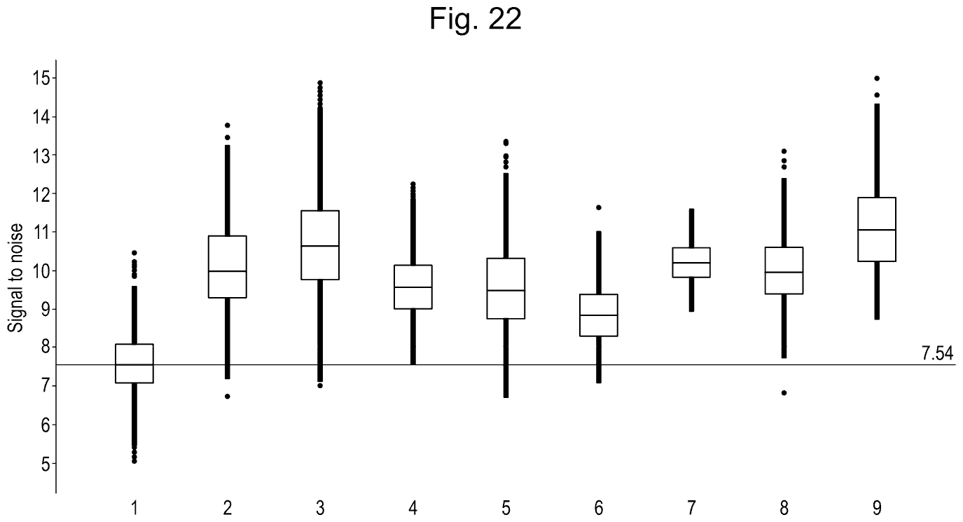

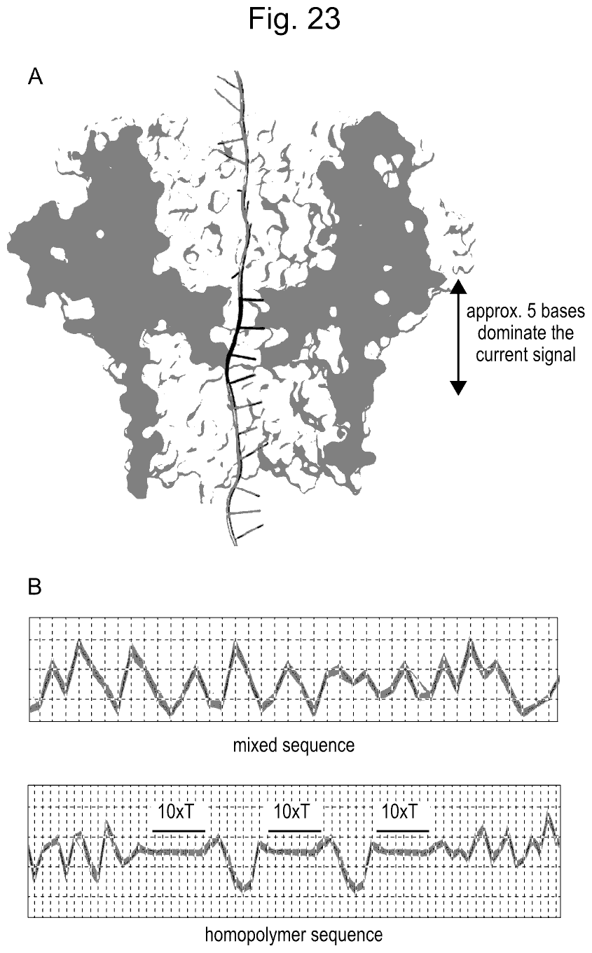

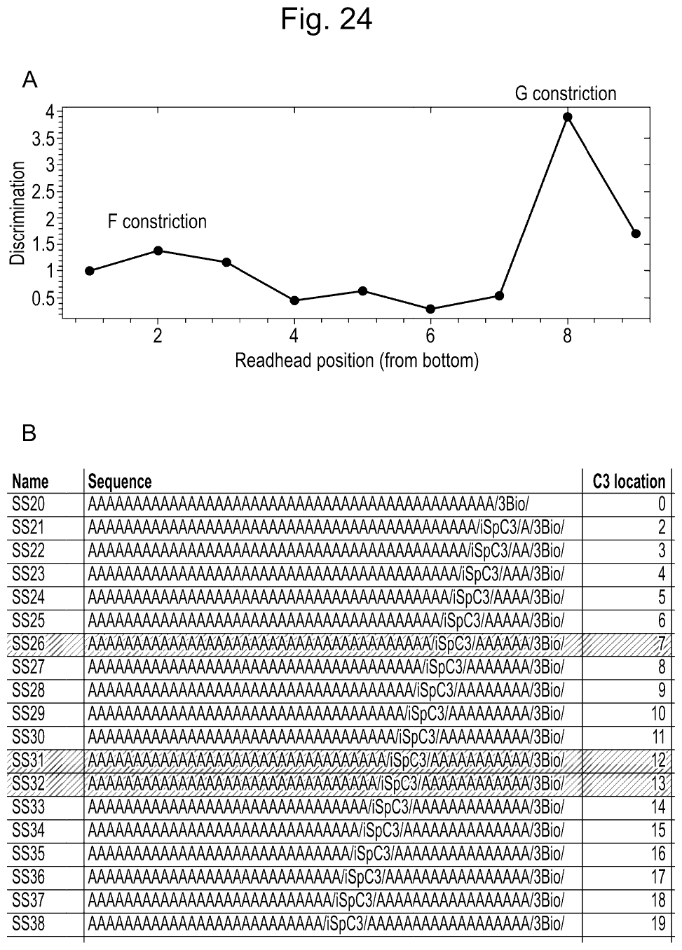

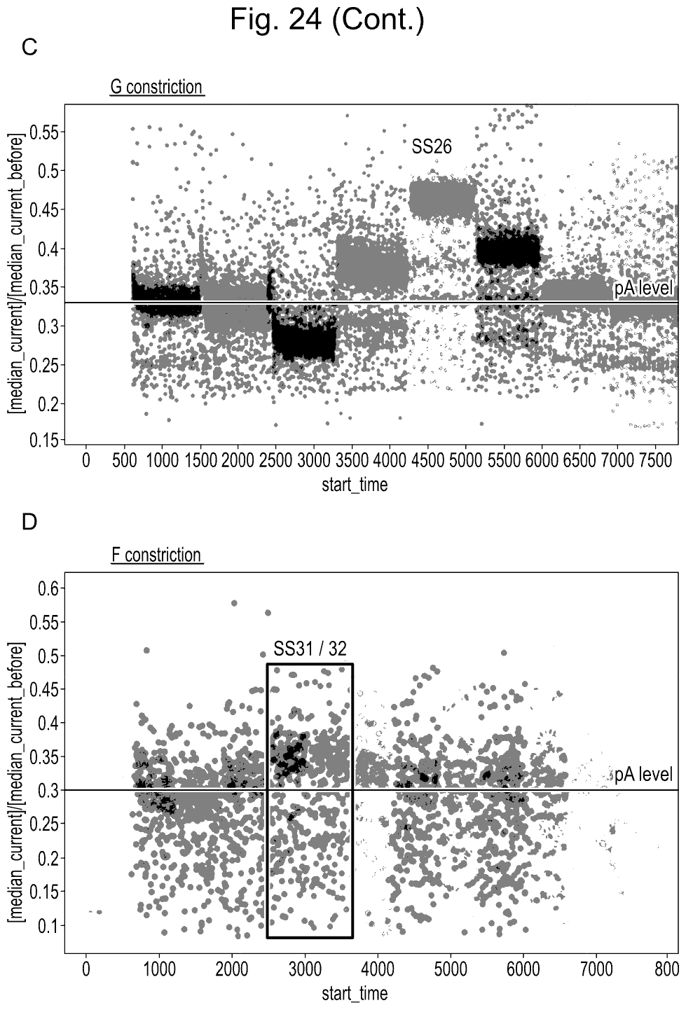

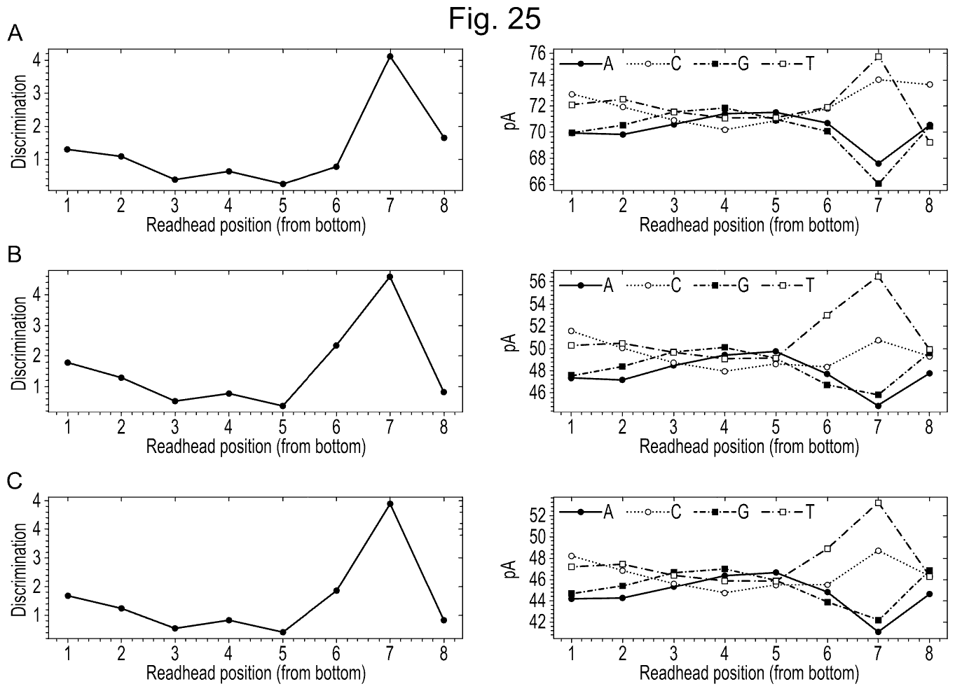

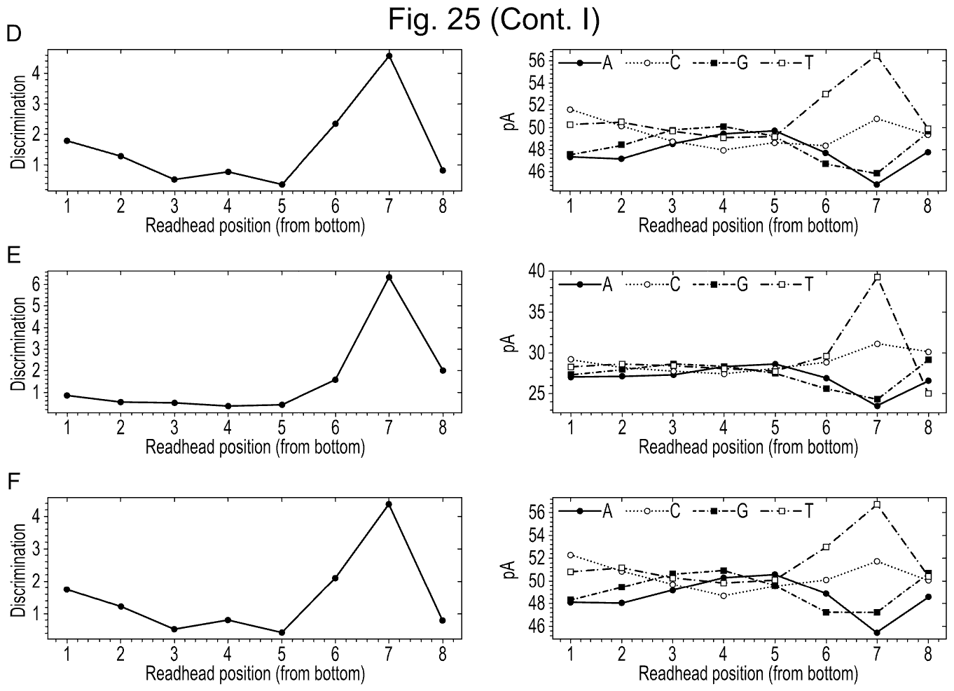

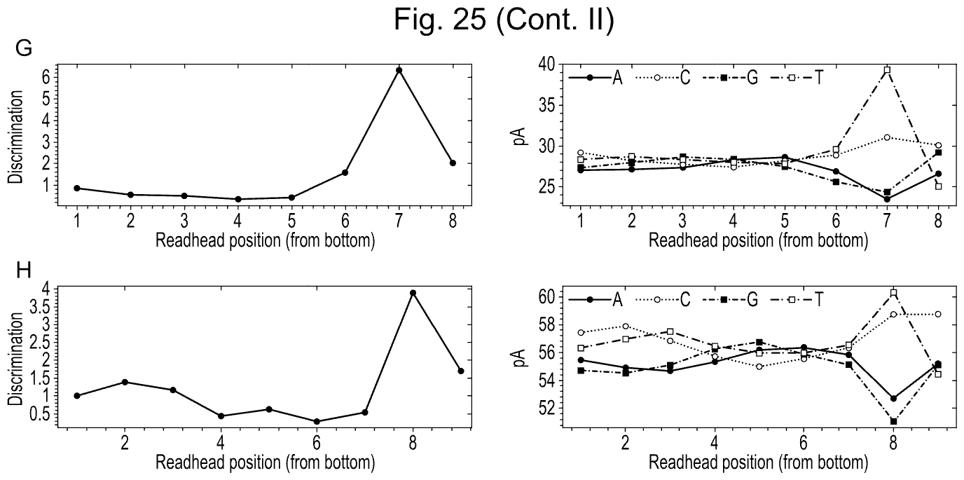

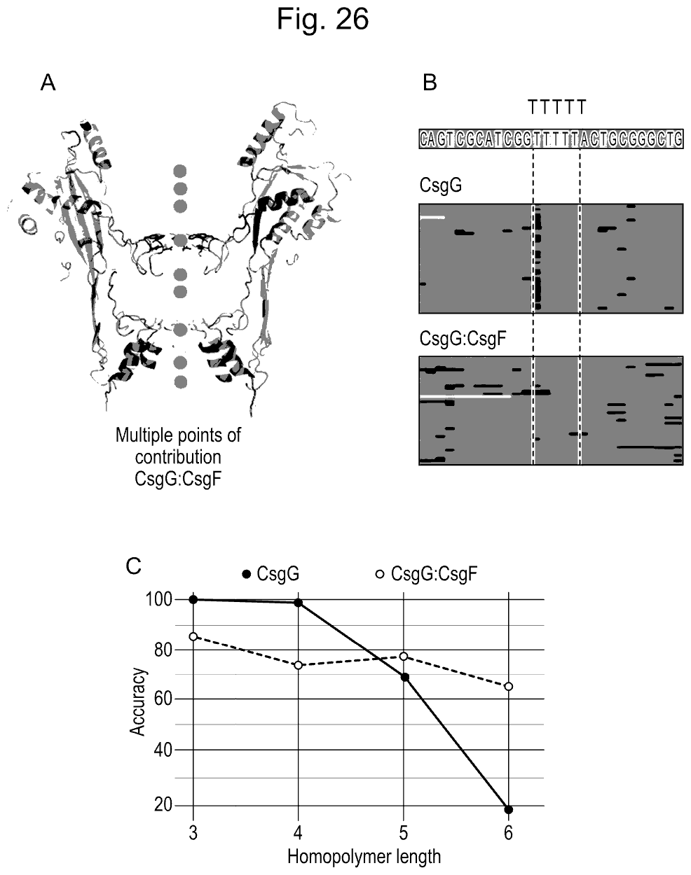

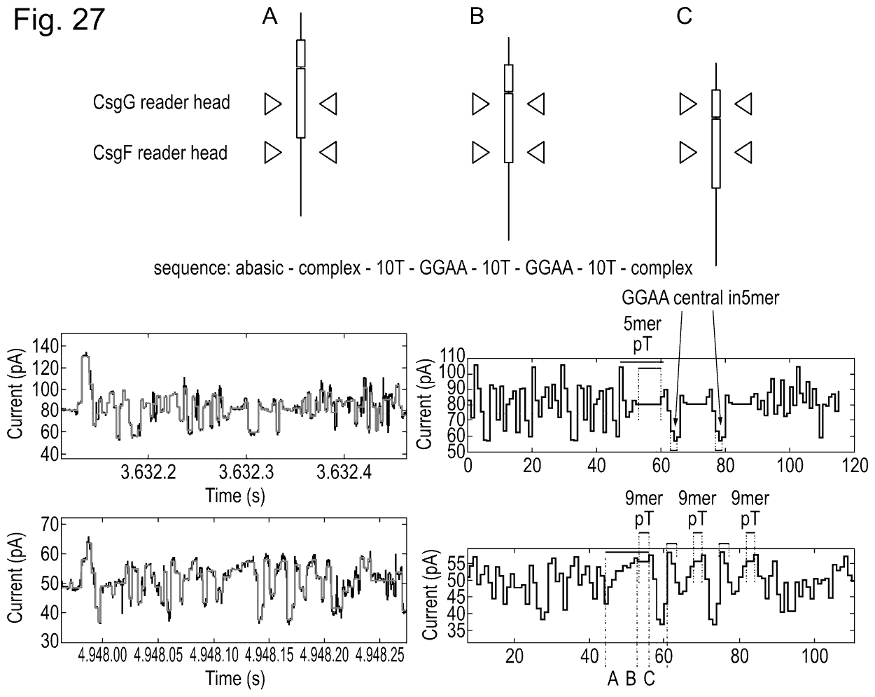

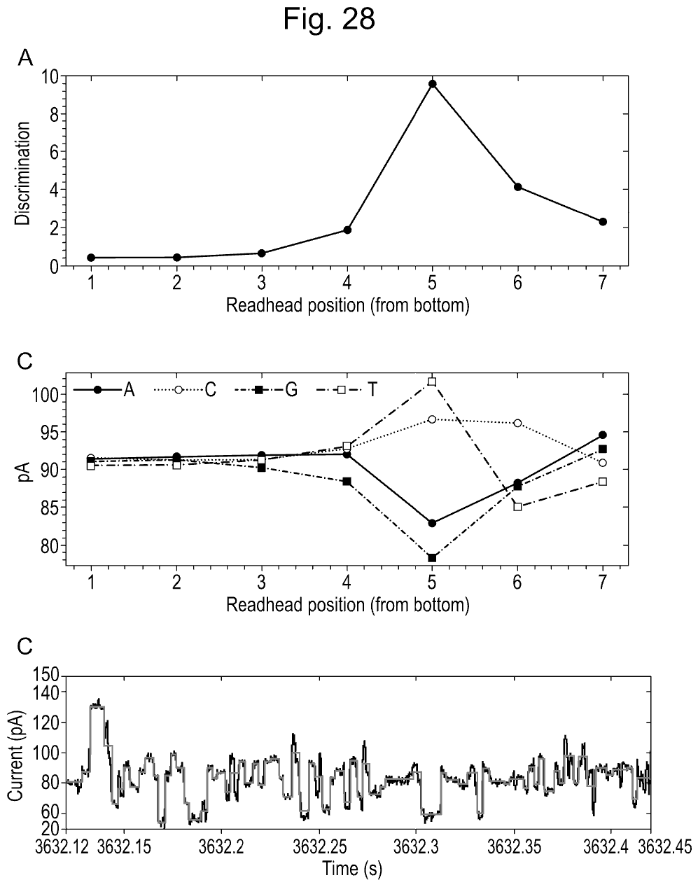

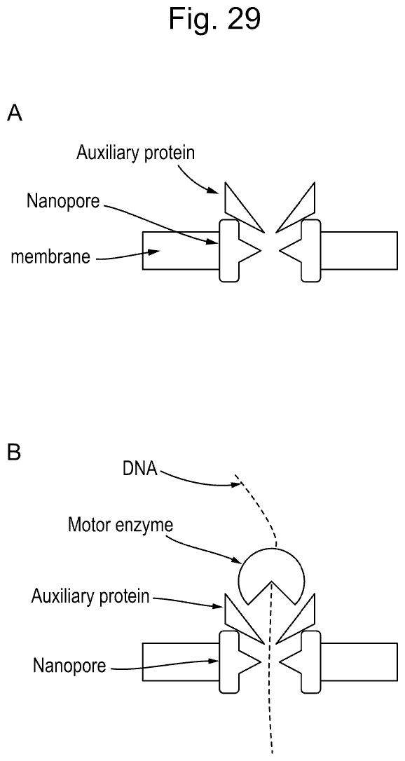

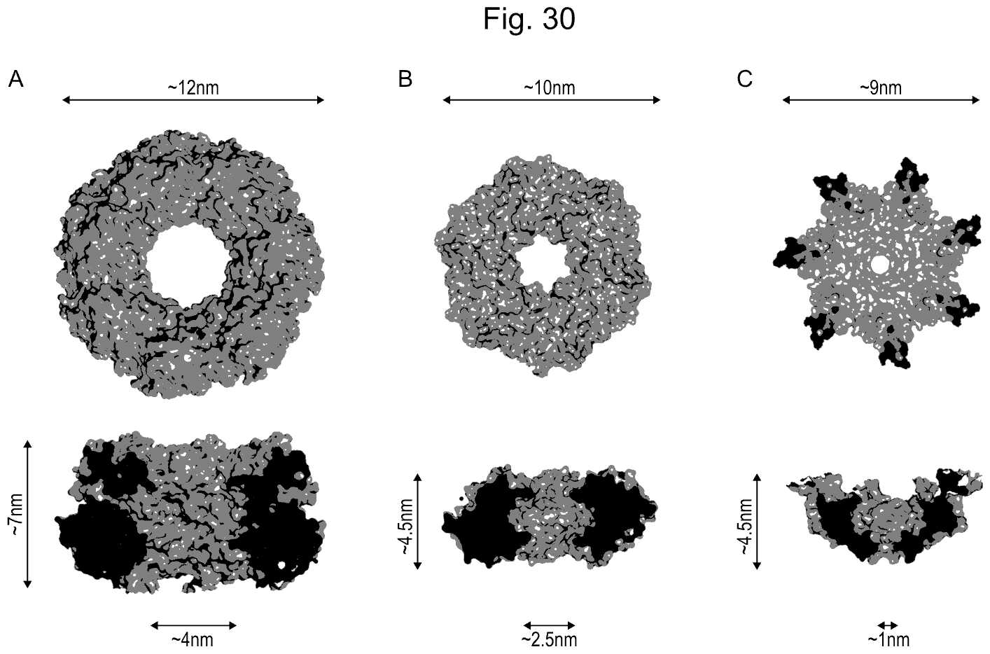

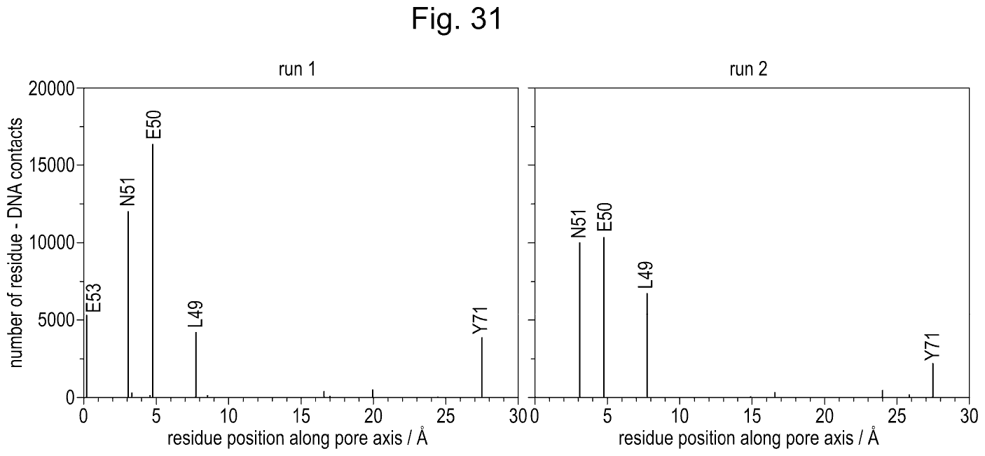

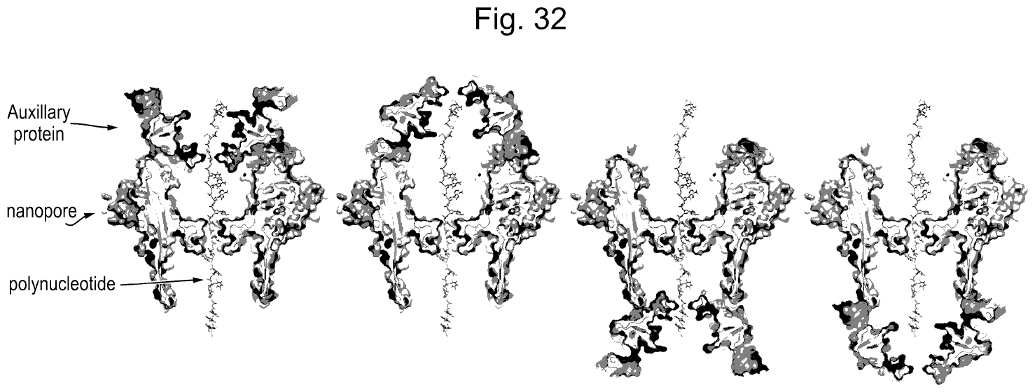

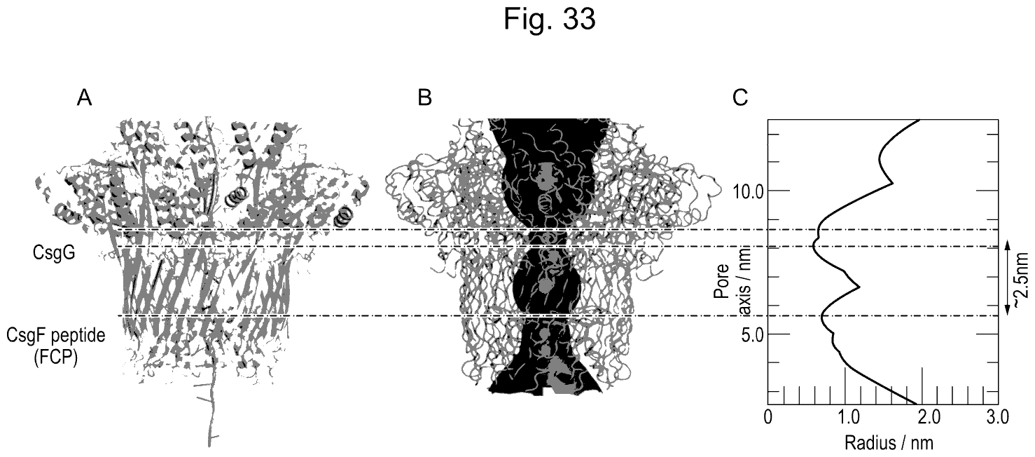

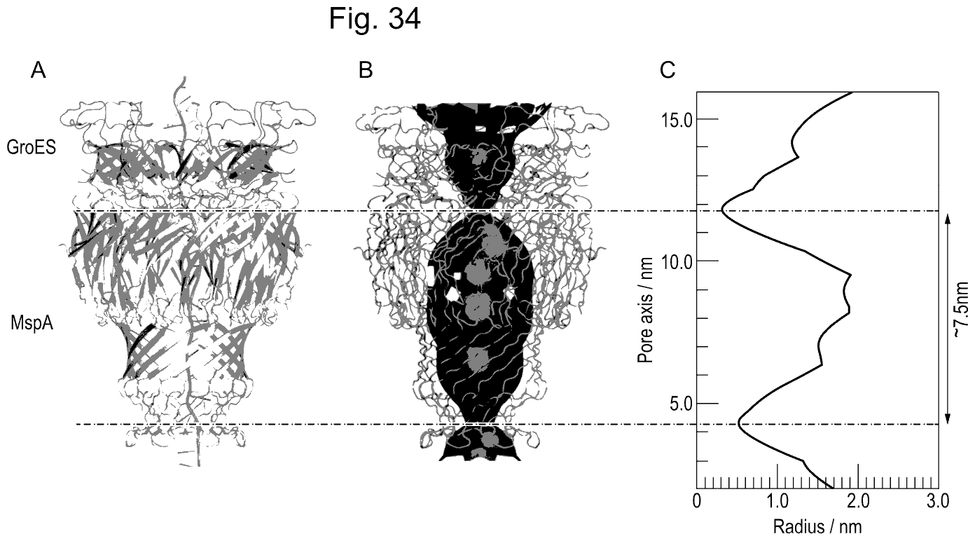

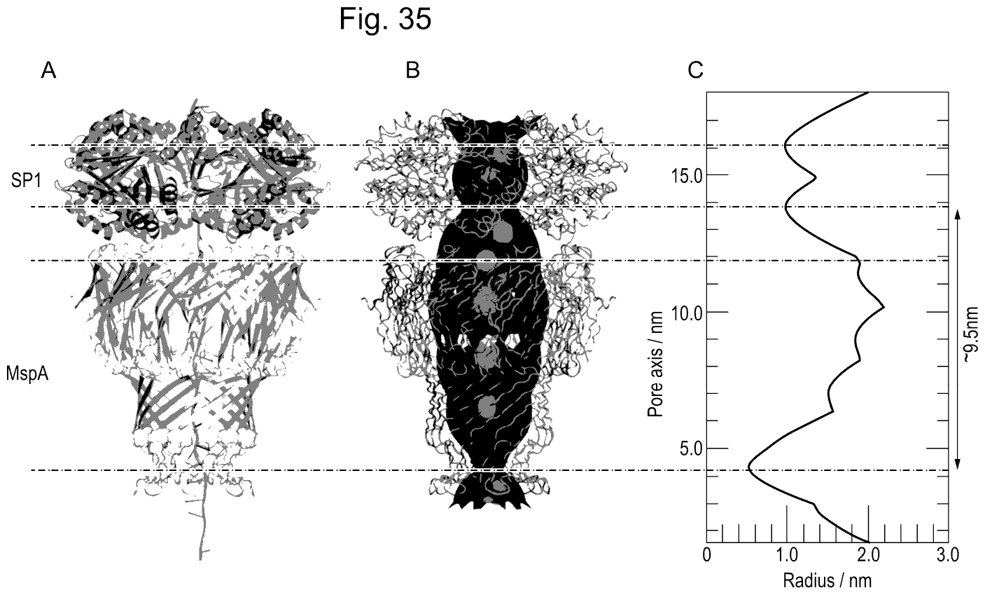

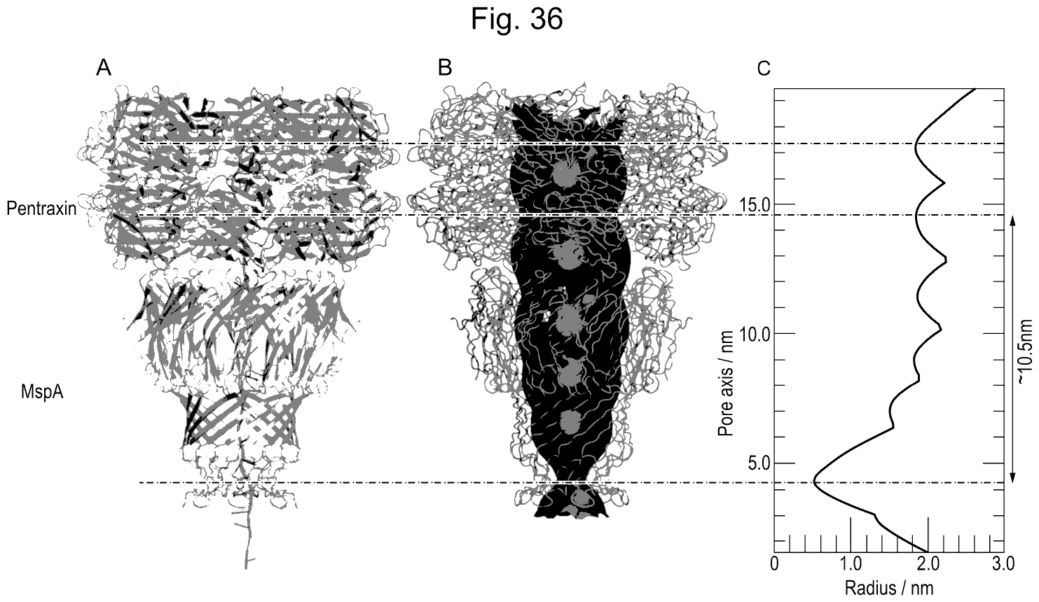

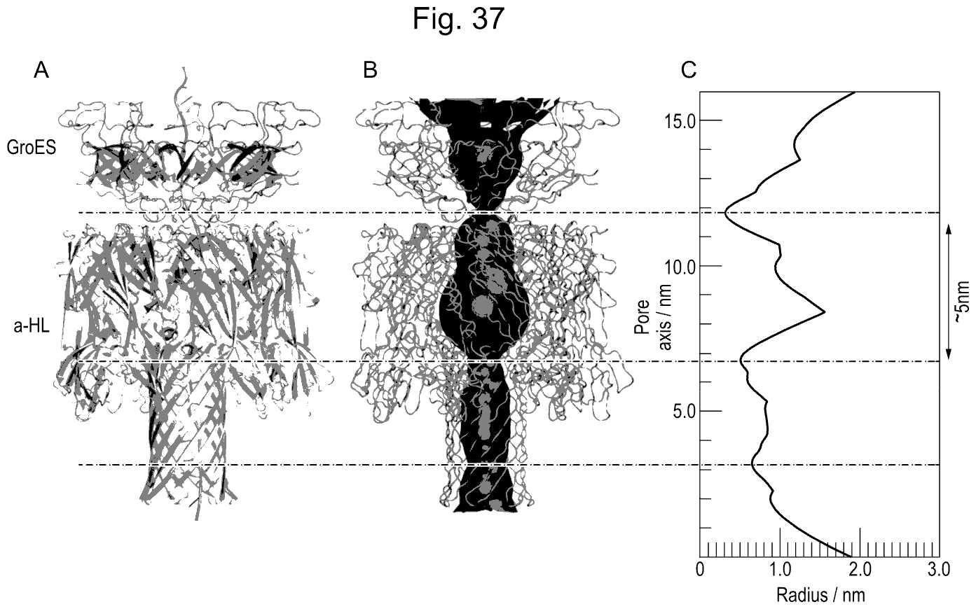

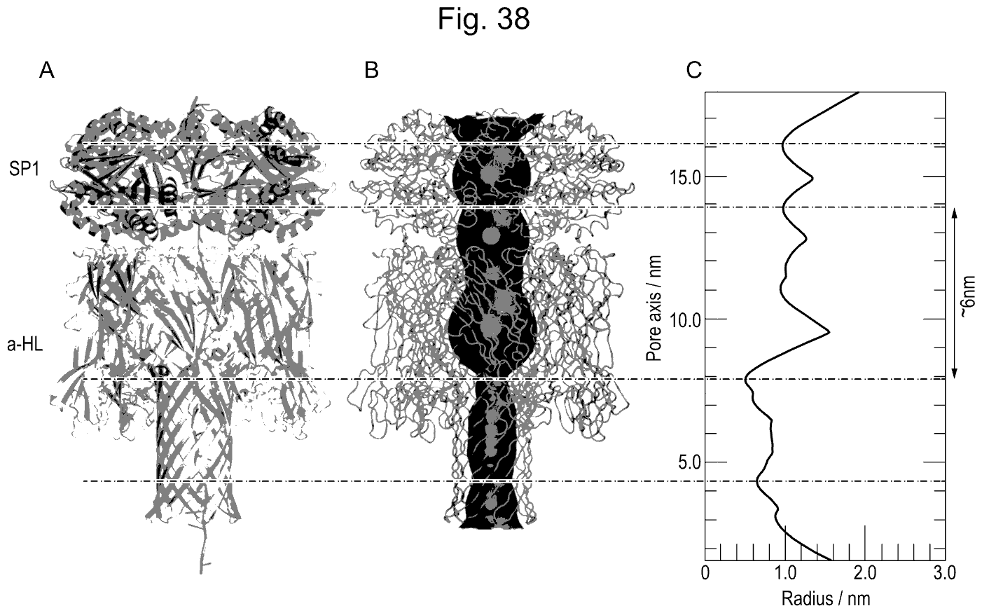

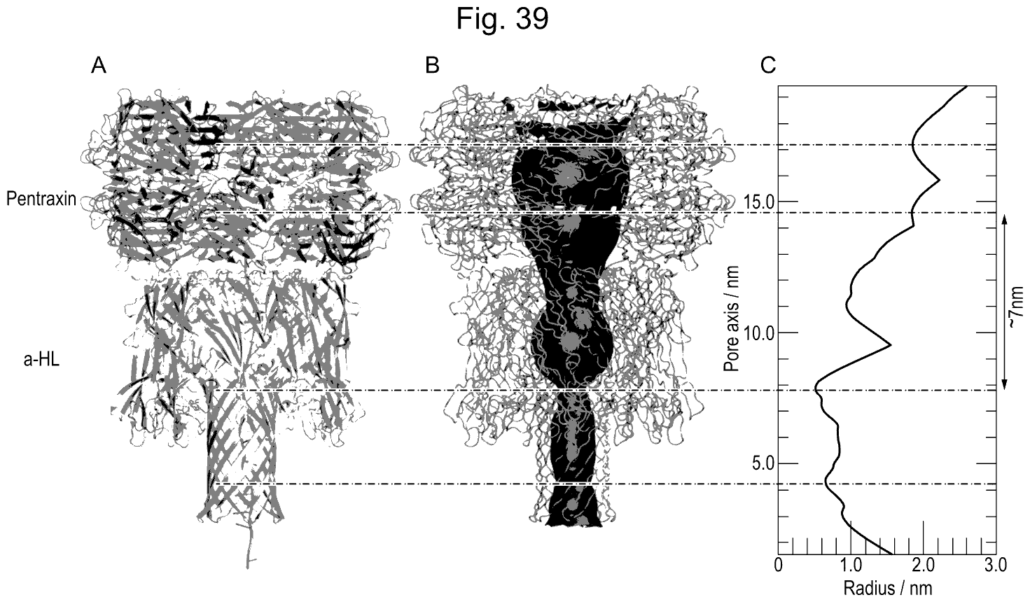

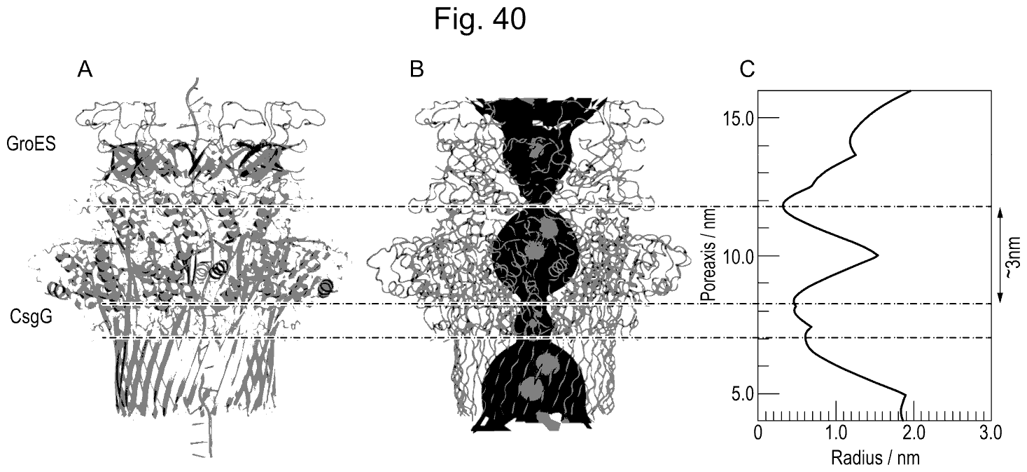

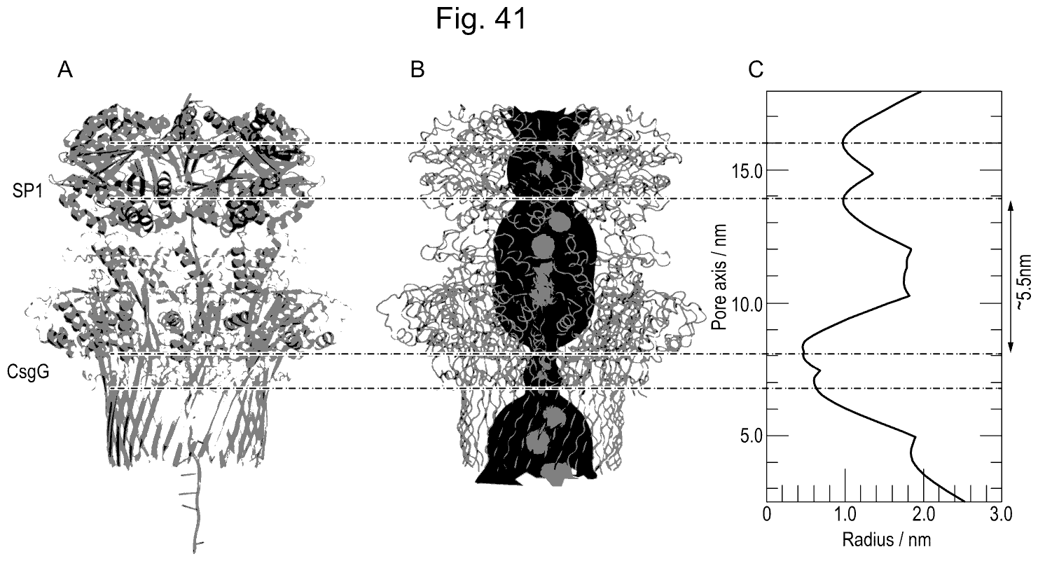

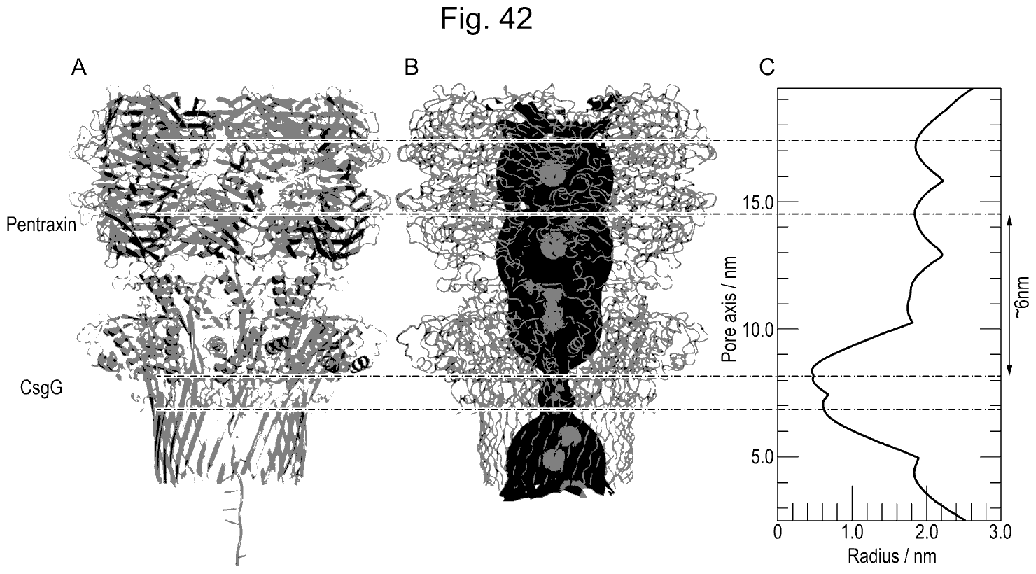

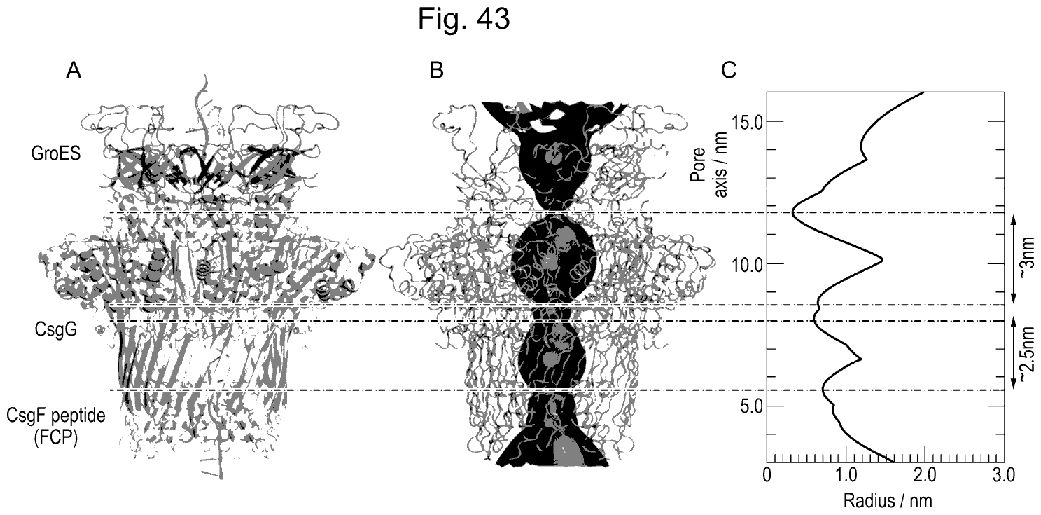

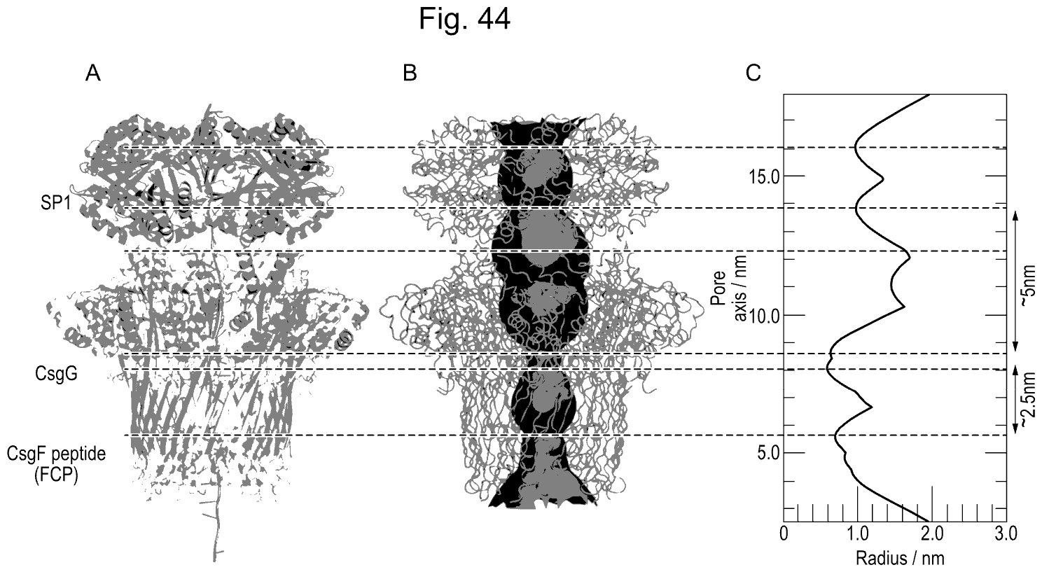

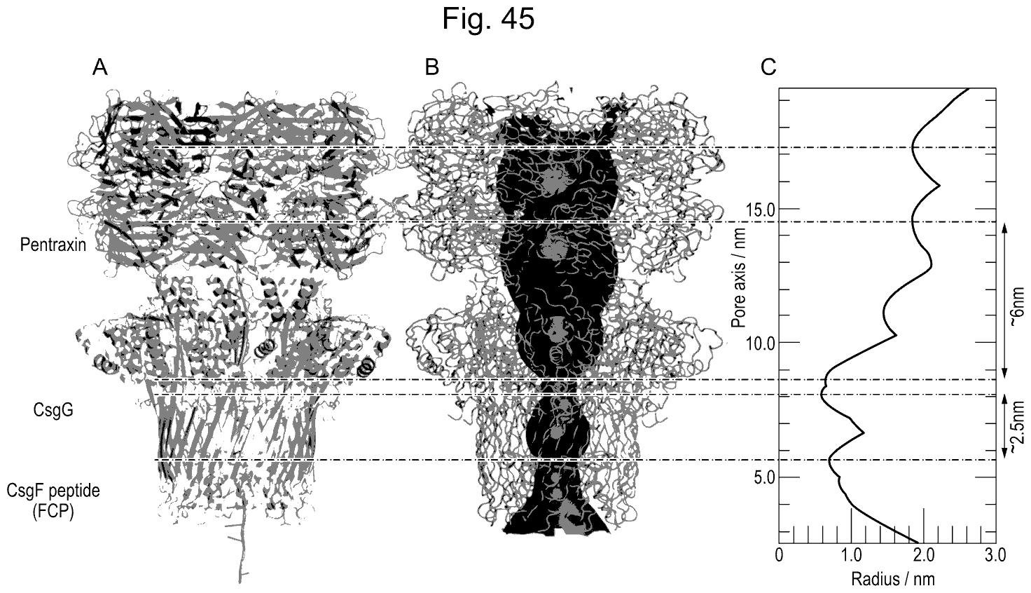

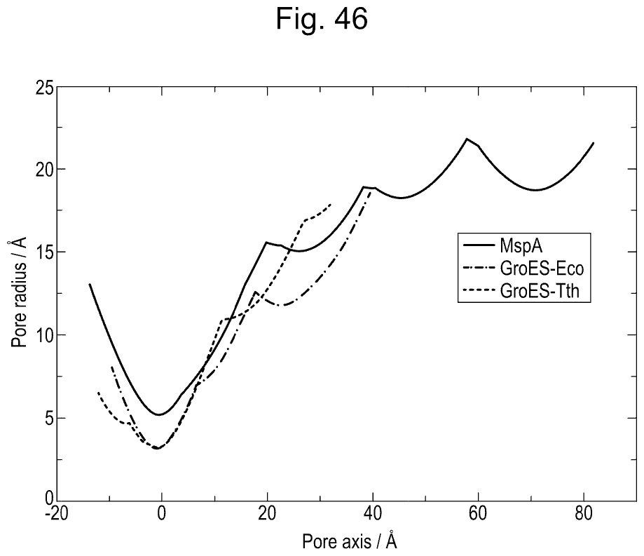

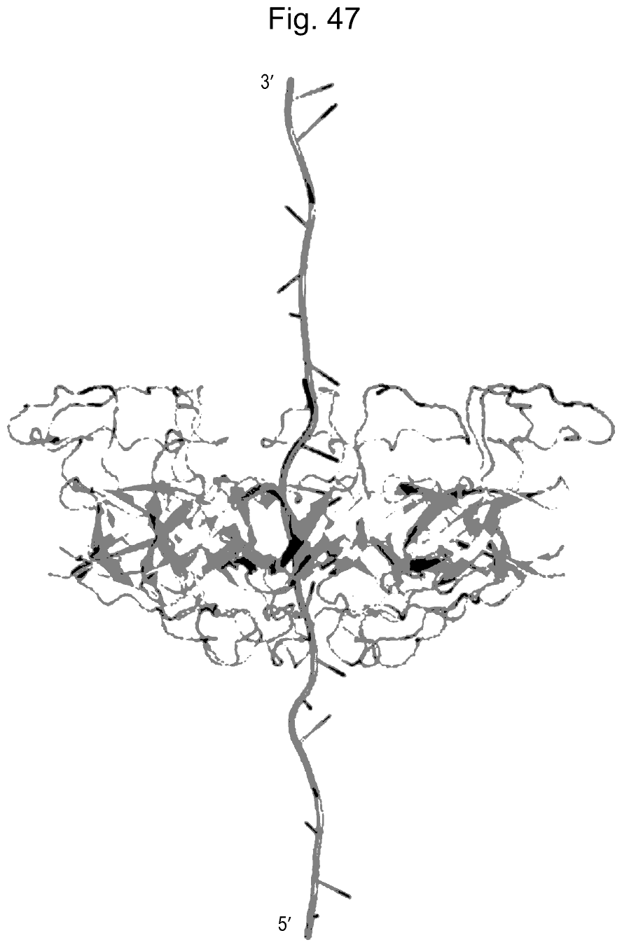

To test the hypothesis that the CsgF N-terminus corresponds to the CsgG binding region and forms the CsgF constriction peptide residing in the CsgG β-barrel lumen, Strep-tagged CsgG and His-tagged CsgF truncates were co-overexpressed in E. coli (see Methods). pNA97, pNA98, pNA99 and pNA100 encode N-terminal CsgF fragments corresponding to residues 1-27, 1-38, 1-48 and 1-64 of CsgF (SEQ ID NO:5). These peptides include the CsgF signal peptide corresponding to residues 1-19 of SEQ ID NO: 5, and thus will produce periplasmic peptides corresponding to the first 8, 19, 29 and 45 residues of mature CsgF (SEQ ID NO:6; FIG. 10A), each including a C-terminal 6× His tag. SDS-PAGE analysis of whole cell lysates revealed the presence of CsgG in all samples, as well as the presence of CsgF fragment corresponding to the first 45 residues of mature CsgF (SEQ ID NO:6; FIG. 10B). For the shorter N-terminal CsgF fragments, no detectible expression of the peptides was seen in the whole cell lysates. After two freeze/thaw cycles, cell mass of the various CsgG:CsgF fragments were further enriched by purification. Whole cell lysates as well as the eluted fractions of the Strep affinity purification were spotted onto a nitrocellulose membrane for dot blot analysis using an anti-His antibody for the detection of the His-tagged CsgF fragments (FIG. 10C). The dot blot shows the CsgF 20:64 peptide co-purifies with CsgG, demonstrating this CsgF fragment is sufficient to form a stable non-covalent complex with CsgG. For the CsgG 20:48 fragment a small amount of peptide can be seen to co-purify with CsgG, whilst no detectable levels are seen for CsgF 20:27 or CsgF 20:38 in either the whole cell lysate or the Strep affinity purification (FIG. 10C), suggesting that the latter peptides are not stably expressed in E. coli, and/or do not form a stable complex with CsgG.

### Example 5: Description of the CsgG:CsgF Interaction at Atomic Resolution

To gain an atomic level detail on the CsgG:CsgF interaction we determined the high resolution cryoEM structure of the CsgG:CsgF complex. For this purpose, CsgG and CsgF were co-expressed recombinantly in E. coli and the CsgG:CsgF complex was isolated from E. coli outer membranes by detergent extraction and purified using tandem affinity purification. Samples for electron cryo-microscopy were prepared by spotting 3 μl sample on R2/1 Holey grids (Quantifoil), coated with graphene oxide, and data was collected on a 300 kV TITAN Krios with Gatan K2 direct electron detector in counting mode. 62.000 single CsgG:CsgF particles were used to calculate a final electron density map at 3.4 Å resolution (FIG. 11A). The map allowed unambiguous docking and local rebuilding of the CsgG crystal structure, as well as the de novo building of the N-terminal 35 residues of mature CsgF (i.e. residues 20:54 of Seq ID No. 5), which encompass the FCP that binds CsgG and forms a second constriction at the height of the CsgG transmembrane β-barrel (FIG. 11C, D). The cryoEM structure shows CsgG:CsgF comprises a 9:9 stoichiometry, with C9 symmetry (FIG. 11B). The FCP binds the inside of the

CsgG β-barrel, with the C-terminus of the CsgF pointing out of the CsgG β-barrel, and the CsgF N-terminus located near the CsgG constriction. The structure shows that P35 in mature CsgF lies outside the CsgG β-barrel and forms the connection between the CsgF FCP and neck regions. The CsgF neck and head regions are not resolved in the high resolution cryoEM maps due to flexibility relative to the main body of the CsgG:CsgF complex. Three regions in the CsgG β-barrel stabilize the CsgG:CsgF interaction: (IR1) residues Y130, D155, S183, N209 and T207 in mature CsgG (SEQ ID NO: 3) form an interaction network with the N-terminal amine and residues 1 to 4 of mature CsgF (SEQ ID NO: 6), comprising four H-bonds and an electrostatic interaction; (IR2) residues Q187, D149 and E203 in mature CsgG (SEQ ID NO: 3) form an interaction network with R8 and N9 in mature CsgF (SEQ ID NO 6), encompassing three H-bonds and two electrostatic interaction; and (IR3) residues F144, F191, F193 and L199 in mature CsgG (SEQ ID NO: 3) form a hydrophobic interaction surface with residues F21, L22 and A26 in mature CsgF (SEQ ID NO: 6). The latter are located in an α-helix (helix 1) formed by residues 19-30 of mature CsgF. The conserved sequence N-P-X-F-G-G (residues 9-14 in SEQ ID NO: 6) forms an inward turn that connects the loop region formed by residues 15-19 with the CsgF helix 1. Together, these elements give rise to a constriction in the CsgG:CsgF complex, of which residue 17 (N17 in mature E. coli CsgF, SEQ ID NO: 6) forms the narrowest point, resulting in an orifice with 15 Å diameter (FIG. 11C). The second constriction (F-constriction or FC) lies approximately 15 Å and 30 Å above the top and bottom, respectively, of the constriction formed by CsgG residues 46 to 59 (G-constriction or GC).

### Example 6: Simulations to Improve CcgG-CsgF Complex Stability

Molecular dynamics simulations were performed to establish which residues in CsgG and CsgF come into close proximity. This information was used to design CsgG and CsgF mutants that could increase the stability of the complex.

Simulations were performed using the GROMACS package version 4.6.5, with the GROMOS 53a6 forcefield and the SPC water model. The cryo-EM structure of the CsgG-CsgF complex was used in the simulations. The complex was solvated and then energy minimised using the steepest descents algorithm. Throughout the simulation, restraints were applied to the backbone of the complex, however,the residue sidechains were free to move. The system was simulated in the NPT ensemble for 20 ns, using the Berendsen thermostat and Berendsen barostat to 300 K.

Contacts between CsgG and CsgF were analysed using both GROMACS analysis software and also locally written code. Two residues were defined as having made a contact if they came within 3 Angstroms. The results are shown in Table 4 below.

TABLE 4
Predicted contact frequencies of residue
pairs in the CsgG/CsgF complex:

| CsgG | CsgF | % Time spent |
|---|---|---|
| residue | residue | in contact |
| GLU 203 | ARG 8 | 88.8 |
| GLU 201 | ASN 11 | 87.4 |
| GLU 201 | PHE 12 | 84.3 |
| GLU 203 | ASN 9 | 83.6 |
| ASP 155 | GLY 1 | 81.2 |
| GLU 203 | PHE 7 | 81 |
| GLU 201 | ASN 9 | 77.2 |
| SER 183 | GLY 1 | 76.1 |
| ASN 209 | MET 3 | 70.8 |
| THR 207 | PHE 5 | 70.1 |
| ASP 149 | ARG 8 | 68.5 |
| GLN 187 | ARG 8 | 66.1 |
| ARG 142 | PHE 12 | 65.4 |
| GLU 185 | ARG 8 | 64.4 |
| ASP 149 | PHE 12 | 64.2 |
| GLN 187 | GLN 6 | 63.3 |
| GLY 205 | PHE 5 | 54 |
| GLN 197 | ASN 30 | 52.5 |
| GLN 197 | SER 31 | 51.4 |
| LYS 49 | THR 2 | 50.8 |
| PHE 144 | GLN 29 | 50.6 |
| GLU 201 | PHE 21 | 48 |
| GLN 151 | PHE 5 | 47 |
| PHE 191 | ASN 9 | 46.9 |
| ARG 142 | ASN 11 | 46.4 |
| GLN 151 | PHE 7 | 45.6 |
| TYR 196 | TYR 32 | 45.4 |
| PHE 191 | PHE 21 | 45.3 |
| PHE 193 | ALA 26 | 45.1 |
| GLU 201 | SER 25 | 44.9 |
| LEU 199 | GLN 29 | 44.7 |
| ARG 141 | PHE 12 | 43.1 |
| GLY 138 | PHE 7 | 43 |
| GLN 187 | PHE 5 | 43 |
| GLY 145 | GLN 29 | 42.4 |
| GLN 153 | GLY 1 | 42.1 |
| GLY 140 | PHE 7 | 40.5 |
| PHE 193 | TYR 32 | 39.9 |
| GLU 203 | PHE 12 | 39.7 |
| ASN 133 | PHE 5 | 35.9 |
| GLN 151 | MET 3 | 32 |
| PHE 193 | ASN 30 | 31.9 |
| SER 136 | PHE 5 | 31.7 |
| PHE 144 | SER 31 | 30.3 |
| TYR 130 | GLY 1 | 30 |
| GLN 187 | PHE 7 | 29.9 |
| PHE 192 | ASN 30 | 28.9 |
| GLY 138 | PHE 5 | 28.3 |
| ILE 194 | TYR 32 | 26.7 |
| ASN 209 | GLY 1 | 26.1 |
| PHE 192 | GLN 29 | 25.8 |
| PHE 193 | GLN 29 | 25.4 |
| PHE 193 | GLN 27 | 23.9 |
| ASP 149 | GLY 13 | 22.7 |
| TYR 196 | ASN 30 | 22.6 |
| PHE 192 | SER 31 | 22.2 |
| ASP 148 | PHE 12 | 22 |
| GLY 140 | PHE 12 | 21.7 |
| TYR 196 | ASP 34 | 21.6 |
| ARG 198 | SER 31 | 19.9 |
| VAL 139 | PHE 7 | 19.5 |
| PHE 191 | ALA 26 | 18.3 |
| ASN 132 | GLY 1 | 18.1 |
| TYR 195 | TYR 32 | 17.9 |
| GLN 197 | ALA 28 | 17.6 |
| GLN 151 | ARG 8 | 16.9 |
| PHE 191 | LEU 22 | 16.5 |
| PHE 191 | GLN 29 | 15.6 |
| THR 206 | PHE 5 | 14.7 |
| GLN 153 | MET 3 | 14.3 |
| PHE 192 | TYR 32 | 13.8 |
| GLU 201 | GLN 29 | 13.3 |
| ARG 142 | SER 25 | 13.3 |
| PHE 144 | ASN 30 | 12.6 |
| ARG 142 | ARG 8 | 12.6 |
| PHE 191 | ASN 11 | 12.3 |
| GLU 131 | THR 2 | 12.2 |
| ASN 133 | GLY 1 | 11.3 |
| GLY 205 | PHE 7 | 11.2 |
| GLN 151 | PHE 12 | 10.4 |
| ASN 132 | PHE 5 | 10.3 |
| GLU 202 | PHE 12 | 10.2 |
| ASP 149 | PHE 7 | 10.2 |

### Materials and Methods for Structural Determination of the CsgG:CsgF Complex:

### Cloning

For the expression of E. coli CsgG as outer membrane localized pore, the coding sequence of E. coli CsgG (SEQ ID NO:1) was cloned into pASK-Iba12, resulting in plasmid pPG1 (Goyal et al. 2013).

For the expression of C-terminally 6×-His tagged CsgF in the E. coli cytoplasm, the coding sequence for mature E. coli CsgF (SEQ ID NO:6; i.e. CsgF without its signal sequence) was cloned into pET22b via the Ndel and EcoRI sites, using a PCR product generated using the primers “CsgF-His_pET22b_FW” (SEQ ID NO:46) and “CsgF-His_pET22b_Rev” (SEQ ID NO:47), resulting in the CsgF-His expression plasmid pNA101.

The pNA62 plasmid, a pTrc99a based vector expressing csgF-His and csgG-strep, was created based on pGV5403 (pTrc99a with the pDEST14 Gateway® cassette integrated). The pGV5403 ampicillin resistance cassette was replaced by a streptomycin/spectinomycin resistance cassette. A PCR fragment encompassing part of the E. coli MC4100 csgDEFG operon corresponding to the coding sequences of csgE, csgF and csgG was generated with primers csgEFG_pDONR221_FW (SEQ ID NO:48) and csgEFG_pDONR221_Rev (SEQ ID NO:49), and inserted in pDONR221 (ThermoFisher Scientific) via BP Gateway® recombination. Next, this recombinant csgEFG operon from the pDONR221 donor plasmid was inserted via LR Gateway® recombination into pGV5403 with streptomycin/spectinomycin resistance cassette. Via PCR, a 6× His-tag was added to the CsgF C-terminus using primers Mut_csgF_His_FW (SEQ ID NO:50) and Mut_csgF_His_Rev (SEQ ID NO:51). Finally, csgE was removed by outwards PCR (primers DelCsgE_FW (SEQ ID NO:52) and DelCsgE_Rev (SEQ ID NO:53)) to obtain pNA62.

Constructs for the periplasmic expression of C-terminally His-tagged CsgF fragments corresponding to the putative constriction peptides (FIG. 10 A) were created by outwards PCR on pNA62, a pTrc99a based vector expressing CsgF-his and CsgG-strep. Primer combinations were as follows: pNa62_CsgF_histag_Fw (SEQ ID NO:45) as forward primers, with CsgF_d27_end (SEQ ID NO:41), CsgF_d38_end (SEQ ID NO:42), CsgF_d48_end (SEQ ID NO:43) or CsgF_d64_end (SEQ ID NO:44) as reverse primers to create pNA97, pNA98, pNA99 and pNA100 respectively.

In pNA97 csgF is truncated to SEQ ID NO:7, encoding a CsgF fragment including residues 1-27 (SEQ ID NO:8); In pNA98 csgF is truncated to SEQ ID NO:9, encoding a CsgF fragment including residues 1-38 (SEQ ID NO:10); In pNA99 csgF is truncated to SEQ ID NO:11, encoding a CsgF fragment including residues 1-48 (SEQ ID NO:12); and in pNA100 csgF is truncated to SEQ ID NO:13, encoding a CsgF fragment including residues 1-64 (SEQ ID

NO:14). Expression of pNA97, pNA98, pNA99 and pNA100 in E. coli does result in production of the CsgG pore (SEQ ID NO:3) in the outer membrane, as well as periplasmic targeting of CsgF-derived peptides with sequences:

(SEQ ID NO: 37 + 6xHis)
“GTMTFQFRHHHHHH”,
(SEQ ID NO: 38 + 6xHis)
“GTMTFQFRNPNFGGNPNNGHHHHHH”,
(SEQ ID NO: 39 + 6xHis)
“GTMTFQFRNPNFGGNPNNGAFLLNSAQAQHHHHHH”,
and
(SEQ ID NO: 40 + 6xHis)
“GTMTFQFRNPNFGGNPNNGAFLLNSAQAQNSYKDPSYNDDFGIETHHHH
HH”,

respectively.

### Strains

E. coli Top10 (F⁻mcrA Δ(mrr⁻hsdRMS⁻mcrBC) Φ80lacZΔM15 ΔlacX74 recA1 araD139 Δ(araleu) 7697 galU galK rpsL (StrR) endA1 nupG) was used for all cloning procedures. E. coli C43(DE3) (F⁻ompT hsdSB (rB⁻mB⁻) gal dcm (DE3)) and Top10 were used for protein production.

### Recombinant CsgG:CsgF Complex Production via Co-Expression

For co-expression of E. coli CsgF (SEQ ID NO:5) and CsgG (SEQ ID NO:2), both recombinant genes including their native Shine Dalgarno sequences were placed under control of the inducible trc promotor in a pTrc99a-derived plasmid to form plasmid pNA62. CsgG and CsgF were overexpressed in E. coli C43(DE3) cells transformed with plasmid pNA62 and grown at 37° C. in Terrific Broth medium. When the cell culture reached an optical density (OD) at 600 nm of 0.7, recombinant protein expression was induced with 0.5 mM IPTG and left to grow for 15 hours at 28° C., before being harvested by centrifugation at 5500 g.

Recombinant CsgG:CsgF Complex Production via In Vitro Reconstitution

Full-length E. coli CsgG (SEQ ID NO:2) modified with a C-terminal StrepII-tag was overexpressed in E. coli BL21 (DE3) cells transformed with plasmid pPG1 (Goyal et al. 2013). The cells were grown at 37° C. to an OD 600 nm of 0.6 in Terrific Broth medium. Recombinant protein production was induced with 0.0002% anhydrotetracyclin (Sigma) and the cells were grown at 25° C. for a further 16 h before being harvested by centrifugation at 5500 g.

E. coli CsgF (SEQ ID NO:6; i.e. lacking the CsgF signal sequence) in a C-terminal fusion with a 6× His-tag was overexpressed in the cytoplasm of E. coli BL21(DE3) cells transformed with plasmid pNA101. Cells were grown at 37° C. to an OD of 600 nm followed by induction by 1 mM IPTG and left to express protein 15h at 37° C. before being harvested by centrifugation at 5500 g.

### Recombinant Protein Purification of the CsgG:CsgF Complex, CsgG, and CsgF

E. coli cells transformed with pNA62 and co-expressing CsgG-Strep and CsgF-His were resuspended in 50 mM Tris-HCl pH 8.0, 200 mM NaCl, 1 mM EDTA, 5 mM MgCl₂, 0.4 mM AEBSF, 1 μg/mL Leupeptin, 0.5 mg/mL DNase I and 0.1 mg/mL lysozyme. The cells were disrupted at 20 kPsi using a TS series cell disruptor (Constant Systems Ltd) and the lysed cell suspension incubated 30′ with 1% n-dodecyl-β-d-maltopyranoside (DDM; Inalco) for further cell lysis and extraction of outer membrane components. Next, remaining cell debris and membranes were spun down by ultracentrifugation at 100.000 g for 40′. Supernatant was loaded onto a 5 mL HisTrap column equilibrated in buffer A (25 mM Tris pH8, 200 mM NaCl, 10 mM imidazole, 10% sucrose and 0.06% DDM). Column was washed with >10 CVs 5% buffer B (25 mM Tris pH8, 200 mM NaCl, 500 mM imidazole, 10% sucrose and 0.06% DDM) ion buffer A and eluted with a gradient of 5-100% buffer B over 60 mL.

Eluent was diluted 2-fold before loading overnight on a 5 mL Strep-tactin column (IBA GmbH) equilibrated with buffer C (25 mM Tris pH8, 200 mM NaCl, 10% sucrose and 0.06% DDM). Column was washed with >10 CVs buffer C and protein was eluted by the addition of 2.5 mM desthiobiotin. Next 500 μL of the peak fraction of the double-affinity purified complex was injected on a Superose 6 10/30 (GE Healthcare) equilibrated with Buffer D (25 mM Tris pH8, 200 mM NaCl and 0.03% DDM), and run at 0.5 mL/min to prepare samples for electron microscopy. Protein concentration was determined based on calculated absorbance at 280 nm and assuming 1/1 stoichiometry. Buffer D (25 mM Tris pH8, 200 mM NaCl and 0.03% DDM)

CsgG-strep purification for in vitro reconstitution is identical to the protocol for CsgG:CsgF when omitting sucrose in the buffers and bypassing the IMAC and size exclusion steps.

CsgF-His purification for in vitro reconstitution was performed by resuspension of the cell mass in 50 mM Tris-HCl pH 8.0, 200 mM NaCl, 1 mM EDTA, 5 mM MgCl2, 0.4 mM AEBSF, 1 μg/mL Leupeptin, 0.5 mg/mL DNase I and 0.1 mg/mL lysozyme. The cells were disrupted at 20 kPsi using a TS series cell disruptor (Constant Systems Ltd) and the lysed cell suspension was centrifuged at 10.000 g for 30 min to remove intact cells and cell debris. Supernatant was added to 5 mL Ni-IMAC-beads (Workbeads 40 IDA, Bio-Works Technologies AB) equilibrated with buffer A (25 mM Tris pH8, 200 mM NaCl, 10 mM imidazole) and left incubating for 1 hour at 4° C. Ni-NTA beads were pooled in a gravity flow column and washed with 100 mL of 5% buffer B (25 mM Tris pH8, 200 mM NaCl, 500 mM imidazole diluted in buffer A. Bound protein was eluted by stepwise increase of Buffer B (10% steps of each 5 mL).

### In Vitro Reconstitution of the CsgG:CsgF Complex

Purified CsgG and CsgF were pooled and used to in vitro reconstitute the complex. Therefore a molar ratio of 1 CsgG:2 CsgF was mixed to saturate the CsgG barrel with CsgF. Next, the reconstituted mixture was injected on a Superose 6 10/30 column (GE Healthcare) equilibrated with Buffer D (25 mM Tris pH8, 200 mM NaCl and 0.03% DDM), and run at 0.5 mL/min to prepare samples for electron microscopy. Protein concentration was determined based on calculated absorbance at 280 nm and assuming 1/1 stoichiometry.

### Structural Analysis Using Electron Microscopy

Sample behavior of the size exclusion fraction is probed using negative stain electron microscopy. Samples are stained with 1% uranyl formate and imaged using an in-house 120 kV JEM 1400 (JEOL) microscope equipped with a LaB6 filament. Samples for electron cryomicroscopy were prepared by spotting 2 μL sample onto R2/1 continuous carbon (2 nm) coated grids (Quantifoil), manually blotted and plunged in liquid ethane using an in house plunging device. Sample quality was screened on the in-house JEOL JEM 1400 before collecting a dataset on a 200 kV TALOS ARCTICA (FEI) microscope equipped with a Falcon-3 direct electron detection camera. Images were motion corrected with MotionCor2.1 (Zheng et al. 2017), defocus values were determined using ctffind4 (Rohou and Grigorieff, 2015) and data was further analysed using a combination of RELION (Scheres, 2012) and EMAN2 (Ludtke, 2016). C9 Symmetry was imposed during 3D model generation and refinement on selected 2D class averages featuring additional density for a head group.

For high resolution cryoEM analysis, CsgG:CsgF samples were prepared for electron cryo-microscopy by spotting 3 μl sample on R2/1 Holey grids (Quantifoil), coated with graphene oxide (Sigma Aldrich), manually blotted and plunged in liquid ethane using CP3 plunger (Gatan). Sample quality was screened on the in-house JEOL JEM 1400 before collecting a dataset on a 300kV TITAN KRIOS (FEI, Thermo-Scientific) microscope equipped K2 Summit direct electron detector (Gatan). The detector was used in counting mode with a cumulative electron dose of 56 electrons per Å² spread over 50 frames. 2045 images were collected with a pixel size of 1.07 Å. Images were motion-corrected with MotionCor2.1 (Zheng et al. 2017) and defocus values were determined using ctffind4 (Rohou and Grigorieff, 2015). Particles were picked automatically using Gautomatch (Dr. Kai Zhang) and data was further analysed using a combination of RELION2.0 (Kimanius et al. 2016, Elife 5. pii: e18722) and EMAN2 (Ludtke, 2016). C9 Symmetry was imposed during 3D model generation and refinement on selected 2D class averages featuring additional density for the head group corresponding to CsgF. 62.000 particles were used to calculate the final map at 3.4 Å resolution. De novo model building of CsgF was done with COOT (Brown et al. 2015 Acta Crystallogr D Biol Crystallogr 71(Pt 1):136-53) and iterative cycles of model building and refinement of the full complex was done with PHENIX (Afonine 2018, Acta Crystallogr D Struct Biol 74(Pt 6):531-544) real-space refinement in combination with COOT.

### Protein Expression and Purification of CsgG:CsgF Fragments

CsgF fragments and CsgG were co-expressed, with CsgF fragments being C-terminally His-tagged and CsgG fused C-terminally to a Strep tag. The CsgG:CsgF fragments complex was over-expressed in E. coli Top10 cells, transformed with plasmid pNA97, pNA98, pNA99 or pNA100. Plates were grown at 37° C. ON, and a colony was resuspended in LB medium supplemented with Streptomycin/spectomycin. When the cell cultures reached an optical density (OD) at 600 nm of 0.7, recombinant protein expression was induced with 0.5 mM IPTG and left to grow for 15 hours at 28° C., before being harvested by centrifugation at 5500 g. Pellets were frozen at −20° C.

Cell mass for the various CsgG:CsgF fragment co-expressions was resuspended in 200 mL 50 mM Tris-HCl pH 8.0, 200 mM NaCl, 1 mM EDTA, 5 mM MgCl₂, 0.4 mM AEBSF, 1 μg/mL Leupeptin, 0.5 mg/mL DNase I and 0.1 mg/mL lysozyme, sonicated and incubated with 1% n-dodecyl-β-d-maltopyranoside (DDM; Inalco) for further cell lysis and extraction of outer membrane components. Next, remaining cell debris and membranes were spun down by centrifugation at 15.000 g for 40′. The supernatant was incubated with 100 μL Strep-tactin beads at RT for 30 min. Strep beads were washed with buffer (25 mM Tris pH8, 200 mM NaCl, and 1% DDM) by centrifugation and bound proteins were eluted by the addition of 2.5 mM desthiobiotin in 25 mM Tris pH8, 200 mM NaCl, 0.01% DDM.

### Production of CsgG:FCP by In Vitro Reconstitution.

A synthetic peptide corresponding to the N-terminal 34 residues of mature CsgF (SEQ ID NO: 6) was diluted to 1 mg/ml in buffer 0.1 M MES, 0.5 M NaCl, 0.4 mg/ml EDC (1-ethyl-3-(3-dimethylaminopropyl)carbodiimide), 0.6 mg/ml NHS (N-hydroxysuccinimide) and incubated for 15 min at room temperature to allow activation of the peptide carboxyterminus. Next, 1 mg/ml Cadaverin-Alexa594 in PBS was added during a 2 h incubation to allow covalent coupling at room temperature. The reaction was quenched via buffer exchange to 50 mM Tris, NaCl, 1 mM EDTA, 0.1% DDM using Zeba Spin filters.

Labelled peptide was added to strep-affinity purified CsgG in 50 mM Tris, 100 mM NaCl, 1 mM EDTA, 5 mM LDAO/C8D4 in a 2:1 molar ratio during 15 minutes at room temperature to allow reconstitution of the CsgG:FCP complex. After pull down of CsgG-strep on StrepTactin beads, the sample was analysed on native-PAGE.

### Example 7: Further Stabilization of CsgG:CsgF Complex by Covalent Cross Linking

Although full length and some of the truncated versions of CsgF make stable CsgG:CsgF complexes with the CsgG pore, CsgF can still be dislodged from the barrel region of CsgG pore under certain conditions. Therefore, it is desirable to make a covalent link between the CsgG and CsgF subunits. Based on molecular simulation studies, positions of CsgG and CsgF that are in close proximity to each other have been identified (Example 6 and Table 4). Some of these identified positions have been modified to incorporate a Cysteine in both CsgG and CsgF. FIG. 16 shows an example of thiol-thiol bond formation between Q153 position of CsgG and G1 position of CsgF. CsgG pore containing Q153C mutation was reconstituted with CsgF containing G1C mutation and incubated for 1 hour enabling S—S bond formation. When the complex is heated to 100° C. in the absence of DTT, a 45kDa band corresponding to dimer between CsgG monomer and CsgF monomer (CsgGm-CsgFm) can be seen indicating the S—S bond formation between the two monomers (CsgGm is 30kDa and CsgFm is 15kDa) (FIG. 16.A). This band disappears when the heating is done in the presence of DTT. DTT breaks down the S—S bond. When the CsgG:CsgF complex incubated overnight instead of 1 hour, the extend of CsgGm-CsgFm dimer formation increases (FIG. 16.A). Mass spectroscopy methods have been carried out to further identify the dimer band. Gel purified protein was proteolytically cleaved to generate tryptic peptides. LC-MS/MS sequencing methods were performed, resulting in the identification of S—S bond between the Q153 position of CsgG and G1 position of CsgF (FIG. 16.B). Oxidising agents such as copper-orthophenanthroline can be used to enhance the S—S bond formation. When CsgG pore containing N133C modification is reconstituted with CsgF containing T4C modification in the presence of copper-orthophenanthroline as described in methods section and then broken down to its constituent monomers by heating to 100° C. in the absence of DTT, a strong dimer band corresponding to CsgGm-CsgFm can be observed on SDS-PAGE (FIG. 17, lanes 3 and 4). When the heating was carried out in the presence of DTT, the dimer breaks down to its constituent monomers (FIG. 17, lanes 1 and 2).

### Example 8: Electrophysiological Characterisation of CsgG:CsgF Complexes

The signal observed when a DNA strand translocates through CsgG is well characterised when the pore is inserted in the copolymer membrane and experiments are carried out using the MinION of Oxford Nanopore Technologies (FIG. 28). Y51, N55 and F56 of each subunit of CsgG form the constriction of the CsgG pore (FIG. 12). This sharp constriction serves as the reader head of the CsgG pore (FIG. 28A) and is able to accurately discriminate a mixed sequence of A,C,G and T as it passes through the pore. This is because the measured signal contains characteristic current deflections from which the identity of the sequence can be derived. However, in homopolymeric regions of DNA, the measured signal may not show current deflections of sufficient magnitude to allow single base identification; such that an accurate determination of the length of a homopolymer cannot be made from the magnitude of the measured signal alone (FIGS. 23B and C). The reduction in accuracy of the CsgG reader head is correlated to the length of the homopolymeric region (FIG. 26C).

When CsgF interacts with the CsgG pore to make the CsgG:CsgF complex, CsgF introduces a second reader head within the CsgG barrel. This second reader head primarily consists of the N17 position of Seq. ID No. 6. A static strand experiment as described in the methods section and FIG. 24 was carried out to map the two reader heads of the CsgG:CsgF complex experimentally, and results indicate the presence of the two reader heads that are separated from each other by approximately 5-6 bases (FIGS. 24, B, C and D). Reader head discrimination plot for the CsgG:CsgF complex shows that the second reader head introduced by CsgF contributes less to the base discrimination than that of the CsgG reader head (FIG. 24A). Surprisingly, when a second reader head is introduced by CsgF within the CsgG barrel, the homopolymeric region which was flat previously shows a step wise signal (FIGS. 27B and C). These steps contain information that can be used to identify the sequence accurately resulting in a decrease in errors. Accuracy of the DNA signal of the CsgG:CsgF complex remains relatively constant over a longer homopolymeric length compared to the accuracy profile of the CsgG pore by itself (FIG. 26C).

CsgG:CsgF complexes made in any of the methods described in the methods section can be used to characterise the complex in DNA sequencing experiments. Signals of a lambda DNA strand passing through various CsgG:CsgF complexes made by different methods consisting of different CsgG mutant pores and different CsgF peptides with different lengths are shown in FIGS. 18-21. Reader head discrimination of those pore complexes and their base contribution profiles are shown in FIG. 25 (A-H). Surprisingly, different modifications at constrictions of both CsgG pore and the CsgF peptide can alter the signal of the CsgG:CsgF pore complex significantly. For example, when the CsgG:CsgF complexes are made with the same CsgG pore, but with two different CsgF peptides of the same length containing either Asn or Ser at position 17 (of Seq ID No. 6) (made by the same method of co-expression of the full length CsgF protein followed by TEV protease cleavage of CsgF between positions 35 and 36), the signals generated are different from each other (FIG. 18). The CsgG:CsgF complex with Ser at position 17 of the CsgF peptide shows lower noise and higher signal:noise ratio compared to the CsgG:CsgF complex with Asn at position 17 of the CsgF peptide. Similarly, when the same CsgG pore was reconstituted with two different peptides of CsgF of the same length (1-35 of Seq ID No. 6) but with either Ser or Val at positon 17 to make the CsgG:CsgF complexes, the complex with Val at position 17 of CsgF shows a noisier signal than the complex with Ser at position 17 of CsgF (FIG. 19). When the same CsgF peptide of the same length was reconstituted with different CsgG pores containing different mutations at the CsgG reader head (positions 51, 55 and 56), the resulting CsgG:CsgF complexes showed very different signals (FIG. 20, A-F) with different signal to noise ratios (FIG. 22). Surprisingly, when different lengths of CsgF peptides that contained the same constriction region were reconstituted with the same CsgG pore to make CsgG:CsgF complexes, they gave signals with a different range (FIG. 21). CsgG:CsgF complex which contains the shortest CsgF peptide (1-29 of Seq ID No. 6) showed the largest range and the CsgG:CsgF complex which contains the longest CsgF peptide (1-45 of Seq ID No. 6) showed the smallest range (FIG. 21).

Materials and Methods for Characterisation of Analytes:

The proteins produced by the methods described below can be used interchangeably with those produced by the methods described above with respect to structural determination.

### Methods

### Expression of the CsgG:CsgF or CsgG:FCP Complex by Co-Expression

Genes encoding the CsgG proteins and its mutants are constructed in the pT7 vector which contains ampicillin resistance gene. Genes encoding the CsgF or FCP proteins and its mutants are constructed in the pRham vector which contains Kanamycin resistant gene. lᵢlt of both plasmids is mixed with 50 μL of Lemo(DE3)ΔCsgEFG for 10 minutes on ice. The sample is then heated at 42° C. for 45 seconds before being returned to ice for another 5 minutes. 150 μL of NEB SOC outgrowth medium is added and the sample is incubated at 37° C. with shaking at 250 rpm for 1 hour. The entire volume is spread onto an agar plate containing kanamycin (40 ug/mL), ampicillin (100 ug/mL) and chloramphenicol (34 ug/ml) and incubated overnight at 37° C. Single colony is taken from the plate and inoculated into 100 mL of LB media containing kanamycin (40 ug/mL), ampicillin (100 ug/mL) and chloramphenicol (34 ug/ml) and incubated overnight at 37° C. with shaking at 250 rpm. 25 mL of the starter culture is added to 500 mL of LB media containing 3 mM ATP, 15 mM MgSO₄, kanamycin (40 ug/mL), ampicillin (100 ug/mL) and chloramphenicol (34 ug/ml) and incubated overnight at 37° C. The culture was allowed to grow for 7 hours, at which point the OD₆₀₀ was greater than 3.0. Lactose (1.0% final concentration), glucose (0.2% final concentration) and rhamnose (2 mM final concentration) were added and the temperature dropped to 18° C. whist shaking is maintained at 250 rpm for 16 hours. Culture was centrifuged at 6000 rpm for 20 mins at 4° C. The supernatant was discarded and the pellet kept. Cells stored at −80° C. until purification.

Expression of the CsgG Pore with or without a C-Term Strep Tag and CsgF with or without a C Terminal Strep or His Tag

All genes encoding all the CsgG proteins and CsgF or FCP proteins are constructed in the pT7 vector which contains ampicillin resistance gene. Expression procedure is same as above except for Kanacmycin is being omitted in all medias and buffers.

### Cell Lysis (Co Expressed Complex or Individual CsgG/CsgF/FCP Proteins)

The lysis buffer is made of 50 mM Tris, pH 8.0, 150 mM NaCl, 0.1% DDM, 1× Bugbuster Protein Extraction Reagent (Merck), 2.5 μL Benzonase Nuclease (stock ≥250 units/μL)/100 mL of lysis buffer and 1 tablet Sigma Protease inhibitor cocktail/100 mL of lysis buffer. 5× volume of lysis buffer is used to lyse 1× weight of harvested cells. Cells resuspended and left to spin at room temperature for 4 hours until a homogenous lysate is produced. Lysate is spun at 20,000 rpm for 35 minutes at 4° C. The supernatant is carefully extracted and filtered through a 0.2 uM Acrodisc syringe filter.

### Strep Purification of the CsgG or CsgF/FCP Proteins or Co-Expressed Complex if the CsgG Contains a C-Term Strep Tag and CsgF or FCP Contains a C-Term His Tag

The filtered sample was then loaded onto a 5mL StrepTrap column with the following parameters: Loading speed: 0.8 mL/min, Complete sample loading: 10 mL, Wash out unbound: 10CV (5 mL/min), Extra wash: 10CV (5 mL/min), Elution: 3CV (5 mL/min). Affinity buffer: 50 mL Tris, pH 8.0, 150 mM NaCl, 0.1% DDM; Wash buffer: 50 mL Tris, pH 8.0, 2M NaCl, 0.1% DDM; Elution buffer: 50 mL Tris, pH8.0, 150 mM NaCl, 0.1% DDM, 10 mM desthiobiotin.

Eluted sample is collected.

### His Purification of the CsgG or CsgF/FCP Proteins or Co-Expressed Complex if the CsgG Contains a C-Term Strep Tag and CsgF or FCP Contains a C-Term His Tag

Filtered sample or pooled eluted peaks from Strep purification (in case of the complex) loaded onto 5 mL HisTrap column using the same parameters as above, except with the following buffers: Affinity & wash buffer: 50 mL Tris, pH 8.0, 150 mM NaCl, 0.1% DDM, 25 mM imidazole; Elution: 50 mL Tris, pH 8.0, 150 mM NaCl, 0.1% DDM, 350 mM imidazole. Peak eluted, concentrated in 30 kDa MWCO Merck Milipore centrifugal unit to a volume of 500 uL.

Formation of the Complex In Vitro with In Vivo Purified Components.

Both the CsgG and the CsgF/FCP proteins expressed and purified separately are mixed in various ratios to identify the correct ratio. however always in excess CsgF conditions. The complex was then incubated overnight at 25° C. To remove the excess CsgF and remove DTT from the buffer, the mixture was again injected onto the Superdex Increase 200 10/300 equilibrated in 50 mM Tris, pH 8.0, 150 mM NaCl, 0.1% DDM. The complex usually elutes between 9 to 10 mL on this column.

Polishing Step with Gel Filtration for the Complex (Co-Expressed or Made In Vitro)

If necessary, Strep purified or His purified or His followed by Strep purified CsgG:CsgF or CsgG:FCP can be subjected to a further polishing step by gel filtration. 500 μL of the sample was injected into a 1 mL sample loop and onto the Superdex Increase 200 10/300 equilibrated in 50 mM Tris, pH 8.0, 150 mM NaCl, 0.1% DDM. The peak associated to the complex usually elutes between 9 and 10 mL on this column when run 1 mL/min. Sample was heated at 60° C. for 15 minutes and centrifuged at 21,000 rcf for 10 mins. Supernatant was taken for testing. Samples were subjected to SDS-PAGE to confirm and identify fractions eluted with the complex.

### Cleavage of CsgF or FCP at the TEV Protease Site

If the CsgF or FCP contains a TEV cleavage site, TEV-protease with a C-term Histidine tag is added to the sample (amount added is identified based on the rough concentration of the protein complex) with 2 mM DTT. Sample incubated overnight at 4° C. on the roller mixer at 25 rpm. The mixture is then run back through a 5 mL HisTrap column and the flow through is collected. Anything uncleaved will remain bound to the column and the cleaved protein will elute. Same buffers and parameters and the final heating step are used as in the His purification described above.

Purifying the CsgG:FCP Complex with In Vivo Purified CsgG Pore and Synthetic FCP

Lyophilised FCP peptides received from Genscript and Lifetein. 1mg of peptide dissolved in 1mL of nuclease free ddH₂O to obtain lmg/mL sample. Sample was vortexed until no peptide remains visible. Due to differences in expression levels of CsgG pores and mutants, it's difficult to measure the concentration accurately. Intensity of protein bands on SDS-PAGE against known markers can be used to get a rough estimate of the sample. CsgG and FCP are then mixed in approximately 1:50 molar ratio and incubate at 25° C. overnight at 700 rpm. Samples were heated at 60° C. for 15 minutes and centrifuged at 21,000rcf for 10 mins.

Supernatant was taken for testing. If needed, the complex can be purified as detailed above in co-expression.

### Purifying CsgG:CsgF or CsgG:FCP Containing Cysteine Mutants

Same procedure as above can be used to purify the CsgG:CsgF or CsgG:FCP complexes (with I or II or III below) if either or both components contain cysteines except for the composition of affinity, wash and elution buffers in His and Strep purifications and the buffer used in gel filtration. To purify cysteine mutants, all these buffers should contain 2 mM DTT. 2 mM DTT was also been added when synthetic peptides containing cysteines are dissolved in ddH₂O

I.co-expression of CsgG and CsgF or FCP

II. Making the CsgG:CsgF or CsgG:FCP complexes in vitro with in vivo purified individual components

III. Making the CsgG:CsgF or CsgG:FCP complexes in vitro with in vivo purified CsgG and synthetic FCP

### Determination of Cys-Bond Formation

Two tubes of 50 μL each from the final elution were separated. In one of the tube, 2 mM DTT was added as a reducing agent and in the other tube 100 μM of Cu(II):1-10 Phenanthroline (33 mM: 100 mM) was added as an oxidizing agent. Samples were mixed 1:1 with Laemmli buffer containing 4% SDS. Half the sample were heat treated to 100 deg for 10 min (denaturating condition) and half of them were left untreated, before running on a 4-20% TGX gel (Bio-rad Criterion) in TGS buffer.

### Coupled In Vitro Transcription and Translation (IVTT)

All proteins were generated by coupled in vitro transcription and translation (IVTT) by using an E. coli T7-S30 extract system for circular DNA (Promega). The complete 1 mM amino acid mixture minus cysteine and the complete 1 mM amino acid mixture. minus methionine were mixed in equal volumes to obtain the working amino acid solution required to generate high concentrations of the proteins. The amino acids (10 uL) were mixed with premix solution (40 uL), [35S]L-methionine (2 uL, 1175 Ci/mmol, 10 mCi/mL), plasmid DNA (16 uL, 400 ng/uL) and T7 S30 extract (30 uL) and rifampicin (2 uL, 20 mg/mL) to generate a 100 μL reaction of wiT proteins. Synthesis was carried out for 4 hours at 30° C. followed by overnight incubation at room temperature. If the CsgG:CsgF or CsgG FCP complexes were made in co-expression, plasmid DNAs encoding each component were mixed in equal amounts, and a portion of the mixture (16 uL) was used for IVTT. After incubation, the tube was centrifuged for 10 minutes at 22000 g, of which the supernatant was discarded. The resulting pellet was resuspended and washed in MBSA (10 mM MOPS, 1 mg/ml BSA pH7.4) and centrifuged again under the same conditions. The protein present in the pellet was re-suspended in 1× Laemmli sample buffer and run in 4-20% TGX gel at 300V for 25 min. The gel was then dried and exposed to Carestream® Kodak® BioMax® MR film overnight. The film was then processed and the protein in the gel visualized.

### Samples for Testing in MinIONs

All samples prior to testing are incubated with Brij58 (final concentration of 0.1%) for 10 minutes at room temperature before making up subsequent pore dilutions necessary for pore insertion.

### Method for Preparing and Running Static Strands

A set of polyA DNA strands (SS20 to SS38 of FIG. 24) in which one base is missing from the DNA backbone (iSpc3) is obtained by Integrated DNA Technologies (IDT). 3′ end of each of these strand also comprise a biotin modification. The static strands are incubated with monovalent streptavidin at room temperature for 20 minutes, resulting in the biotin bmdmg to the streptavidin. The streptavidin-static strand complex was diluted to 500 nM (B, FIG. 24) and 2 uM (C, FIG. 24) in 25 mM HEPES, 430 mM KCl, 30 mM ATP, 30 mM MgCl2, 2.15 mM EDTA, pH8 (known as RBFM). The residual current generated by each static strand is recorded in a MinION set up. MinIOn flow cells were flushed as per standard running protocols, and then the sequencing protocol was started with 1 minute static flicks. Initially 10 minutes of open pore recording was generated before 150 μL of the first streptavidin-static strand complex was added. After 10 minutes, 800 μL of RBFM was flushed through the flow cell before the next streptavidin-static strand complex was added. This process was repeated for all streptavidin-static strands. Once the final streptavidin-static strand complex had been incubated on the flow cell, 800 μL of RBFM was flushed through the flow cell and 10 minutes of open pore recording was generated before finishing the experiment.

### Method for Making Discrimination Profile Plots

The reader head discrimination profiles show the average variation in modelled current when the base at each reader head position is varied. To calculate the reader head discrimination at position i for a model of length k with alphabet of length n, we defined the discrimination at reader head position i as the median of the standard deviations in current level for each of the n^{k-1} groups of size n where position i is varied while other positions are held constant.

### Example 9: Pore Complex Models

Molecular modelling is powerful and accurate means of predicting the interactions of analytes with nanopores, and is extensively used in the field of nanopore sensing. It is particularly useful for predicting the geometry and distances between protein components and/or analytes. Molecular modelling has been used to accurately predict the positions of maximum discrimination for a polynucleotide in a nanopore complex. It is known in the art that the bases in a polynucleotide that are nearest to the narrowest points of the constriction regions of a nanopore are those which maximally alter the current flowing through the channel, and thus maximum discrimination is achieved at the constriction regions. By combining profile modelling (using HOLE) with modelling of polynucleotides that are extended through the channel we are able to accurately predict which bases in polynucleotide will maximally change the current flowing through the pore.

FIGS. 33-45 show molecular modelling results generated from pore complexes formed between different example transmembrane protein nanopores and auxiliary proteins. The transmembrane protein nanopores MspA, α-hemolysin (αHL) and CsgG were individually modelled with each of the ring-shaped auxiliary proteins CsgF peptide (FIG. 33), GroES (FIGS. 34, 37, 40, 43), pentraxin (FIGS. 36, 39, 42, 45), and SP1 (FIGS. 35, 38, 41, 44). CsgG was further modelled as a three-component pore complex with CsgF and a ring-shaped auxiliary protein (FIGS. 43-45).

Part A) of FIGS. 33-45 show modelling of single-stranded DNA extended through the channel of the pore complexes. Part B) shows the internal geometry profile of the channel, generated using HOLE mapping software. Part C) shows the profile generated from the HOLE software for the internal radius of the channel along the z-axis of the pore complex. Dotted lines marking the major constrictions in both the nanopore and the auxillary proteins are added to aid the eye. The modelling demonstrates for each pore complex that the transmembrane protein nanopore and auxiliary protein align to form a continuous channel comprising at least two constriction regions, in accordance with the present disclosure.

The modelling is able to predict the extent of discrimination from the radius of the constrictions, and also the nucleotide distance between the constriction points. Although the exact register of the polynucleotide in the channel of the pore complex is difficult to determine because it depends on the seating of the enzyme motor on top of the pore complex and the applied voltage (which affects the stretch of the polynucleotide), modelling gives a very good prediction of relative nucleotide distance between the peaks in discrimination. The modelling of the CsgG+CsgF-peptide complex predicted a distance of about 5-6 nucleotide between the maximums of discrimination from the CsgG and CsgF-peptide readers (FIG. 33), which was borne out by experimental electrical measurements of DNA discrimination in the fully assembled complex (FIGS. 24-25).

### Methodology:

The structures for MspA, aHL, CsgG, GroES, pentraxin and SP1 were taken from the Protein Data Bank (Protein Data Bank references as described above with reference to the description of the Figures). The CsgG/CsgF structure was obtained independently. Each auxiliary protein was modelled by being placed on top of each pore such that the distance between the proteins was minimised.

Pore radius profiles were generated using the publicly available software, HOLE (holeprogram.org/), to map the pore radius through each of the pore/auxiliary protein combinations.

Visualisations of the continuous channel through the pore/auxiliary protein combinations were generated using the output from the HOLE software along with the molecular visualisation package VMD (ks.uiuc.edu/Research/vmd/) to display the channel through each pore/auxiliary protein.

### Sequences

### Description of the Sequences:

SEQ ID NO:1 shows polynucleotide sequence of wild-type E. coli CsgG from strain K12, including signal sequence (Gene ID: 945619).

SEQ ID NO:2 shows amino acid sequence of wild-type E. coli CsgG including signal sequence (Uniprot accession number P0AEA2).

SEQ ID NO:3 shows amino acid sequence of wild-type E. coli CsgG as mature protein (Uniprot accession number P0AEA2).

SEQ ID NO:4 shows polynucleotide sequence of wild-type E. coli CsgF from strain K12, including signal sequence (Gene ID: 945622).

SEQ ID NO:5 shows amino acid sequence of wild-type E. coli CsgF including signal sequence (Uniprot accession number P0AE98).

SEQ ID NO:6 shows amino acid sequence of wild-type E. coli CsgF as mature protein (Uniprot accession number P0AE98).

SEQ ID NO:7 shows polynucleotide sequence of a fragment of wild-type E. coli CsgF encoding amino acids 1 to 27 and a C-terminal 6 His tag.

SEQ ID NO:8 shows amino acid sequence of a fragment of wild-type E. coli CsgF encompassing amino acids 1 to 27 and a C-terminal 6 His tag.

SEQ ID NO:9 shows polynucleotide sequence of a fragment of wild-type E. coli CsgF encoding amino acids 1 to 38 and a C-terminal 6 His tag.

SEQ ID NO:10 shows amino acid sequence of a fragment of wild-type E. coli CsgF encompassing amino acids 1 to 38 and a C-terminal 6 His tag.

SEQ ID NO:11 shows polynucleotide sequence of a fragment of wild-type E. coli CsgF encoding amino acids 1 to 48 and a C-terminal 6 His tag.

SEQ ID NO:12 shows amino acid sequence of a fragment of wild-type E. coli CsgF encompassing amino acids 1 to 48 and a C-terminal 6 His tag.

SEQ ID NO:13 shows polynucleotide sequence of a fragment of wild-type E. coli CsgF encoding amino acids 1 to 64 and a C-terminal 6 His tag.

SEQ ID NO:14 shows amino acid sequence of a fragment of wild-type E. coli CsgF encompassing amino acids 1 to 64 and a C-terminal 6 His tag.

SEQ ID NO:15 shows amino acid sequence of a peptide corresponding to residues 20 to 53 of E. coli CsgF

SEQ ID NO:16 shows amino acid sequence of a peptide corresponding to residues 20 to 42 of E. coli CsgF, including KD at its C-terminus

SEQ ID NO:17 shows amino acid sequence of a peptide corresponding to residues 23 to 55 of CsgF homologue Q88H88

SEQ ID NO:18 shows amino acid sequence of a peptide corresponding to residues 25 to 57 of CsgF homologue A0A143HJA0

SEQ ID NO:19 shows amino acid sequence of a peptide corresponding to residues 21 to 53 of CsgF homologue Q5E245

SEQ ID NO:20 shows amino acid sequence of a peptide corresponding to residues 19 to 51 of CsgF homologue Q084E5

SEQ ID NO:21 shows amino acid sequence of a peptide corresponding to residues 15 to 47 of CsgF homologue F0LZU2

SEQ ID NO:22 shows amino acid sequence of a peptide corresponding to residues 26 to 58 of CsgF homologue A0A136HQR0

SEQ ID NO:23 shows amino acid sequence of a peptide corresponding to residues 21 to 53 of CsgF homologue A0A0W1SRL3

SEQ ID NO:24 shows amino acid sequence of a peptide corresponding to residues 26 to 59 of CsgF homologue B0UH01

SEQ ID NO:25 shows amino acid sequence of a peptide corresponding to residues 22 to 53 of CsgF homologue Q6NAU5

SEQ ID NO:26 shows amino acid sequence of a peptide corresponding to residues 7 to 38 of CsgF homologue G8PUY5

SEQ ID NO:27 shows amino acid sequence of a peptide corresponding to residues 25 to 57 of CsgF homologue A0A0S2ETP7

SEQ ID NO:28 shows amino acid sequence of a peptide corresponding to residues 19 to 51 of CsgF homologue E3I1Z1

SEQ ID NO:29 shows amino acid sequence of a peptide corresponding to residues 24 to 55 of CsgF homologue F3Z094

SEQ ID NO:30 shows amino acid sequence of a peptide corresponding to residues 21 to 53 of CsgF homologue A0A176T7M2

SEQ ID NO:31 shows amino acid sequence of a peptide corresponding to residues 14 to 45 of CsgF homologue D2QPP8

SEQ ID NO:32 shows amino acid sequence of a peptide corresponding to residues 28 to 58 of CsgF homologue N2IYT1

SEQ ID NO:33 shows amino acid sequence of a peptide corresponding to residues 26 to 58 of CsgF homologue W7QHV5

SEQ ID NO:34 shows amino acid sequence of a peptide corresponding to residues 23 to 55 of CsgF homologue D4ZLW2

SEQ ID NO:35 shows amino acid sequence of a peptide corresponding to residues 21 to 53 of CsgF homologue D2QT92

SEQ ID NO:36 shows amino acid sequence of a peptide corresponding to residues 20 to 51 of CsgF homologue A0A167UJA2

SEQ ID NO:37 shows amino acid sequence of a fragment of wild-type E. coli CsgF encompassing amino acids 20 to 27.

SEQ ID NO:38 shows amino acid sequence of a fragment of wild-type E. coli CsgF encompassing amino acids 20 to 38.

SEQ ID NO:39: shows amino acid sequence of a fragment of wild-type E. coli CsgF encompassing amino acids 20 to 48.

SEQ ID NO:40 shows amino acid sequence of a fragment of wild-type E. coli CsgF encompassing amino acids 20 to 64.

SEQ ID NO:41 shows the nucleotide sequence of primer CsgF_d27_end

SEQ ID NO:42 shows the nucleotide sequence of primer CsgF_d38_end

SEQ ID NO:43 shows the nucleotide sequence of primer CsgF_d48_end

SEQ ID NO:44 shows the nucleotide sequence of primer CsgF_d64_end

SEQ ID NO:45 shows the nucleotide sequence of primer pNa62_CsgF_histag_Fw

SEQ ID NO:46 shows the nucleotide sequence of primer CsgF-His_pET22b_FW

SEQ ID NO:47 shows the nucleotide sequence of primer CsgF-His_pET22b_Rev

SEQ ID NO:48 shows the nucleotide sequence of primer csgEFG_pDONR221_FW

SEQ ID NO:49 shows the nucleotide sequence of primer csgEFG_pDONR221_Rev

SEQ ID NO:50 shows the nucleotide sequence of primer Mut_csgF_His_FW

SEQ ID NO:51 shows the nucleotide sequence of primer Mut_csgF_His_Rev

SEQ ID NO:52 shows the nucleotide sequence of primer DelCsgE_Rev

SEQ ID NO:53 shows the nucleotide sequence of primer DelCsgE FW

SEQ ID NO: 54 shows the amino acid sequence of residues 1 to 30 of mature E. coli CsgF

SEQ ID NO: 55 shows the amino acid sequence of residues 1 to 35 of mature E. coli CsgF

SEQ ID NO: 56 shows the amino acid sequence of a mutated (T4C/N17S) CsgF sequence with a signal sequence, and a TEV protease cleavage site (ENLYFQS) inserted between residues 35 and 36 of sequence of the mature protein.

SEQ ID NO: 57 shows the amino acid sequence of a mutated (N17S-Del) CsgF sequence with a signal sequence, and a TEV protease cleavage site (ENLYFQS) inserted between residues 35 and 36 of sequence of the mature protein.

SEQ ID NO: 58 shows the amino acid sequence of a mutated (G1C/N17S) CsgF sequence with a signal sequence, and a TEV protease cleavage site (ENLYFQS) inserted between residues 35 and 36 of sequence of the mature protein.

SEQ ID NO: 59 shows the amino acid sequence of a mutated (G1C) CsgF sequence with a signal sequence, and a TEV protease cleavage site (ENLYFQS) inserted between residues 35 and 36 of sequence of the mature protein.

SEQ ID NO: 60 shows the amino acid sequence of a CsgF sequence with a signal sequence, a TEV protease cleavage site (ENLYFQS) inserted between residues 45 and 46 of sequence of the mature protein, and a His₁₀ tag at the C-terminus.

SEQ ID NO: 61 shows the amino acid sequence of a CsgF sequence with a signal sequence, a TEV protease cleavage site (ENLYFQS) inserted between residues 35 and 36 of sequence of the mature protein, and a His₁₀ tag at the C-terminus.

SEQ ID NO: 62 shows the amino acid sequence of a CsgF sequence with a signal sequence, a TEV protease cleavage site (ENLYFQS) inserted between residues 30 and 31 of sequence of the mature protein, and a His₁₀ tag at the C-terminus.

SEQ ID NO: 63 shows the amino acid sequence of a CsgF sequence with a signal sequence, a TEV protease cleavage site (ENLYFQS) inserted between residues 45 and 51 of sequence of the mature protein, and a His₁₀ tag at the C-terminus.

SEQ ID NO: 64 shows the amino acid sequence of a CsgF sequence with a signal sequence, a TEV protease cleavage site (ENLYFQS) inserted between residues 30 and 37 of sequence of the mature protein, and a His₁₀ tag at the C-terminus.

SEQ ID NO: 65 shows the amino acid sequence of a CsgF sequence with a signal sequence, a HCV C3 protease cleavage site (LEVLFQGP) inserted between residues 34 and 36 of sequence of the mature protein, and a His₁₀ tag at the C-terminus.

SEQ ID NO: 66 shows the amino acid sequence of a CsgF sequence with a signal sequence, a HCV C3 protease cleavage site (LEVLFQGP) inserted between residues 42 and 43 of sequence of the mature protein, and a His₁₀ tag at the C-terminus.

SEQ ID NO: 67 shows the amino acid sequence of a CsgF sequence with a signal sequence, a HCV C3 protease cleavage site (LEVLFQGP) inserted between residues 38 and 47 of sequence of the mature protein, and a His₁₀ tag at the C-terminus.

SEQ ID NO: 68 shows the amino acid sequence of YP_001453594.1: 1-248 of hypothetical protein CKO_02032 [Citrobacter koseri ATCC BAA-895], which is 99% identical to SEQ ID NO: 3.

SEQ ID NO: 69 shows the amino acid sequence of WP_001787128.1: 16-238 of curli production assembly/transport component CsgG, partial [Salmonella enterica], which is 98% to SEQ ID NO: 3.

SEQ ID NO: 70 shows the amino acid sequence of KEY44978.1|: 16-277 of curli production assembly/transport protein CsgG [Citrobacter amalonaticus], which is 98% identical to SEQ ID NO: 3.

SEQ ID NO: 71 shows the amino acid sequence of YP_003364699.1: 16-277 of curli production assembly/transport component [Citrobacter rodentium ICC168], which is 97% identical to SEQ ID NO: 3.

SEQ ID NO: 72 shows the amino acid sequence of YP_004828099.1: 16-277 of curli production assembly/transport component CsgG [Enterobacter asburiae LF7a], which is 94% identical to SEQ ID NO: 3.

SEQ ID NO: 73 shows the amino acid sequence of WP_006819418.1: 19-280 of transporter [Yokenella regensburgei], which is 91% identical to SEQ ID NO: 3.

SEQ ID NO: 74 shows the amino acid sequence of WP_024556654.1: 16-277 of curli production assembly/transport protein CsgG [Cronobacter pulveris], which is 89% identical to SEQ ID NO: 3.

SEQ ID NO: 75 shows the amino acid sequence of YP_005400916.1 :16-277 of curli production assembly/transport protein CsgG [Rahnella aquatilis HX2], which is 84% identical to SEQ ID NO: 3.

SEQ ID NO: 76 shows the amino acid sequence of KFC99297.1: 20-278 of CsgG family curli production assembly/transport component [Kluyvera ascorbata ATCC 33433], which is 82% identical to SEQ ID NO: 3.

SEQ ID NO: 77 shows the amino acid sequence of KFC86716.11:16-274 of CsgG family curli production assembly/transport component [Hafnia alvei ATCC 13337], which is 81% identical to SEQ ID NO: 3.

SEQ ID NO: 78 shows the amino acid sequence of YP_007340845.1|:16-270 of uncharacterised protein involved in formation of curli polymers [Enterobacteriaceae bacterium strain FGI 57], which is 76% identical to SEQ ID NO: 3.

SEQ ID NO: 79 shows the amino acid sequence of WP_010861740.1: 17-274 of curli production assembly/transport protein CsgG [Plesiomonas shigelloides], which is 70% identical to SEQ ID NO: 3.

SEQ ID NO: 80 shows the amino acid sequence of YP_205788.1 : 23-270 of curli production assembly/transport outer membrane lipoprotein component CsgG [Vibrio fischeri ES114], which is 60% identical to SEQ ID NO: 3.

SEQ ID NO: 81 shows the amino acid sequence of WP_017023479.1: 23-270 of curli production assembly protein CsgG [Aliivibrio logei], which is 59% identical to SEQ ID NO: 3.

SEQ ID NO: 82 shows the amino acid sequence of WP_007470398.1: 22-275 of Curli production assembly/transport component CsgG [Photobacterium sp. AK15], which is 57% identical to SEQ ID NO: 3.

SEQ ID NO: 83 shows the amino acid sequence of WP_021231638.1: 17-277 of curli production assembly protein CsgG [Aeromonas veronii], which is 56% identical to SEQ ID NO: 3.

SEQ ID NO: 84 shows the amino acid sequence of WP_033538267.1: 27-265 of curli production assembly/transport protein CsgG [Shewanella sp. ECSMB14101], which is 56% identical to SEQ ID NO: 3.

SEQ ID NO: 85 shows the amino acid sequence of WP_003247972.1: 30-262 of curli production assembly protein CsgG [Pseudomonas putida], which is 54% identical to SEQ ID NO: 3.

SEQ ID NO: 86 shows the amino acid sequence of YP_003557438.1: 1-234 of curli production assembly/transport component CsgG [Shewanella violacea DSS12], which is 53% identical to SEQ ID NO: 3.

SEQ ID NO: 87 shows the amino acid sequence of WP_027859066.1: 36-280 of curli production assembly/transport protein CsgG [Marinobacterium jannaschii], which is 53% identical to SEQ ID NO: 3.

SEQ ID NO: 88 shows the amino acid sequence of CEJ70222.1: 29-262 of Curli production assembly/transport component CsgG [Chryseobacterium oranimense G311], which is 50% identical to SEQ ID NO: 3.

SEQ ID NO: 89 shows the DNA sequence encoding Pro-CP1-Eco-(WT-Y51A/F56Q/D149N/E185N/E201N/E203N-StrepII(C))).

SEQ ID NO: 90 shows the DNA sequence encoding Pro-CP1-Eco-(WT-Y51A/F56Q/D149N/E185N/E201N/E203N-StrepII(C))).

SEQ ID NO: 1 (>P0AEA2; coding sequence for WT CsgG from E. coli K12)
ATGCAGCGCTTATTTCTTTTGGTTGCCGTCATGTTACTGAGCGGATGCTTAACCGCCCCGCCTAAAGAAGCCGCCA
GACCGACATTAATGCCTCGTGCTCAGAGCTACAAAGATTTGACCCATCTGCCAGCGCCGACGGGTAAAATCTTTGT
TTCGGTATACAACATTCAGGACGAAACCGGGCAATTTAAACCCTACCCGGCAAGTAACTTCTCCACTGCTGTTCCG
CAAAGCGCCACGGCAATGCTGGTCACGGCACTGAAAGATTCTCGCTGGTTTATACCGCTGGAGCGCCAGGGCTTA
CAAAACCTGCTTAACGAGCGCAAGATTATTCGTGCGGCACAAGAAAACGGCACGGTTGCCATTAATAACCGAATC
CCGCTGCAATCTTTAACGGCGGCAAATATCATGGTTGAAGGTTCGATTATCGGTTATGAAAGCAACGTCAAATCTG
GCGGGGTTGGGGCAAGATATTTTGGCATCGGTGCCGACACGCAATACCAGCTCGATCAGATTGCCGTGAACCTGC
GCGTCGTCAATGTGAGTACCGGCGAGATCCTTTCTTCGGTGAACACCAGTAAGACGATACTTTCCTATGAAGTTCA
GGCCGGGGTTTTCCGCTTTATTGACTACCAGCGCTTGCTTGAAGGGGAAGTGGGTTACACCTCGAACGAACCTGTT
ATGCTGTGCCTGATGTCGGCTATCGAAACAGGGGTCATTTTCCTGATTAATGATGGTATCGACCGTGGTCTGTGGG
ATTTGCAAAATAAAGCAGAACGGCAGAATGACATTCTGGTGAAATACCGCCATATGTCGGTTCCACCGGAATCCT
GA
SEQ ID NO: 2 (>P0AEA2 (1:277); WT prepro CsgG from E. coli K12)
MQRLFLLVAVMLLSGCLTAPPKEAARPTLMPRAQSYKDLTHLPAPTGKIFVSVYNIQDETGQFKPYPASNFSTAVPQ
SATAMLVTALKDSRWFIPLERQGLQNLLNERKIIRAAQENGTVAINNRIPLQSLTAANIMVEGSIIGYESNVKSGGV
GARYFGIGADTQYQLDQIAVNLRVVNVSTGEILSSVNTSKTILSYEVQAGVFRFIDYQRLLEGEVGYTSNEPVMLCL
MSAIETGVIFLINDGIDRGLWDLQNKAERQNDILVKYRHMSVPPES
SEQ ID NO: 3 (>P0AEA2 (16:277); mature CsgG from E. coli K12)
CLTAPPKEAARPTLMPRAQSYKDLTHLPAPTGKIFVSVYNIQDETGQFKPYPASNFSTAVPQSATAMLVTALKDSRW
FIPLERQGLQNLLNERKIIRAAQENGTVAINNRIPLQSLTAANIMVEGSIIGYESNVKSGGVGARYFGIGADTQYQL
DQIAVNLRVVNVSTGEILSSVNTSKTILSYEVQAGVFRFIDYQRLLEGEVGYTSNEPVMLCLMSAIETGVIFLINDG
IDRGLWDLQNKAERQNDILVKYRHMSVPPES
SEQ ID NO: 4 (>P0AE98; coding sequence for WT CsgF from E. coli K12)
ATGCGTGTCAAACATGCAGTAGTTCTACTCATGCTTATTTCGCCATTAAGTTGGGCTGGAACCATGACTTTCCAGTT
CCGTAATCCAAACTTTGGTGGTAACCCAAATAATGGCGCTTTTTTATTAAATAGCGCTCAGGCCCAAAACTCTTATA
AAGATCCGAGCTATAACGATGACTTTGGTATTGAAACACCCTCAGCGTTAGATAACTTTACTCAGGCCATCCAGTC
ACAAATTTTAGGTGGGCTACTGTCGAATATTAATACCGGTAAACCGGGCCGCATGGTGACCAACGATTATATTGTC
GATATTGCCAACCGCGATGGTCAATTGCAGTTGAACGTGACAGATCGTAAAACCGGACAAACCTCGACCATCCAG
GTTTCGGGTTTACAAAATAACTCAACCGATTTT
SEQ ID NO: 5 (>P0AE98 (1:138); WT pre CsgF from E. coli K12)
MRVKHAVVLLMLISPLSWAGTMTFQFRNPNFGGNPNNGAFLLNSAQAQNSYKDPSYNDDFGIETPSALDNFTQAIQS
QILGGLLSNINTGKPGRMVTNDYIVDIANRDGQLQLNVTDRKTGQTSTIQVSGLQNNSTDF
SEQ ID NO: 6 (>P0AE98 (20:138); WT mature CsgF from E. coli K12)
GTMTFQFRNPNFGGNPNNGAFLLNSAQAQNSYKDPSYNDDFGIETPSALDNFTQAIQSQILGGLLSNINTGKPGRMV
TNDYIVDIANRDGQLQLNVTDRKTGQTSTIQVSGLQNNSTDF
SEQ ID NO: 7 (>P0AE98; coding sequence for CsgF 1:27_6His)
ATGCGTGTCAAACATGCAGTAGTTCTACTCATGCTTATTTCGCCATTAAGTTGGGCTGGAACCATGACTTTCCAGTT
CCGTCATCACCATCACCATCACTAAGCCC
SEQ ID NO: 8 (>P0AE98 (1:28); preprotein of CsgF 20:27_6His)
MRVKHAVVLLMLISPLSWA GTMTFQFR HHHHHH
SEQ ID NO: 9 (>P0AE98; coding sequence for CsgF 1:38_6His)
ATGCGTGTCAAACATGCAGTAGTTCTACTCATGCTTATTTCGCCATTAAGTTGGGCTGGAACCATGACTTTCCAGTT
CCGTAATCCAAACTTTGGTGGTAACCCAAATAATGGCCATCACCATCACCATCACTAAGCCC
SEQ ID NO: 10 (>P0AE98 (1:39); preprotein of CsgF 20:38_6His)
MRVKHAVVLLMLISPLSWAGTMTFQFRNPNFGGNPNNG HHHHHH
SEQ ID NO: 11 (>P0AE98; coding sequence for CsgF 1:48_6His)
ATGCGTGTCAAACATGCAGTAGTTCTACTCATGCTTATTTCGCCATTAAGTTGGGCTGGAACCATGACTTTCCAGTT
CCGTAATCCAAACTTTGGTGGTAACCCAAATAATGGCGCTTTTTTATTAAATAGCGCTCAGGCCCAACATCACCATC
ACCATCACTAAGCCC
SEQ ID NO: 12 (>P0AE98 (1:49); preprotein of CsgF 20:48_6His)
MRVKHAVVLLMLISPLSWAGTMTFQFRNPNFGGNPNNGAFLLNSAQAQ HHHHHH
SEQ ID NO: 13 (>P0AE98; coding sequence for CsgF 1:64_6His)
ATGCGTGTCAAACATGCAGTAGTTCTACTCATGCTTATTTCGCCATTAAGTTGGGCTGGAACCATGACTTTCCAGTT
CCGTAATCCAAACTTTGGTGGTAACCCAAATAATGGCGCTTTTTTATTAAATAGCGCTCAGGCCCAAAACTCTTATA
AAGATCCGAGCTATAACGATGACTTTGGTATTGAAACA CATCACCATCACCATCACTAAGCCC
SEQ ID NO: 14 (>P0AE98 (1:65); preprotein of CsgF 20:64_6His)
MRVKHAVVLLMLISPLSWAGTMTFQFRNPNFGGNPNNGAFLLNSAQAQNSYKDPSYNDDFGIETHHHHHH
SEQ ID NO: 15 (>P0AE98 (20:53); mature peptide of CsgF 20:53)
GTMTFQFRNPNFGGNPNNGAFLLNSAQAQNSYKD
SEQ ID NO: 16 (>P0AE98 (20:42); mature peptide of CsgF 20:42 + KD)
GTMTFQFRNPNFGGNPNNGAFLLKD
SEQ ID NO: 17 (>Q88H88_PSEPK (23:55))
TELVYTPVNPAFGGNPLNGTWLLNNAQAQNDY
SEQ ID NO: 18 (>A0A143HJA0_9GAMM (25:57))
TELIYEPVNPNFGGNPLNGSYLLNNAQAQDRH
SEQ ID NO: 19 (>Q5E245_VIBF1 (21:53))
SELVYTPVNPNFGGNPLNTSHLFGGANAINDY
SEQ ID NO: 20 (>Q084E5_SHEFN (19:51))
TQLVYTPVNPAFGGSYLNGSYLLANASAQNEH
SEQ ID NO: 21 (>F0LZU2_VIBFN (15:47))
SSLVYEPVNPTFGGNPLNTTHLFSRAEAINDY
SEQ ID NO: 22 (>A0A136HQR0_9ALTE (26:58))
TELVYEPINPSFGGNPLNGSFLLSKANSQNAH
SEQ ID NO: 23 (>A0A0W1SRL3_9GAMM (21:53))
TEIVYQPINPSFGGNPMNGSFLLQKAQSQNAH
SEQ ID NO: 24 (>B0UH01_METS4 (26:59))
SSLVYQPVNPAFGGPQLNGSWLQAEANAQNIPQ
SEQ ID NO: 25 (>Q6NAU5_RHOPA (22:53))
GSLVYTPTNPAFGGSPLNGSWQMQQATAGNH
SEQ ID NO: 26 (>G8PUY5_PSEUV (7:38))
QQLIYQPTNPSFGGYAANTTHLFATANAQKTA
SEQ ID NO: 27 (>A0A0S2ETP7_9RHIZ (25:57))
GDLVYTPVNPSFGGSPLNSAHLLSIAGAQKNA
SEQ ID NO: 28 (>E3I1Z1_RHOVT (19:51))
AELGYTPVNPSFGGSPLNGSTLLSEASAQKPN
SEQ ID NO: 29 (>F3Z094_DESAF (24:55))
TELVFSFTNPSFGGDPMIGNFLLNKADSQKR
SEQ ID NO: 30 (>A0A176T7M2_9FLAO (21:53))
QQLVYKSINPFFGGGDSFAYQQLLASANAQND
SEQ ID NO: 31 (>D2QPP8_SPILD (14:45))
QALVYHPNNPAFGGNTFNYQWMLSSAQAQDR
SEQ ID NO: 32 (>N2IYT1_9PSED (26:58))
TELVYTPKNPAFGGSPLNGSYLLGNAQAQNDY
SEQ ID NO: 33 (>W7QHV5_9GAMM (26:58))
GQLIYQPINPSFGGDPLLGNHLLNKAQAQDTK
SEQ ID NO: 34 (>D4ZLW2_SHEVD (23:55))
TQLIYTPVNPNFGGSYLNGSYLLANASVQNDH
SEQ ID NO: 35 (>D2QT92_SPILD (21:53))
QAFVYHPNNPNFGGNTFNYSWMLSSAQAQDRT
SEQ ID NO: 36 (>A0A167UJA2_9FLAO (20:51))
QGLIYKPKNPAFGGDTFNYQWLASSAESQNK
SEQ ID NO: 37 (>P0AE98 (20:28); mature peptide of CsgF 20:27)
GTMTFQFR
SEQ ID NO: 38 (>P0AE98 (20:39); mature peptide of CsgF 20:38)
GTMTFQFRNPNFGGNPNNG
SEQ ID NO: 39 (>P0AE98 (20:49); mature peptide of CsgF 20:48)
GTMTFQFRNPNFGGNPNNGAFLLNSAQAQ
SEQ ID NO: 40 (>P0AE98 (20:65); mature peptide of CsgF 20:64)
GTMTFQFRNPNFGGNPNNGAFLLNSAQAQNSYKDPSYNDDFGIET
SEQ ID NO: 41 (CsgF_d27_end)
ACGGAACTGGAAAGTCATGGTTCC
SEQ ID NO: 42 (CsgF_d38_end)
GCCATTATTTGGGTTACCACCAAAGTTTGG
SEQ ID NO: 43 (CsgF_d48_end)
TTGGGCCTGAGCGCTATTTAATAAAAAAGC
SEQ ID NO: 44 (CsgF_d64_end)
TGTTTCAATACCAAAGTCATCGTTATAGCTCGG
SEQ ID NO: 45 (pNa62_CsgF_histag_Fw)
CATCACCATCACCATCACTAAGCCC
SEQ ID NO: 46 (CsgF-His_pET22b_FW)
CCCCCATATGGGAACCATGACTTTCCAGTTCC
SEQ ID NO: 47: (CsgF-His_pET22b_Rev)
CCCCGAATTCCTAATGGTGATGGTGATGGTGGTAAAAATCGGTTGAGTTATTTTG
SEQ ID NO: 48: (csgEFG_pDONR221_FW)
GGGGACAAGTTTGTACAAAAAAGCAGGCTACCTCAGGCGATAAAGCCATGAAACGTTA
SEQ ID NO: 49: (csgEFG_pDONR221_Rev)
GGGGACCACTTTGTACAAGAAAGCTGGGTGTTTAAACTCATTTTTCGAACTGCGGGTGGCTCCAAGCGCTGG
SEQ ID NO: 50: (Mut_csgF_His_FW)
CAAAATAACTCAACCGATTTTCATCACCATCACCATCACTAAGCCCCAGCTTCATAAGG
SEQ ID NO: 51: (Mut_csgF_His_Rev)
CCTTATGAAGCTGGGGCTTAGTGATGGTGATGGTGATGAAAATCGGTTGAGTTATTTTG
SEQ ID NO: 52: (DelCsgE_Rev)
AGCCTGCTTTTTTGTACAAAC
SEQ ID NO: 53: (DelCsgE FW)
ATAAAAAATTGTTCGGAGGCTGC
SEQ ID NO: 54 (>P0AE98 (20:50); mature peptide of CsgF 1:30)
GTMTFQFRNPNFGGNPNNGAFLLNSAQAQN
SEQ ID NO: 55 (>P0AE98 (20:54); mature peptide of CsgF 1:35)
GTMTFQFRNPNFGGNPNNGAFLLNSAQAQNSYKDP

Examples of CsgF sequences with protease cleavage sites made into proteins. Signal peptide is shown in bold TEV protease cleavage site in bold and underline and HCV C3 protease cleavage site in underline. StrepII indicate the Strep tag at the C terminus, H10 indicates the 10× Histidine tag at the C terminus and ** indicates STOP codons.

Pro-CsgF-Eco-(WT-T4C/N17S/P35-TEV-S36)-StrepII
SEQ ID NO: 56
MRVKHAVVLLMLISPLSWAGTMCFQFRNPNFGGNPSNGAFLLNSAQAQNSYKDPENLYFQSSYNDDFGIETPSALD
NFTQAIQSQILGGLLSNINTGKPGRMVTNDYIVDIANRDGQLQLNVTDRKTGQTSTIQVSGLQNNSTDFSAWSHPQF
EK**
Pro-CsgF-Eco-(WT-N17S-Del(P35-[TEV]-S36)-StrepII
SEQ ID NO: 57
MRVKHAVVLLMLISPLSWAGTMTFQFRNPNFGGNPSNGAFLLNSAQAQNSYKDPENLYFQSSYNDDFGIETPSALD
NFTQAIQSQILGGLLSNINTGKPGRMVTNDYIVDIANRDGQLQLNVTDRKTGQTSTIQVSGLQNNSTDFSAWSHPQF
EK**
Pro-CsgF-Eco-(WT-G1C/N17S/P35-[TEV]-S36)-StrepII
SEQ ID NO: 58
MRVKHAVVLLMLISPLSWACTMTFQFRNPNFGGNPSNGAFLLNSAQAQNSYKDPENLYFQSSYNDDFGIETPSALD
NFTQAIQSQILGGLLSNINTGKPGRMVTNDYIVDIANRDGQLQLNVTDRKTGQTSTIQVSGLQNNSTDFSAWSHPQF
EK**
Pro-CsgF-Eco-(WT-G1C/P35-[TEV]-S36)-StrepII
SEQ ID NO: 59
MRVKHAVVLLMLISPLSWACTMTFQFRNPNFGGNPNNGAFLLNSAQAQNSYKDPENLYFQSSYNDDFGIETPSALD
NFTQAIQSQILGGLLSNINTGKPGRMVTNDYIVDIANRDGQLQLNVTDRKTGQTSTIQVSGLQNNSTDFSAWSHPQF
EK**
Pro-CsgF-Eco-(WT-T45-TEV-P46)-H10
SEQ ID NO: 60
MRVKHAVVLLMLISPLSWAGTMTFQFRNPNFGGNPNNGAFLLNSAQAQNSYKDPSYNDDFGIETENLYFQSPSALD
NFTQAIQSQILGGLLSNINTGKPGRMVTNDYIVDIANRDGQLQLNVTDRKTGQTSTIQVSGLQNNSTDFHHHHHHHH
HH**
Pro-CsgF-Eco-(WT-P35-TEV-S36)-H10
SEQ ID NO: 61
MRVKHAVVLLMLISPLSWAGTMTFQFRNPNFGGNPNNGAFLLNSAQAQNSYKDPENLYFQSSYNDDFGIETPSALD
NFTQAIQSQILGGLLSNINTGKPGRMVTNDYIVDIANRDGQLQLNVTDRKTGQTSTIQVSGLQNNSTDFHHHHHHHH
HH**
Pro-CsgF-Eco-(WT-N30-TEV-S31)-H10
SEQ ID NO: 62
MRVKHAVVLLMLISPLSWAGTMTFQFRNPNFGGNPNNGAFLLNSAQAQNENLYFQSSYKDPSYNDDFGIETPSALD
NFTQAIQSQILGGLLSNINTGKPGRMVTNDYIVDIANRDGQLQLNVTDRKTGQTSTIQVSGLQNNSTDFHHHHHHHH
HH**
Pro-CsgF-Eco-(WT-T45-TEV-F51)-H10
SEQ ID NO: 63
MRVKHAVVLLMLISPLSWAGTMTFQFRNPNFGGNPNNGAFLLNSAQAQNSYKDPSYNDDFGIETENLYFQSFTQAI
QSQILGGLLSNINTGKPGRMVTNDYIVDIANRDGQLQLNVTDRKTGQTSTIQVSGLQNNSTDFHHHHHHHHHH**
Pro-CsgF-Eco-(WT-N30-TEV-Y37)-H10
SEQ ID NO: 64
MRVKHAVVLLMLISPLSWAGTMTFQFRNPNFGGNPNNGAFLLNSAQAQNENLYFQSYNDDEGIETPSALDNFTQAI
QSQILGGLLSNINTGKPGRMVTNDYIVDIANRDGQLQLNVTDRKTGQTSTIQVSGLQNNSTDFHHHHHHHHHH**
Pro-CsgF-Eco-(WT-D34-[C3]-S36)
SEQ ID NO: 65
MRVKHAVVLLMLISPLSWACTMTFQFRNPNFGGNPNNGAFLLNSAQAQNSYKDLEVLFQGPSYNDDFGIETPSALD
NFTQAIQSQILGGLLSNINTGKPGRMVTNDYIVDIANRDGQLQLNVTDRKTGQTSTIQVSGLQNNSTDFSAWSHPQF
EK**
Pro-CsgF-Eco-(WT-I42-[C3]-E43)
SEQ ID NO: 66
MRVKHAVVLLMLISPLSWACTMTFQFRNPNFGGNPNNGAFLLNSAQAQNSYKDPSYNDDFGILEVLFQGPETPSAL
DNFTQAIQSQILGGLLSNINTGKPGRMVTNDYIVDIANRDGQLQLNVTDRKTGQTSTIQVSGLQNNSTDFSAWSHPQ
FEK**
Pro-CsgF-Eco-(WT-N38-[C3]-S47)
SEQ ID NO: 67
MRVKHAVVLLMLISPLSWACTMTFQFRNPNFGGNPNNGAFLLNSAQAQNSYKDPSYNLEVLFQGPSALDNFTQAIQ
SQILGGLLSNINTGKPGRMVTNDYIVDIANRDGQLQLNVTDRKTGQTSTIQVSGLQNNSTDFSAWSHPQFEK**
SEQ ID NO: 68
MPRAQSYKDLTHLPMPTGKIFVSVYNIQDETGQFKPYPASNFSTAVPQSATAMLVTALKDSRWFIPLERQGLQNLLN
ERKIIRAAQENGTVAINNRIPLQSLTAANIMVEGSIIGYESNVKSGGVGARYFGIGADTQYQLDQIAVNLRVVNVST
GEILSSVNTSKTILSYEVQAGVFRFIDYQRLLEGEIGYTSNEPVMLCLMSAIETGVIFLINDGIDRGLWDLQNKAER
QNDILVKYRHMSVPPES
SEQ ID NO: 69
CLTAPPKQAAKPTLMPRAQSYKDLTHLPAPTGKIFVSVYNIQDETGQFKPYPASNFSTAVPQSATAMLVTALKDSRW
FIPLERQGLQNLLNERKIIRAAQENGTVAMNNRIPLQSLTAANIMVEGSIIGYESNVKSGGVGARYFGIGADTQYQL
DQIAVNLRVVNVSTGEILSSVNTSKTILSYEVQAGVFRFIDYQRLLEGEIGYTSNEPVMLCLMSAIETG
SEQ ID NO: 70
CLTAPPKEAAKPTLMPRAQSYKDLTHLPIPTGKIFVSVYNIQDETGQFKPYPASNFSTAVPQSATAMLVTALKDSRW
FVPLERQGLQNLLNERKIIRAAQENGTVAINNRIPLQSLTAANIMVEGSIIGYESNVKSGGVGARYFGIGADTQYQL
DQIAVNLRVVNVSTGEILSSVNTSKTILSYEVQAGVFRFIDYQRLLEGEIGYTSNEPVMLCLMSAIETGVIFLINDG
IDRGLWDLQNKADRQNDILVKYRHMSVPPES
SEQ ID NO: 71
CLTTPPKEAAKPTLMPRAQSYKDLTHLPVPTGKIFVSVYNIQDETGQFKPYPASNFSTAVPQSATAMLVTALKDSRW
FIPLERQGLQNLLNERKIIRAAQENGTVAINNRIPLPSLTAANIMVEGSIIGYESNVKSGGAGARYFGIGADTQYQL
DQIAVNLRVVNVSTGEILSSVNTSKTILSYEVQAGVFRFIDYQRLLEGEIGYTSNEPVMLCLMSAIETGVIFLINDG
IDRGLWDLQNKADRQNDILVKYRQMSVPPES
SEQ ID NO: 72
CLTAPPKEAAKPTLMPRAQSYRDLTHLPAPTGKIFVSVYNIQDETGQFKPYPASNFSTAVPQSATAMLVTALKDSHW
FIPLERQGLQNLLNERKIIRAAQENGTVANNNRMPLQSLAAANVMIEGSIIGYESNVKSGGVGARYFGIGADTQYQL
IDQAVNLRVVNVSTGEVLSSVNTSKTILSYEVQAGVFRFIDYQRLLEGEIGYTSNEPVMMCLMSAIETGVIELINDG
IDRGLWDLQNKADAQNPVLVKYRDMSVPPES
SEQ ID NO: 73
CLTAPPKEAAKPTLMPRAQSYRDLTHLPLPSGKVFVSVYNIQDETGQFKPYPASNFSTAVPQSATAMLVTALKDSRW
FVPLERQGLQNLLNERKIIRAAQENGTVADNNRIPLQSLTAANVMIEGSIIGYESNVKSGGVGARYFGIGADTQYQL
DQIAVNLRVVNVSTGEVLSSVNTSKTILSYEVQAGVFRFVDYQRLLEGEIGYTSNEPVMLCLMSAIETGVIYLINDG
IERGLWDLQQKADVDNPILARYRNMSAPPES
SEQ ID NO: 74
CLTAPPKEAAKPTLMPRAQSYRDLTNLPDPKGKLFVSVYNIQDETGQFKPYPASNFSTAVPQSATSMLVTALKDSRW
FPILERQGLQNLLNERKIIRAAQENGTVAENNRMPLQSLVAANVMIEGSIIGYESNVKSGGVGARYFGIGGDTQYQL
IDQAVNLRVVNVSTGEVLSSVNTSKTILSYEVQAGVFRFIDYQRLLEGEIGYTANEPVMLCLMSAIETGVIHLINDK
GINRGLWELNKGDAKNTILAKYRSMAVPPES
SEQ ID NO: 75
CLTAAPKEAARPTLLPRAPSYTDLTHLPSPQGRIFVSVYNIQDETGQFKPYPACNFSTAVPQSATAMLVSALKDSKW
FEIPLRQGLQNLLNERKIIRAAQENGSVAINNQRPLSSLVAANILIEGSIIGYESNVKSGGVGARYFGIGASTQYQL
IDQAVNLRAVDVNTGEVLSSVNTSKTILSYEVQAGVFRFIDYQRLLEGELGYTTNEPVMLCLMSAIESGVIYLVNDG
IERNLWQLQNPSEINSPILQRYKNNIVPAES
SEQ ID NO: 76
CITSPPKQAAKPTLLPRSQSYQDLTHLPEPQGRLFVSVYNISDETGQFKPYPASNFSTSVPQSATAMLVSALKDSNW
IFPLERQGLQNLLNERKIIRAAQENGTVAVNNRTQLPSLVAANILIEGSIIGYESNVKSGGAGARYFGIGASTQYQL
IDQAVNLRVVNVSTGEVLSSVNTSKTILSYEFQAGVFRYIDYQRLLEGEVGYTVNEPVMLCLMSAIETGVIYLVNDG
NISRLWQLKNASDINSPVLEKYKSIIVP
SEQ ID NO: 77
CLTAPPKQAAKPTLMPRAQSYQDLTHLPEPAGKLFVSVYNIQDETGQFKPYPASNFSTAVPQSATAMLVSALKDSGW
FIPLERQGLQNLLNERKIIRAAQENGTAAVNNQHQLSSLVAANVLVEGSIIGYESNVKSGGAGARFFGIGASTQYQL
IDQAVNLRVVDVNTGQVLSSVNTSKTILSYEVQAGVFRYIDYQRLLEGEIGYTTNEPVMLCVMSAIETGVIYLVNDG
INRNLWTLKNPQDAKSSVLERYKSTIVP
SEQ ID NO: 78
CITTPPQEAAKPTLLPRDATYKDLVSLPQPRGKIYVAVYNIQDETGQFQPYPASNFSTSVPQSATAMLVSSLKDSRW
FVPLERQGLNNLLNERKIIRAAQQNGTVGDNNASPLPSLYSANVIVEGSIIGYASNVKTGGFGARYFGIGGSTQYQL
DQVAVNLRIVNVHTGEVLSSVNTSKTILSYEIQAGVFRFIDYQRLLEGEAGFTTNEPVMTCLMSAIEEGVIHLINDG
INKKLWALSNAADINSEVLTRYRK
SEQ ID NO: 79
ITEVPKEAAKPTLMPRASTYKDLVALPKPNGKIIVSVYSVQDETGQFKPLPASNFSTAVPQSGNAMLTSALKDSGWF
VPLEREGLQNLLNERKIIRAAQENGTVAANNQQPLPSLLSANVVIEGAIIGYDSDIKTGGAGARYFGIGADGKYRVD
QVAVNLRAVDVRTGEVLLSVNTSKTILSSELSAGVFRFIEYQRLLELEAGYTTNEPVMMCMMSALEAGVAHLIVEGI
RQNLWSLQNPSDINNPIIQRYMKEDVP
SEQ ID NO: 80
PETSESPTLMQRGANYIDLISLPKPQGKIFVSVYDFRDQTGQYKPQPNSNFSTAVPQGGTALLTMALLDSEWFYPLE
RQGLQNLLTERKIIRAAQKKQESISNHGSTLPSLLSANVMIEGGIVAYDSNIKTGGAGARYLGIGGSGQYRADQVTV
NIRAVDVRSGKILTSVTTSKTILSYEVSAGAFRFVDYKELLEVELGYTNNEPVNIALMSAIDSAVIHLIVKGVQQGL
WRPANLDTRNNPIFKKY
SEQ ID NO: 81
PDASESPTLMQRGATYLDLISLPKPQGKIYVSVYDFRDQTGQYKPQPNSNFSTAVPQGGTALLTMALLDSEWFYPLE
RQGLQNLLTERKIIRAAQKKQESISNHGSTLPSLLSANVMIEGGIVAYDSNIKTGGAGARYLGIGGSGQYRADQVTV
NIRAVDVRSGKILTSVTTSKTILSYELSAGAFRFVDYKELLEVELGYTNNEPVNIALMSAIDSAVIHLIVKGIEEGL
WRPENQNGKENPIFRKY
SEQ ID NO: 82
PETSKEPTLMARGTAYQDLVSLPLPKGKVYVSVYDFRDQTGQYKPQPNSNFSTAVPQGGAALLTTALLDSRWFMPLE
REGLQNLLTERKIIRAAQKKDEIPTNHGVHLPSLASANIMVEGGIVAYDTNIQTGGAGARYLGVGASGQYRTDQVTV
NIRAVDVRTGRILLSVTTSKTILSKELQTGVFKFVDYKDLLEAELGYTTNEPVNLAVMSAIDAAVVHVIVDGIKTGL
WEPLRGEDLQHPIIQEYMNRSKP
SEQ ID NO: 83
CATHIGSPVADEKATLMPRSVSYKELISLPKPKGKIVAAVYDFRDQTGQYLPAPASNFSTAVTQGGVAMLSTALWDS
QWFVPLEREGLQNLLTERKIVRAAQNKPNVPGNNANQLPSLVAANILIEGGIVAYDSNVRTGGAGAKYFGIGASGEY
RVDQVTVNLRAVDIRSGRILNSVTTSKTVMSQQVQAGVFRFVEYKRLLEAEAGFSTNEPVQMCVMSAIESGVIRLIA
NGVRDNLWQLADQRDIDNPILQEYLQDNAP
SEQ ID NO: 84
ASSSLMPKGESYYDLINLPAPQGVMLAAVYDFRDQTGQYKPIPSSNFSTAVPQSGTAFLAQALNDSSWFIPVEREGL
QNLLTERKIVRAGLKGDANKLPQLNSAQILMEGGIVAYDTNVRTGGAGARYLGIGAATQFRVDTVTVNLRAVDIRTG
RLLSSVTTTKSILSKEITAGVFKFIDAQELLESELGYTSNEPVSLCVASAIESAVVHMIADGIWKGAWNLADQASGL
RSPVLQKY
SEQ ID NO: 85
QDSETPTLTPRASTYYDLINMPRPKGRLMAVVYGFRDQTGQYKPTPASSFSTSVTQGAASMLMDALSASGWFVVLER
EGLQNLLTERKIIRASQKKPDVAENIMGELPPLQAANLMLEGGIIAYDTNVRSGGEGARYLGIDISREYRVDQVTVN
LRAVDVRTGQVLANVMTSKTIYSVGRSAGVFKFIEFKKLLEAEVGYTTNEPAQLCVLSAIESAVGHLLAQGIEQRLW
QV
SEQ ID NO: 86
MPKSDTYYDLIGLPHPQGSMLAAVYDFRDQTGQYKAIPSSNFSTAVPQSGTAFLAQALNDSSWFVPVEREGLQNLLT
ERKIVRAGLKGEANQLPQLSSAQILMEGGIVAYDTNIKTGGAGARYLGIGVNSKFRVDTVTVNLRAVDIRTGRLLSS
VTTTKSILSKEVSAGVFKFIDAQDLLESELGYTSNEPVSLCVAQAIESAVVHMIADGIWKRAWNLADTASGLNNPVL
QKY
SEQ ID NO: 87
LTRRMSTYQDLIDMPAPRGKIVTAVYSFRDQSGQYKPAPSSSFSTAVTQGAAAMLVNVLNDSGWFIPLEREGLQNIL
TERKIIRAALKKDNVPVNNSAGLPSLLAANIMLEGGIVGYDSNIHTGGAGARYFGIGASEKYRVDEVTVNLRAIDIR
TGRILHSVLTSKKILSREIRSDVYRFIEFKHLLEMEAGITTNDPAQLCVLSAIESAVAHLIVDGVIKKSWSLADPNE
LNSPVIQAYQQQRI
SEQ ID NO: 88
PSDPERSTMGELTPSTAELRNLPLPNEKIVIGVYKFRDQTGQYKPSENGNNWSTAVPQGTTTILIKALEDSRWFIPI
ERENIANLLNERQIIRSTRQEYMKDADKNSQSLPPLLYAGILLEGGVISYDSNTMTGGFGARYFGIGASTQYRQDRI
TIYLRAVSTLNGEILKTVYTSKTILSTSVNGSFFRYIDTERLLEAEVGLTQNEPVQLAVTEAIEKAVRSLIIEGTRD
KIW
(DNA sequence encoding Pro-CP1-Eco-(WT-Y51A/F56Q/D149N/E185N/E201N/E203N-
StrepII(C)))
SEQ ID NO: 89
ATGCAGCGTCTGTTTCTGCTGGTCGCGGTGATGCTGCTGAGCGGTTGTCTGACCGCACCGCCGAAAGAAGCGGCA
CGTCCGACCCTGATGCCGCGTGCACAGAGCTATAAAGATCTGACCCATCTGCCGGCTCCGACGGGCAAAATCTTCG
TTTCTGTCTACAACATCCAGGACGAAACCGGTCAATTTAAACCAGCTCCTGCGTCAAATCAATCGACTGCCGTTCCG
CAGTCAGCAACCGCTATGCTGGTCACGGCACTGAAAGATTCGCGTTGGTTCATTCCGCTGGAACGCCAGGGCCTG
CAAAACCTGCTGAATGAACGTAAAATTATCCGCGCAGCTCAGGAAAACGGTACCGTGGCCATTAACAATCGCATC
CCGCTGCAAAGTCTGACGGCGGCCAACATCATGGTTGAAGGCTCCATTATCGGTTATGAAAGCAATGTCAAATCTG
GCGGTGTGGGCGCACGTTATTTCGGCATTGGTGCTAATACCCAGTACCAACTGGACCAGATCGCAGTTAACCTGC
GCGTGGTTAATGTCAGCACCGGCGAAATTCTGAGCTCTGTGAATACCAGTAAAACGATCCTGTCCTACAACGTGCA
GGCTGGTGTTTTTCGTTTCATTGATTATCAACGCCTGCTGAATGGCAACGTCGGTTACACCAGCAACGAACCGGTG
ATGCTGTGTCTGATGTCTGCGATTGAAACGGGTGTTATTTTTCTGATCAATGATGGCATCGACCGTGGTCTGTGGG
ATCTGCAGAACAAAGCGGAACGTCAAAATGACATTCTGGTGAAATACCGCCACATGTCAGTTCCGCCGGAAAGTT
CCGCATGGAGCCACCCGCAGTTCGAAAAA
(Amino acid sequence of Pro-CP1-Eco-(WT-Y51A/F56Q/D149N/E185N/E201N/E203N-
StrepII(C)))
SEQ ID NO: 90
MQRLFLLVAVMLLSGCLTAPPKEAARPTLMPRAQSYKDLTHLPAPTGKIFVSVYNIQDETGQFKPAPASNQSTAVPQ
SATAMLVTALKDSRWFIPLERQGLQNLLNERKIIRAAQENGTVAINNRIPLQSLTAANIMVEGSIIGYESNVKSGGV
GARYFGIGANTQYQLDQIAVNLRVVNVSTGEILSSVNTSKTILSYNVQAGVFRFIDYQRLLNGNVGYTSNEPVMLCL
MSAIETGVIFLINDGIDRGLWDLQNKAERUNDILVKYRHMSVPPESSAWSHPQFEK

### REFERENCES

Chin J W., Martin A B., King D S., Wang L., Schultz P G. (2002) Addition of a photocrosslinking amino acid to the genetic code of Escherichia coli. Proc Nat Acad Sci USA 99(17): 11020-11024.

Goyal P, Van Gerven N, Jonckheere W, Remaut H. (2013) Crystallization and preliminary X-ray crystallographic analysis of the curli transporter CsgG. Acta Crystallogr Sect F Struct Biol Cryst Commun. 69(Pt 12):1349-53.

Goyal P, Krasteva P V, Van Gerven N, Gubellini F, Van den Broeck I, Troupiotis-Tsaïlaki A, Jonckheere W, Péhau-Arnaudet G, Pinkner J S, Chapman M R, Hultgren S J, Howorka S, Fronzes R, Remaut H. (2014) Structural and mechanistic insights into the bacterial amyloid secretion channel CsgG. Nature 516(7530):250-3.

Hammar M, Arnqvist A, Bian Z, Olsén A, Normark S. (1995) Expression of two csg operons is required for production of fibronectin- and congo red-binding curli polymers in Escherichia coli K-12. Mol Microbiol. 18(4):661-70.

Juncker A S, Willenbrock H, Von Heijne G, Brunak S, Nielsen H, Krogh A. (2003) Prediction of lipoprotein signal peptides in Gram-negative bacteria. Protein Sci. 12(8):1652-62.

Ludtke S J. 2016, Single-particle refinement and variability analysis in EMAN2.1. Methods Enzymol. 579:159-89.

Rohou A and Grigorieff N 2015, CTFFIND4: Fast and accurate defocus estimation from electron micrographs. J Struct Biol. 192(2):216-21.

Robinson L S, Ashman E M, Hultgren S J, Chapman M R. (2006) Secretion of curli fibre subunits is mediated by the outer membrane-localized CsgG protein. Molecular Microbiology 59, 870-881.

Scheres 2012, RELION: implementation of a Bayesian approach to cryo-EM structure determination. J. Struct. Biol. 180(3):519-30.

Wang A., Winblade Nairn N., Marelli M., Grabstein K. (2012). Protein Engineering with Non-Natural Amino Acids. Protein Engineering, Prof. Pravin Kaumaya (Ed.), InTech, DOI: 10.5772/28719.

Zheng S Q., Palovcak E., Armache J-P., Verba K A., Cheng Y., Agard D A. (2017) MotionCor2: anisotropic correction of beam-induced

1. A system for characterising a target polynucleotide, the system comprising a membrane and a pore complex;
wherein the pore complex comprises: (i) a nanopore located in the membrane, and (ii) an auxiliary protein or peptide attached to the nanopore;
wherein the nanopore and the auxiliary protein or peptide together form a continuous channel across the membrane, the channel comprising a first constriction region and a second constriction region;
wherein the first constriction region is formed by a portion of the nanopore, and wherein the second constriction region is formed by at least a portion of the auxiliary protein or peptide.

2. The system according to claim 1, wherein the auxiliary protein or peptide:
(i) is a multimeric protein;
(ii) does not naturally form a nanopore in a membrane and/or does not comprise a component, or a fragment thereof, of a transmembrane pore complex that forms naturally in a membrane;
(iii) is ring-shaped;
(iv) is selected from GroES, CsgF, pentraxin, SP1, and functional homologues and fragments thereof;
(v) is a transmembrane protein nanopore or a fragment thereof; optionally wherein the transmembrane protein pore is selected from MspA, α-hemolysin, CsgG, lysenin, InvG, GspD, leukocidin, FraC, aerolysin, NetB, and functional homologues and fragments thereof; or
(vi) comprises a fragment of a component of a transmembrane protein pore complex (wherein, when the nanopore is a CsgG pore, the fragment is not a fragment of CsgF).

3.-8. (canceled)

9. The system according to claim 1, wherein at least a portion of the auxiliary protein or peptide is located within the lumen of the nanopore.

10. The system according to claim 1, wherein the second constriction is formed by at least a portion of the auxiliary protein or peptide, which portion is located within the lumen of the nanopore.

11. The system according to claim 1, wherein the auxiliary protein or peptide is located entirely within the lumen of the nanopore.

12. The system according to claim 1, wherein the auxiliary protein or peptide is located outside the lumen of the nanopore.

13. The system according to claim 1, wherein the auxiliary protein or peptide is attached to the nanopore via one or more covalent bonds.

14. The system according to claim 1, wherein the auxiliary protein or peptide is attached to the nanopore via one or more non-covalent interactions.

15. The system according to claim 1, wherein the auxiliary protein is a modified auxiliary protein or peptide comprising at least one amino acid modification compared to the corresponding naturally occurring auxiliary protein or peptide; optionally wherein the modified auxiliary protein or peptide comprises: (i) at least one amino acid residue at the interface between the transmembrane protein nanopore and the auxiliary protein or peptide, which amino acid residue is not present in the corresponding naturally occurring auxiliary protein or peptide; and/or (ii) at least one amino acid residue that forms part of the second constriction, which amino acid residue is not present in the corresponding naturally occurring auxiliary protein or peptide.

16. (canceled)

17. The system according claim 1, wherein the first constriction and/or the second constriction has a minimum diameter of from about 0.5 nm to about 2 nm.

18. The system according to claim 1, wherein the membrane comprises a layer of amphipathic molecules.

19. The system according to claim 1, wherein the membrane is a solid state layer; optionally wherein the nanopore is a solid state nanopore formed in the solid state layer.

20. The system according to claim 1, wherein the nanopore is a transmembrane protein nanopore; optionally wherein the transmembrane protein nanopore is selected from MspA, α-hemolysin, CsgG, lysenin, InvG, GspD, leukocidin, FraC, aerolysin, NetB, and functional homologues and fragments thereof.

21. (canceled)

22. The system according to claim 20, wherein the nanopore is a first transmembrane protein nanopore and the auxiliary protein is a second transmembrane protein nanopore, or a fragment thereof.

23. The system according to claim 22, wherein the first transmembrane protein nanopore, and the second transmembrane protein nanopore, or fragment thereof, are of the same transmembrane protein nanopore type; optionally wherein the first transmembrane protein nanopore and the second transmembrane protein nanopore, or fragment thereof, are the same.

24. The system according to claim 22, wherein the first transmembrane protein nanopore and/or the second transmembrane protein nanopore or fragment thereof, are homooligomers.

25. The system according to claim 22, wherein the first transmembrane protein nanopore and/or the second transmembrane protein nanopore, or fragment thereof, are heterooligomers.

26. (canceled)

27. The system according to claim 22, wherein the first transmembrane protein nanopore and the second transmembrane protein nanopore, or fragment thereof, are of different transmembrane protein nanopore types.

28. The system according to claim 20, wherein the nanopore is a transmembrane protein nanopore selected from MspA, CsgG, and functional homologues and fragments thereof, and wherein the auxiliary protein is GroES or a functional homologue or fragment thereof.

29. The system according to claim 20, wherein the nanopore is a modified transmembrane protein nanopore comprising at least one amino acid modification compared to the corresponding naturally occurring transmembrane protein nanopore; optionally wherein the modified transmembrane protein nanopore comprises: (i) at least one amino acid residue at the interface between the transmembrane protein nanopore and the auxiliary protein, which amino acid residue is not present in the corresponding naturally occurring transmembrane protein nanopore; and/or (ii) at least one amino acid residue that forms part of the first constriction, which amino acid residue is not present in the corresponding naturally occurring transmembrane protein nanopore.

30.-34. (canceled)

35. The system according to claim 1, further comprising: an electrically-conductive solution in contact with the nanopore, electrodes providing a voltage potential across the membrane, and a measurement system for measuring the current through the nanopore.

36. An isolated pore complex comprising (i) a nanopore, and (ii) an auxiliary protein or peptide attached to the nanopore;
wherein the nanopore and the auxiliary protein or peptide together define a continuous channel, the channel comprising a first constriction region and a second constriction region;
wherein the first constriction region is formed by a portion of the nanopore, and wherein the second constriction region is formed by at least a portion of the auxiliary protein or peptide.

37. A method for characterising a target polynucleotide, the method comprising the steps of:
(a) contacting a system according to claim 1 with the target polynucleotide;
(b) applying a potential across the membrane such that the target polynucleotide enters the continuous channel formed by the pore complex; and
(c) taking one or more measurements as the polynucleotide moves with respect to the continuous channel, thereby characterising the polynucleotide.

38. The method according to claim 37, wherein step (c) comprises measuring the current passing through the continuous channel , wherein the current is indicative of the presence and/or one or more characteristics of the target polynucleotide and thereby detecting and/or characterising the target polynucleotide; optionally wherein the nucleotides in the target polynucleotide interact with the first and second constriction regions within the continuous channel and wherein each of the first and second constriction regions is capable of discriminating between different nucleotides, such that the overall current passing through the continuous channel is influenced by the interactions between each of the first and second constriction regions and the nucleotides located at each of the regions.

39.-43. (canceled)


SEQUENCE LISTING (112 sequences)

SEQ ID NO: 1 - DNA, 834 bp, Artificial Sequence
  P0AEA2; coding sequence for WT CsgG from E. coli K12
atgcagcgcttatttcttttggttgccgtcatgttactgagcggatgcttaaccgccccgcctaaagaagccgccagaccgacattaatgcctcgtgctcagagctacaaagatttgacccatctgccagcgccgacgggtaaaatctttgtttcggtatacaacattcaggacgaaaccgggcaatttaaaccctacccggcaagtaacttctccactgctgttccgcaaagcgccacggcaatgctggtcacggcactgaaagattctcgctggtttataccgctggagcgccagggcttacaaaacctgcttaacgagcgcaagattattcgtgcggcacaagaaaacggcacggttgccattaataaccgaatcccgctgcaatctttaacggcggcaaatatcatggttgaaggttcgattatcggttatgaaagcaacgtcaaatctggcggggttggggcaagatattttggcatcggtgccgacacgcaataccagctcgatcagattgccgtgaacctgcgcgtcgtcaatgtgagtaccggcgagatcctttcttcggtgaacaccagtaagacgatactttcctatgaagttcaggccggggttttccgctttattgactaccagcgcttgcttgaaggggaagtgggttacacctcgaacgaacctgttatgctgtgcctgatgtcggctatcgaaacaggggtcattttcctgattaatgatggtatcgaccgtggtctgtgggatttgcaaaataaagcagaacggcagaatgacattctggtgaaataccgccatatgtcggttccaccggaatcctga

SEQ ID NO: 2 - protein, 277 aa, Artificial Sequence
  P0AEA2 (1277); WT prepro CsgG from E. coli K12
MQRLFLLVAVMLLSGCLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 3 - protein, 262 aa, Artificial Sequence
  P0AEA2 (16277); mature CsgG from E. coli K12
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 4 - DNA, 414 bp, Artificial Sequence
  P0AE98; coding sequence for WT CsgF from E. coli K12
atgcgtgtcaaacatgcagtagttctactcatgcttatttcgccattaagttgggctggaaccatgactttccagttccgtaatccaaactttggtggtaacccaaataatggcgcttttttattaaatagcgctcaggcccaaaactcttataaagatccgagctataacgatgactttggtattgaaacaccctcagcgttagataactttactcaggccatccagtcacaaattttaggtgggctactgtcgaatattaataccggtaaaccgggccgcatggtgaccaacgattatattgtcgatattgccaaccgcgatggtcaattgcagttgaacgtgacagatcgtaaaaccggacaaacctcgaccatccaggtttcgggtttacaaaataactcaaccgatttt

SEQ ID NO: 5 - protein, 138 aa, Artificial Sequence
  P0AE98 (1138); WT pre CsgF from E. coli K12
MRVKHAVVLLMLISPLSWAGTMTFQFRNPNFGGNPNNGAFLLNSAQAQNSYKDPSYNDDFGIETPSALDNFTQAIQSQILGGLLSNINTGKPGRMVTNDYIVDIANRDGQLQLNVTDRKTGQTSTIQVSGLQNNSTDF

SEQ ID NO: 6 - protein, 119 aa, Artificial Sequence
  P0AE98 (20138); WT mature CsgF from E. coli K12
GTMTFQFRNPNFGGNPNNGAFLLNSAQAQNSYKDPSYNDDFGIETPSALDNFTQAIQSQILGGLLSNINTGKPGRMVTNDYIVDIANRDGQLQLNVTDRKTGQTSTIQVSGLQNNSTDF

SEQ ID NO: 7 - DNA, 106 bp, Artificial Sequence
  P0AE98; coding sequence for CsgF 127_6His
atgcgtgtcaaacatgcagtagttctactcatgcttatttcgccattaagttgggctggaaccatgactttccagttccgtcatcaccatcaccatcactaagccc

SEQ ID NO: 8 - protein, 33 aa, Artificial Sequence
  P0AE98 (128); preprotein of CsgF 2027_6His
MRVKHAVVLLMLISPLSWAGTMTFQFRHHHHHH

SEQ ID NO: 9 - DNA, 139 bp, Artificial Sequence
  P0AE98; coding sequence for CsgF 138_6His
atgcgtgtcaaacatgcagtagttctactcatgcttatttcgccattaagttgggctggaaccatgactttccagttccgtaatccaaactttggtggtaacccaaataatggccatcaccatcaccatcactaagccc

SEQ ID NO: 10 - protein, 44 aa, Artificial Sequence
  P0AE98 (139); preprotein of CsgF 2038_6His
MRVKHAVVLLMLISPLSWAGTMTFQFRNPNFGGNPNNGHHHHHH

SEQ ID NO: 11 - DNA, 169 bp, Artificial Sequence
  P0AE98; coding sequence for CsgF 148_6His
atgcgtgtcaaacatgcagtagttctactcatgcttatttcgccattaagttgggctggaaccatgactttccagttccgtaatccaaactttggtggtaacccaaataatggcgcttttttattaaatagcgctcaggcccaacatcaccatcaccatcactaagccc

SEQ ID NO: 12 - protein, 54 aa, Artificial Sequence
  P0AE98 (149); preprotein of CsgF 2048_6His
MRVKHAVVLLMLISPLSWAGTMTFQFRNPNFGGNPNNGAFLLNSAQAQHHHHHH

SEQ ID NO: 13 - DNA, 217 bp, Artificial Sequence
  P0AE98; coding sequence for CsgF 164_6His
atgcgtgtcaaacatgcagtagttctactcatgcttatttcgccattaagttgggctggaaccatgactttccagttccgtaatccaaactttggtggtaacccaaataatggcgcttttttattaaatagcgctcaggcccaaaactcttataaagatccgagctataacgatgactttggtattgaaacacatcaccatcaccatcactaagccc

SEQ ID NO: 14 - protein, 70 aa, Artificial Sequence
  P0AE98 (165); preprotein of CsgF 2064_6His
MRVKHAVVLLMLISPLSWAGTMTFQFRNPNFGGNPNNGAFLLNSAQAQNSYKDPSYNDDFGIETHHHHHH

SEQ ID NO: 15 - protein, 34 aa, Artificial Sequence
  P0AE98 (2053); mature peptide of CsgF 2053
GTMTFQFRNPNFGGNPNNGAFLLNSAQAQNSYKD

SEQ ID NO: 16 - protein, 25 aa, Artificial Sequence
  P0AE98 (2042); mature peptide of CsgF 2042+KD
GTMTFQFRNPNFGGNPNNGAFLLKD

SEQ ID NO: 17 - protein, 32 aa, Artificial Sequence
  Q88H88_PSEPK (2355)
TELVYTPVNPAFGGNPLNGTWLLNNAQAQNDY

SEQ ID NO: 18 - protein, 32 aa, Artificial Sequence
  A0A143HJA0_9GAMM (2557)
TELIYEPVNPNFGGNPLNGSYLLNNAQAQDRH

SEQ ID NO: 19 - protein, 32 aa, Artificial Sequence
  Q5E245_VIBF1 (2153)
SELVYTPVNPNFGGNPLNTSHLFGGANAINDY

SEQ ID NO: 20 - protein, 32 aa, Artificial Sequence
  Q084E5_SHEFN (1951)
TQLVYTPVNPAFGGSYLNGSYLLANASAQNEH

SEQ ID NO: 21 - protein, 32 aa, Artificial Sequence
  F0LZU2_VIBFN (1547)
SSLVYEPVNPTFGGNPLNTTHLFSRAEAINDY

SEQ ID NO: 22 - protein, 32 aa, Artificial Sequence
  A0A136HQR0_9ALTE (2658)
TELVYEPINPSFGGNPLNGSFLLSKANSQNAH

SEQ ID NO: 23 - protein, 32 aa, Artificial Sequence
  A0A0W1SRL3_9GAMM (2153)
TEIVYQPINPSFGGNPMNGSFLLQKAQSQNAH

SEQ ID NO: 24 - protein, 33 aa, Artificial Sequence
  B0UH01_METS4 (2659)
SSLVYQPVNPAFGGPQLNGSWLQAEANAQNIPQ

SEQ ID NO: 25 - protein, 31 aa, Artificial Sequence
  Q6NAU5_RHOPA (2253)
GSLVYTPTNPAFGGSPLNGSWQMQQATAGNH

SEQ ID NO: 26 - protein, 32 aa, Artificial Sequence
  G8PUY5_PSEUV (738)
QQLIYQPTNPSFGGYAANTTHLFATANAQKTA

SEQ ID NO: 27 - protein, 32 aa, Artificial Sequence
  A0A0S2ETP7_9RHIZ (2557)
GDLVYTPVNPSFGGSPLNSAHLLSIAGAQKNA

SEQ ID NO: 28 - protein, 32 aa, Artificial Sequence
  E3I1Z1_RHOVT (1951)
AELGYTPVNPSFGGSPLNGSTLLSEASAQKPN

SEQ ID NO: 29 - protein, 31 aa, Artificial Sequence
  F3Z094_DESAF (2455)
TELVFSFTNPSFGGDPMIGNFLLNKADSQKR

SEQ ID NO: 30 - protein, 32 aa, Artificial Sequence
  A0A176T7M2_9FLAO (2153)
QQLVYKSINPFFGGGDSFAYQQLLASANAQND

SEQ ID NO: 31 - protein, 31 aa, Artificial Sequence
  D2QPP8_SPILD (1445)
QALVYHPNNPAFGGNTFNYQWMLSSAQAQDR

SEQ ID NO: 32 - protein, 32 aa, Artificial Sequence
  N2IYT1_9PSED (2658)
TELVYTPKNPAFGGSPLNGSYLLGNAQAQNDY

SEQ ID NO: 33 - protein, 32 aa, Artificial Sequence
  W7QHV5_9GAMM (2658)
GQLIYQPINPSFGGDPLLGNHLLNKAQAQDTK

SEQ ID NO: 34 - protein, 32 aa, Artificial Sequence
  D4ZLW2_SHEVD (2355)
TQLIYTPVNPNFGGSYLNGSYLLANASVQNDH

SEQ ID NO: 35 - protein, 32 aa, Artificial Sequence
  D2QT92_SPILD (2153)
QAFVYHPNNPNFGGNTFNYSWMLSSAQAQDRT

SEQ ID NO: 36 - protein, 31 aa, Artificial Sequence
  A0A167UJA2_9FLAO (2051)
QGLIYKPKNPAFGGDTFNYQWLASSAESQNK

SEQ ID NO: 37 - protein, 8 aa, Artificial Sequence
  P0AE98 (2028); mature peptide of CsgF 2027
GTMTFQFR

SEQ ID NO: 38 - protein, 19 aa, Artificial Sequence
  P0AE98 (2039); mature peptide of CsgF 2038
GTMTFQFRNPNFGGNPNNG

SEQ ID NO: 39 - protein, 29 aa, Artificial Sequence
  P0AE98 (2049); mature peptide of CsgF 2048
GTMTFQFRNPNFGGNPNNGAFLLNSAQAQ

SEQ ID NO: 40 - protein, 45 aa, Artificial Sequence
  P0AE98 (2065); mature peptide of CsgF 2064
GTMTFQFRNPNFGGNPNNGAFLLNSAQAQNSYKDPSYNDDFGIET

SEQ ID NO: 41 - DNA, 24 bp, Artificial Sequence
  Primer CsgF_d27_end
acggaactggaaagtcatggttcc

SEQ ID NO: 42 - DNA, 30 bp, Artificial Sequence
  Primer CsgF_d38_end
gccattatttgggttaccaccaaagtttgg

SEQ ID NO: 43 - DNA, 30 bp, Artificial Sequence
  Primer CsgF_d48_end
ttgggcctgagcgctatttaataaaaaagc

SEQ ID NO: 44 - DNA, 33 bp, Artificial Sequence
  Primer CsgF_d64_end
tgtttcaataccaaagtcatcgttatagctcgg

SEQ ID NO: 45 - DNA, 25 bp, Artificial Sequence
  Primer pNa62_CsgF_histag_Fw
catcaccatcaccatcactaagccc

SEQ ID NO: 46 - DNA, 32 bp, Artificial Sequence
  Primer CsgF-His_pET22b_FW
cccccatatgggaaccatgactttccagttcc

SEQ ID NO: 47 - DNA, 55 bp, Artificial Sequence
  Primer CsgF-His_pET22b_Rev
ccccgaattcctaatggtgatggtgatggtggtaaaaatcggttgagttattttg

SEQ ID NO: 48 - DNA, 58 bp, Artificial Sequence
  Primer csgEFG_pDONR221_FW
ggggacaagtttgtacaaaaaagcaggctacctcaggcgataaagccatgaaacgtta

SEQ ID NO: 49 - DNA, 72 bp, Artificial Sequence
  Primer csgEFG_pDONR221_Rev
ggggaccactttgtacaagaaagctgggtgtttaaactcatttttcgaactgcgggtggctccaagcgctgg

SEQ ID NO: 50 - DNA, 59 bp, Artificial Sequence
  Primer Mut_csgF_His_FW
caaaataactcaaccgattttcatcaccatcaccatcactaagccccagcttcataagg

SEQ ID NO: 51 - DNA, 59 bp, Artificial Sequence
  Primer Mut_csgF_His_Rev
ccttatgaagctggggcttagtgatggtgatggtgatgaaaatcggttgagttattttg

SEQ ID NO: 52 - DNA, 21 bp, Artificial Sequence
  Primer DelCsgE_Rev
agcctgcttttttgtacaaac

SEQ ID NO: 53 - DNA, 23 bp, Artificial Sequence
  Primer DelCsgE FW
ataaaaaattgttcggaggctgc

SEQ ID NO: 54 - protein, 30 aa, Artificial Sequence
  P0AE98 (2050); mature peptide of CsgF 130
GTMTFQFRNPNFGGNPNNGAFLLNSAQAQN

SEQ ID NO: 55 - protein, 35 aa, Artificial Sequence
  P0AE98 (2054); mature peptide of CsgF 135
GTMTFQFRNPNFGGNPNNGAFLLNSAQAQNSYKDP

SEQ ID NO: 56 - protein, 155 aa, Artificial Sequence
  Pro-CsgF-Eco-(WT-T4C/N17S/P35-TEV-S36)-StrepII
MRVKHAVVLLMLISPLSWAGTMCFQFRNPNFGGNPSNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 57 - protein, 155 aa, Artificial Sequence
  Pro-CsgF-Eco-(WT-N17S-Del (P35-[TEV]-S36)-StrepII
MRVKHAVVLLMLISPLSWAGTMTFQFRNPNFGGNPSNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 58 - protein, 155 aa, Artificial Sequence
  Pro-CsgF-Eco-(WT-G1C/N17S/P35-[TEV]-S36)- StrepII
MRVKHAVVLLMLISPLSWACTMTFQFRNPNFGGNPSNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 59 - protein, 155 aa, Artificial Sequence
  Pro-CsgF-Eco-(WT-G1C/P35-[TEV]-S36)-StrepII
MRVKHAVVLLMLISPLSWACTMTFQFRNPNFGGNPNNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 60 - protein, 155 aa, Artificial Sequence
  Pro-CsgF-Eco-(WT-T45-TEV-P46)-H10
MRVKHAVVLLMLISPLSWAGTMTFQFRNPNFGGNPNNGAFLLNSAQAQNSYKDPSYNDDFGIETENLYFQSPSALDNFTQAIQSQILGGLLSNINTGKPGRMVTNDYIVDIANRDGQLQLNVTDRKTGQTSTIQVSGLQNNSTDFHHHHHHHHHH

SEQ ID NO: 61 - protein, 155 aa, Artificial Sequence
  Pro-CsgF-Eco-(WT-P35-TEV-S36)-H10
MRVKHAVVLLMLISPLSWAGTMTFQFRNPNFGGNPNNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFHHHHHHHHHH

SEQ ID NO: 62 - protein, 155 aa, Artificial Sequence
  Pro-CsgF-Eco-(WT-N30-TEV-S31)-H10
MRVKHAVVLLMLISPLSWAGTMTFQFRNPNFGGNPNNGAFLLNSAQAQNENLYFQSSYKDPSYNDDFGIETPSALDNFTQAIQSQILGGLLSNINTGKPGRMVTNDYIVDIANRDGQLQLNVTDRKTGQTSTIQVSGLQNNSTDFHHHHHHHHHH

SEQ ID NO: 63 - protein, 149 aa, Artificial Sequence
  Pro-CsgF-Eco-(WT-T45-TEV-F51)-H10
MRVKHAVVLLMLISPLSWAGTMTFQFRNPNFGGNPNNGAFLLNSAQAQNSYKDPSYNDDFGIETENLYFQSFTQAIQSQILGGLLSNINTGKPGRMVTNDYIVDIANRDGQLQLNVTDRKTGQTSTIQVSGLQNNSTDFHHHHHHHHHH

SEQ ID NO: 64 - protein, 149 aa, Artificial Sequence
  Pro-CsgF-Eco-(WT-N30-TEV-Y37)-H10
MRVKHAVVLLMLISPLSWAGTMTFQFRNPNFGGNPNNGAFLLNSAQAQNENLYFQSYNDDFGIETPSALDNFTQAIQSQILGGLLSNINTGKPGRMVTNDYIVDIANRDGQLQLNVTDRKTGQTSTIQVSGLQNNSTDFHHHHHHHHHH

SEQ ID NO: 65 - protein, 155 aa, Artificial Sequence
  Pro-CsgF-Eco-(WT-D34-[C3]-S36)
MRVKHAVVLLMLISPLSWACTMTFQFRNPNFGGNPNNGAFLLNSAQAQNSYKDLEVLFQGPSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 66 - protein, 156 aa, Artificial Sequence
  Pro-CsgF-Eco-(WT-I42-[C3]-E43)
MRVKHAVVLLMLISPLSWACTMTFQFRNPNFGGNPNNGAFLLNSAQAQNSYKDPSYNDDFGILEVLFQGPETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 67 - protein, 148 aa, Artificial Sequence
  Pro-CsgF-Eco-(WT-N38-[C3]-S47)
MRVKHAVVLLMLISPLSWACTMTFQFRNPNFGGNPNNGAFLLNSAQAQNSYKDPSYNLEVLFQGPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 68 - protein, 248 aa, Artificial Sequence
  YP_001453594.1 1-248 of hypothetical protein CKO_02032 [Citrobacter koseri ATCC BAA-895]
MPRAQSYKDLTHLPMPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKAERQNDILVKYRHMSVPPES

SEQ ID NO: 69 - protein, 223 aa, Artificial Sequence
  WP_001787128.1 16-238 of curli production assembly/transport component CsgG, partial [Salmonella enterica]
CLTAPPKQAAKPTLMPRAQSYKDLTHLPAPTGKIFVSVYNIQDETGQFKPYPASNFSTAVPQSATAMLVTALKDSRWFIPLERQGLQNLLNERKIIRAAQENGTVAMNNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETG

SEQ ID NO: 70 - protein, 262 aa, Artificial Sequence
  KEY44978.1 16-277 of curli production assembly/transport protein CsgG [Citrobacter amalonaticus]
CLTAPPKEAAKPTLMPRAQSYKDLTHLPIPTGKIFVSVYNIQDETGQFKPYPASNFSTAVPQSATAMLVTALKDSRWFVPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKADRQNDILVKYRHMSVPPES

SEQ ID NO: 71 - protein, 262 aa, Artificial Sequence
  YP_003364699.1 16-277 of curli production assembly/transport component [Citrobacter rodentium ICC168]
CLTTPPKEAAKPTLMPRAQSYKDLTHLPVPTGKIFVSVYNIQDETGQFKPYPASNFSTAVPQSATAMLVTALKDSRWFIPLERQGLQNLLNERKIIRAAQENGTVAINNRIPLPSLTAANIMVEGSIIGYESNVKSGGAGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKADRQNDILVKYRQMSVPPES

SEQ ID NO: 72 - protein, 262 aa, Artificial Sequence
  YP_004828099.1 16-277 of curli production assembly/transport component CsgG [Enterobacter asburiae LF7a]
CLTAPPKEAAKPTLMPRAQSYRDLTHLPAPTGKIFVSVYNIQDETGQFKPYPASNFSTAVPQSATAMLVTALKDSHWFIPLERQGLQNLLNERKIIRAAQENGTVANNNRMPLQSLAAANVMIEGSIIGYESNVKSGGVGARYFGIGADTQYQLDQIAVNLRVVNVSTGEVLSSVNTSKTILSYEVQAGVFRFIDYQRLLEGEIGYTSNEPVMMCLMSAIETGVIFLINDGIDRGLWDLQNKADAQNPVLVKYRDMSVPPES

SEQ ID NO: 73 - protein, 262 aa, Artificial Sequence
  WP_006819418.1 19-280 of transporter [Yokenella regensburgei]
CLTAPPKEAAKPTLMPRAQSYRDLTHLPLPSGKVFVSVYNIQDETGQFKPYPASNFSTAVPQSATAMLVTALKDSRWFVPLERQGLQNLLNERKIIRAAQENGTVADNNRIPLQSLTAANVMIEGSIIGYESNVKSGGVGARYFGIGADTQYQLDQIAVNLRVVNVSTGEVLSSVNTSKTILSYEVQAGVFRFVDYQRLLEGEIGYTSNEPVMLCLMSAIETGVIYLINDGIERGLWDLQQKADVDNPILARYRNMSAPPES

SEQ ID NO: 74 - protein, 262 aa, Artificial Sequence
  WP_024556654.1 16-277 of curli production assembly/transport protein CsgG [Cronobacter pulveris]
CLTAPPKEAAKPTLMPRAQSYRDLTNLPDPKGKLFVSVYNIQDETGQFKPYPASNFSTAVPQSATSMLVTALKDSRWFIPLERQGLQNLLNERKIIRAAQENGTVAENNRMPLQSLVAANVMIEGSIIGYESNVKSGGVGARYFGIGGDTQYQLDQIAVNLRVVNVSTGEVLSSVNTSKTILSYEVQAGVFRFIDYQRLLEGEIGYTANEPVMLCLMSAIETGVIHLINDGINRGLWELKNKGDAKNTILAKYRSMAVPPES

SEQ ID NO: 75 - protein, 262 aa, Artificial Sequence
  YP_005400916.1 16-277 of curli production assembly/transport protein CsgG [Rahnella aquatilis HX2]
CLTAAPKEAARPTLLPRAPSYTDLTHLPSPQGRIFVSVYNIQDETGQFKPYPACNFSTAVPQSATAMLVSALKDSKWFIPLERQGLQNLLNERKIIRAAQENGSVAINNQRPLSSLVAANILIEGSIIGYESNVKSGGVGARYFGIGASTQYQLDQIAVNLRAVDVNTGEVLSSVNTSKTILSYEVQAGVFRFIDYQRLLEGELGYTTNEPVMLCLMSAIESGVIYLVNDGIERNLWQLQNPSEINSPILQRYKNNIVPAES

SEQ ID NO: 76 - protein, 259 aa, Artificial Sequence
  KFC99297.1 20-278 of CsgG family curli production assembly/transport component [Kluyvera ascorbata ATCC 33433]
CITSPPKQAAKPTLLPRSQSYQDLTHLPEPQGRLFVSVYNISDETGQFKPYPASNFSTSVPQSATAMLVSALKDSNWFIPLERQGLQNLLNERKIIRAAQENGTVAVNNRTQLPSLVAANILIEGSIIGYESNVKSGGAGARYFGIGASTQYQLDQIAVNLRVVNVSTGEVLSSVNTSKTILSYEFQAGVFRYIDYQRLLEGEVGYTVNEPVMLCLMSAIETGVIYLVNDGISRNLWQLKNASDINSPVLEKYKSIIVP

SEQ ID NO: 77 - protein, 259 aa, Artificial Sequence
  KFC86716.116-274 of CsgG family curli production assembly/transport component [Hafnia alvei ATCC 13337]
CLTAPPKQAAKPTLMPRAQSYQDLTHLPEPAGKLFVSVYNIQDETGQFKPYPASNFSTAVPQSATAMLVSALKDSGWFIPLERQGLQNLLNERKIIRAAQENGTAAVNNQHQLSSLVAANVLVEGSIIGYESNVKSGGAGARFFGIGASTQYQLDQIAVNLRVVDVNTGQVLSSVNTSKTILSYEVQAGVFRYIDYQRLLEGEIGYTTNEPVMLCVMSAIETGVIYLVNDGINRNLWTLKNPQDAKSSVLERYKSTIVP

SEQ ID NO: 78 - protein, 255 aa, Artificial Sequence
  YP_007340845.116-270 of uncharacterised protein involved in formation of curli polymers [Enterobacteriaceae bacterium strain FGI 57]
CITTPPQEAAKPTLLPRDATYKDLVSLPQPRGKIYVAVYNIQDETGQFQPYPASNFSTSVPQSATAMLVSSLKDSRWFVPLERQGLNNLLNERKIIRAAQQNGTVGDNNASPLPSLYSANVIVEGSIIGYASNVKTGGFGARYFGIGGSTQYQLDQVAVNLRIVNVHTGEVLSSVNTSKTILSYEIQAGVFRFIDYQRLLEGEAGFTTNEPVMTCLMSAIEEGVIHLINDGINKKLWALSNAADINSEVLTRYRK

SEQ ID NO: 79 - protein, 258 aa, Artificial Sequence
  WP_010861740.1 17-274 of curli production assembly/transport protein CsgG [Plesiomonas shigelloides]
ITEVPKEAAKPTLMPRASTYKDLVALPKPNGKIIVSVYSVQDETGQFKPLPASNFSTAVPQSGNAMLTSALKDSGWFVPLEREGLQNLLNERKIIRAAQENGTVAANNQQPLPSLLSANVVIEGAIIGYDSDIKTGGAGARYFGIGADGKYRVDQVAVNLRAVDVRTGEVLLSVNTSKTILSSELSAGVFRFIEYQRLLELEAGYTTNEPVMMCMMSALEAGVAHLIVEGIRQNLWSLQNPSDINNPIIQRYMKEDVP

SEQ ID NO: 80 - protein, 248 aa, Artificial Sequence
  YP_205788.1  23-270 of curli production assembly/transport outer membrane lipoprotein component CsgG [Vibrio fischeri ES114]
PETSESPTLMQRGANYIDLISLPKPQGKIFVSVYDFRDQTGQYKPQPNSNFSTAVPQGGTALLTMALLDSEWFYPLERQGLQNLLTERKIIRAAQKKQESISNHGSTLPSLLSANVMIEGGIVAYDSNIKTGGAGARYLGIGGSGQYRADQVTVNIRAVDVRSGKILTSVTTSKTILSYEVSAGAFRFVDYKELLEVELGYTNNEPVNIALMSAIDSAVIHLIVKGVQQGLWRPANLDTRNNPIFKKY

SEQ ID NO: 81 - protein, 248 aa, Artificial Sequence
  WP_017023479.1 23-270 of curli production assembly protein CsgG [Aliivibrio logei]
PDASESPTLMQRGATYLDLISLPKPQGKIYVSVYDFRDQTGQYKPQPNSNFSTAVPQGGTALLTMALLDSEWFYPLERQGLQNLLTERKIIRAAQKKQESISNHGSTLPSLLSANVMIEGGIVAYDSNIKTGGAGARYLGIGGSGQYRADQVTVNIRAVDVRSGKILTSVTTSKTILSYELSAGAFRFVDYKELLEVELGYTNNEPVNIALMSAIDSAVIHLIVKGIEEGLWRPENQNGKENPIFRKY

SEQ ID NO: 82 - protein, 254 aa, Artificial Sequence
  WP_007470398.1 22-275 of Curli production assembly/transport component CsgG [Photobacterium sp. AK15]
PETSKEPTLMARGTAYQDLVSLPLPKGKVYVSVYDFRDQTGQYKPQPNSNFSTAVPQGGAALLTTALLDSRWFMPLEREGLQNLLTERKIIRAAQKKDEIPTNHGVHLPSLASANIMVEGGIVAYDTNIQTGGAGARYLGVGASGQYRTDQVTVNIRAVDVRTGRILLSVTTSKTILSKELQTGVFKFVDYKDLLEAELGYTTNEPVNLAVMSAIDAAVVHVIVDGIKTGLWEPLRGEDLQHPIIQEYMNRSKP

SEQ ID NO: 83 - protein, 261 aa, Artificial Sequence
  WP_021231638.1 17-277 of curli production assembly protein CsgG [Aeromonas veronii]
CATHIGSPVADEKATLMPRSVSYKELISLPKPKGKIVAAVYDFRDQTGQYLPAPASNFSTAVTQGGVAMLSTALWDSQWFVPLEREGLQNLLTERKIVRAAQNKPNVPGNNANQLPSLVAANILIEGGIVAYDSNVRTGGAGAKYFGIGASGEYRVDQVTVNLRAVDIRSGRILNSVTTSKTVMSQQVQAGVFRFVEYKRLLEAEAGFSTNEPVQMCVMSAIESGVIRLIANGVRDNLWQLADQRDIDNPILQEYLQDNAP

SEQ ID NO: 84 - protein, 239 aa, Artificial Sequence
  WP_033538267.1 27-265 of curli production assembly/transport protein CsgG [Shewanella sp. ECSMB14101]
ASSSLMPKGESYYDLINLPAPQGVMLAAVYDFRDQTGQYKPIPSSNFSTAVPQSGTAFLAQALNDSSWFIPVEREGLQNLLTERKIVRAGLKGDANKLPQLNSAQILMEGGIVAYDTNVRTGGAGARYLGIGAATQFRVDTVTVNLRAVDIRTGRLLSSVTTTKSILSKEITAGVFKFIDAQELLESELGYTSNEPVSLCVASAIESAVVHMIADGIWKGAWNLADQASGLRSPVLQKY

SEQ ID NO: 85 - protein, 233 aa, Artificial Sequence
  WP_003247972.1 30-262 of curli production assembly protein CsgG [Pseudomonas putida]
QDSETPTLTPRASTYYDLINMPRPKGRLMAVVYGFRDQTGQYKPTPASSFSTSVTQGAASMLMDALSASGWFVVLEREGLQNLLTERKIIRASQKKPDVAENIMGELPPLQAANLMLEGGIIAYDTNVRSGGEGARYLGIDISREYRVDQVTVNLRAVDVRTGQVLANVMTSKTIYSVGRSAGVFKFIEFKKLLEAEVGYTTNEPAQLCVLSAIESAVGHLLAQGIEQRLWQV

SEQ ID NO: 86 - protein, 234 aa, Artificial Sequence
  YP_003557438.1 1-234 of curli production assembly/transport component CsgG [Shewanella violacea DSS12]
MPKSDTYYDLIGLPHPQGSMLAAVYDFRDQTGQYKAIPSSNFSTAVPQSGTAFLAQALNDSSWFVPVEREGLQNLLTERKIVRAGLKGEANQLPQLSSAQILMEGGIVAYDTNIKTGGAGARYLGIGVNSKFRVDTVTVNLRAVDIRTGRLLSSVTTTKSILSKEVSAGVFKFIDAQDLLESELGYTSNEPVSLCVAQAIESAVVHMIADGIWKRAWNLADTASGLNNPVLQKY

SEQ ID NO: 87 - protein, 245 aa, Artificial Sequence
  WP_027859066.1 36-280 of curli production assembly/transport protein CsgG [Marinobacterium jannaschii]
LTRRMSTYQDLIDMPAPRGKIVTAVYSFRDQSGQYKPAPSSSFSTAVTQGAAAMLVNVLNDSGWFIPLEREGLQNILTERKIIRAALKKDNVPVNNSAGLPSLLAANIMLEGGIVGYDSNIHTGGAGARYFGIGASEKYRVDEVTVNLRAIDIRTGRILHSVLTSKKILSREIRSDVYRFIEFKHLLEMEAGITTNDPAQLCVLSAIESAVAHLIVDGVIKKSWSLADPNELNSPVIQAYQQQRI

SEQ ID NO: 88 - protein, 234 aa, Artificial Sequence
  CEJ70222.1 29-262 of Curli production assembly/transport component CsgG [Chryseobacterium oranimense G311]
PSDPERSTMGELTPSTAELRNLPLPNEKIVIGVYKFRDQTGQYKPSENGNNWSTAVPQGTTTILIKALEDSRWFIPIERENIANLLNERQIIRSTRQEYMKDADKNSQSLPPLLYAGILLEGGVISYDSNTMTGGFGARYFGIGASTQYRQDRITIYLRAVSTLNGEILKTVYTSKTILSTSVNGSFFRYIDTERLLEAEVGLTQNEPVQLAVTEAIEKAVRSLIIEGTRDKIW

SEQ ID NO: 89 - DNA, 861 bp, Artificial Sequence
  Pro-CP1-Eco-(WT-Y51A/F56Q/D149N/E185N/E201N/ E203N-StrepII( C))
atgcagcgtctgtttctgctggtcgcggtgatgctgctgagcggttgtctgaccgcaccgccgaaagaagcggcacgtccgaccctgatgccgcgtgcacagagctataaagatctgacccatctgccggctccgacgggcaaaatcttcgtttctgtctacaacatccaggacgaaaccggtcaatttaaaccagctcctgcgtcaaatcaatcgactgccgttccgcagtcagcaaccgctatgctggtcacggcactgaaagattcgcgttggttcattccgctggaacgccagggcctgcaaaacctgctgaatgaacgtaaaattatccgcgcagctcaggaaaacggtaccgtggccattaacaatcgcatcccgctgcaaagtctgacggcggccaacatcatggttgaaggctccattatcggttatgaaagcaatgtcaaatctggcggtgtgggcgcacgttatttcggcattggtgctaatacccagtaccaactggaccagatcgcagttaacctgcgcgtggttaatgtcagcaccggcgaaattctgagctctgtgaataccagtaaaacgatcctgtcctacaacgtgcaggctggtgtttttcgtttcattgattatcaacgcctgctgaatggcaacgtcggttacaccagcaacgaaccggtgatgctgtgtctgatgtctgcgattgaaacgggtgttatttttctgatcaatgatggcatcgaccgtggtctgtgggatctgcagaacaaagcggaacgtcaaaatgacattctggtgaaataccgccacatgtcagttccgccggaaagttccgcatggagccacccgcagttcgaaaaa

SEQ ID NO: 90 - protein, 287 aa, Artificial Sequence
  Pro-CP1-Eco-(WT-Y51A/F56Q/D149N/E185N/E201N/ E203N-StrepII( C))
MQRLFLLVAVMLLSGCLTAPPKEAARPTLMPRAQSYKDLTHLPAPTGKIFVSVYNIQDETGQFKPAPASNQSTAVPQSATAMLVTALKDSRWFIPLERQGLQNLLNERKIIRAAQENGTVAINNRIPLQSLTAANIMVEGSIIGYESNVKSGGVGARYFGIGANTQYQLDQIAVNLRVVNVSTGEILSSVNTSKTILSYNVQAGVFRFIDYQRLLNGNVGYTSNEPVMLCLMSAIETGVIFLINDGIDRGLWDLQNKAERQNDILVKYRHMSVPPESSAWSHPQFEK

SEQ ID NO: 91 - DNA, 45 bp, Artificial Sequence
  polyA DNA strand (SS20)
  misc_feature (45)..(45): 3′ biotinylation
aaaaaaaaaaaaaaaaaaaaaaaaaaaaaaaaaaaaaaaaaaaaa

SEQ ID NO: 92 - DNA, 45 bp, Artificial Sequence
  polyA DNA strand (SS21)
  misc_feature (44)..(44): Int C3 Spacer
  misc_feature (45)..(45): 3′ biotinylation
aaaaaaaaaaaaaaaaaaaaaaaaaaaaaaaaaaaaaaaaaaana

SEQ ID NO: 93 - DNA, 45 bp, Artificial Sequence
  polyA DNA strand (SS22)
  misc_feature (43)..(43): Int C3 Spacer
  misc_feature (45)..(45): 3′ biotinylation
aaaaaaaaaaaaaaaaaaaaaaaaaaaaaaaaaaaaaaaaaanaa

SEQ ID NO: 94 - DNA, 45 bp, Artificial Sequence
  polyA DNA strand (SS23)
  misc_feature (42)..(42): Int C3 Spacer
  misc_feature (45)..(45): 3′ biotinylation
aaaaaaaaaaaaaaaaaaaaaaaaaaaaaaaaaaaaaaaaanaaa

SEQ ID NO: 95 - DNA, 45 bp, Artificial Sequence
  polyA DNA strand (SS24)
  misc_feature (41)..(41): Int C3 Spacer
  misc_feature (45)..(45): 3′ biotinylation
aaaaaaaaaaaaaaaaaaaaaaaaaaaaaaaaaaaaaaaanaaaa

SEQ ID NO: 96 - DNA, 45 bp, Artificial Sequence
  polyA DNA strand (SS25)
  misc_feature (40)..(40): Int C3 Spacer
  misc_feature (45)..(45): 3′ biotinylation
aaaaaaaaaaaaaaaaaaaaaaaaaaaaaaaaaaaaaaanaaaaa

SEQ ID NO: 97 - DNA, 45 bp, Artificial Sequence
  polyA DNA strand (SS26)
  misc_feature (39)..(39): Int C3 Spacer
  misc_feature (45)..(45): 3′ biotinylation
aaaaaaaaaaaaaaaaaaaaaaaaaaaaaaaaaaaaaanaaaaaa

SEQ ID NO: 98 - DNA, 45 bp, Artificial Sequence
  polyA DNA strand (SS27)
  misc_feature (38)..(38): Int C3 Spacer
  misc_feature (45)..(45): 3′ biotinylation
aaaaaaaaaaaaaaaaaaaaaaaaaaaaaaaaaaaaanaaaaaaa

SEQ ID NO: 99 - DNA, 45 bp, Artificial Sequence
  polyA DNA strand (SS28)
  misc_feature (37)..(37): Int C3 Spacer
  misc_feature (45)..(45): 3′ biotinylation
aaaaaaaaaaaaaaaaaaaaaaaaaaaaaaaaaaaanaaaaaaaa

SEQ ID NO: 100 - DNA, 45 bp, Artificial Sequence
  polyA DNA strand (SS29)
  misc_feature (36)..(36): Int C3 Spacer
  misc_feature (45)..(45): 3′ biotinylation
aaaaaaaaaaaaaaaaaaaaaaaaaaaaaaaaaaanaaaaaaaaa

SEQ ID NO: 101 - DNA, 45 bp, Artificial Sequence
  polyA DNA strand (SS30)
  misc_feature (35)..(35): Int C3 Spacer
  misc_feature (45)..(45): 3′ biotinylation
aaaaaaaaaaaaaaaaaaaaaaaaaaaaaaaaaanaaaaaaaaaa

SEQ ID NO: 102 - DNA, 45 bp, Artificial Sequence
  polyA DNA strand (SS31)
  misc_feature (34)..(34): Int C3 Spacer
  misc_feature (45)..(45): 3′ biotinylation
aaaaaaaaaaaaaaaaaaaaaaaaaaaaaaaaanaaaaaaaaaaa

SEQ ID NO: 103 - DNA, 45 bp, Artificial Sequence
  polyA DNA strand (SS32)
  misc_feature (33)..(33): Int C3 Spacer
  misc_feature (45)..(45): 3′ biotinylation
aaaaaaaaaaaaaaaaaaaaaaaaaaaaaaaanaaaaaaaaaaaa

SEQ ID NO: 104 - DNA, 45 bp, Artificial Sequence
  polyA DNA strand (SS33)
  misc_feature (32)..(32): Int C3 Spacer
  misc_feature (45)..(45): 3′ biotinylation
aaaaaaaaaaaaaaaaaaaaaaaaaaaaaaanaaaaaaaaaaaaa

SEQ ID NO: 105 - DNA, 45 bp, Artificial Sequence
  polyA DNA strand (SS34)
  misc_feature (31)..(31): Int C3 Spacer
  misc_feature (45)..(45): 3′ biotinylation
aaaaaaaaaaaaaaaaaaaaaaaaaaaaaanaaaaaaaaaaaaaa

SEQ ID NO: 106 - DNA, 45 bp, Artificial Sequence
  polyA DNA strand (SS35)
  misc_feature (30)..(30): Int C3 Spacer
  misc_feature (45)..(45): 3′ biotinylation
aaaaaaaaaaaaaaaaaaaaaaaaaaaaanaaaaaaaaaaaaaaa

SEQ ID NO: 107 - DNA, 45 bp, Artificial Sequence
  polyA DNA strand (SS36)
  misc_feature (29)..(29): Int C3 Spacer
  misc_feature (45)..(45): 3′ biotinylation
aaaaaaaaaaaaaaaaaaaaaaaaaaaanaaaaaaaaaaaaaaaa

SEQ ID NO: 108 - DNA, 45 bp, Artificial Sequence
  polyA DNA strand (SS37)
  misc_feature (28)..(28): Int C3 Spacer
  misc_feature (45)..(45): 3′ biotinylation
aaaaaaaaaaaaaaaaaaaaaaaaaaanaaaaaaaaaaaaaaaaa

SEQ ID NO: 109 - DNA, 45 bp, Artificial Sequence
  polyA DNA strand (SS38)
  misc_feature (27)..(27): Int C3 Spacer
  misc_feature (45)..(45): 3′ biotinylation
aaaaaaaaaaaaaaaaaaaaaaaaaanaaaaaaaaaaaaaaaaaa

SEQ ID NO: 110 - protein, 20 aa, Artificial Sequence
  S-S complex fragment (sequence 1) identified by mass spectrometry (Fig 16B)
  DISULFID (11)..(11): Disulphide bonded to N terminal cysteine in CTMTFQFR
YFGIGADTQYCLDQIAVNLR

SEQ ID NO: 111 - protein, 8 aa, Artificial Sequence
  S-S complex fragment (sequence 2) identified by mass spectrometry (Fig 16B)
  DISULFID (1)..(1): Disulphide bonded to cysteine (residue 11) in YFGIGADTQYCLDQIAVNLR
CTMTFQFR

SEQ ID NO: 112 - DNA, 28 bp, Artificial Sequence
  Fragment of E. coli DNA containing T homopolymer for comparison of errors in deletions (Fig 26B)
cagtcgcatcggtttttactgcgggctg